(19) United States
(12) Patent Application Publication
(10) Pub. No.: US 2003/0228583 A1
(43) Pub. Date: Dec. 11, 2003

(54) Biomarkers of liver response

(76) Inventors: David E. Amacher, Lyme, CT (US); Lisa M. Fasulo, Ivoryton, CT (US); Herath Mudiyanselage Athula Chandrasiri Herath, Abingdon (GB); Gordon Duane Holt, Gaithersburg, MD (US); Thomas R. Stiger, Pawcatuck, CT (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/285,394

(22) Filed: Oct. 31, 2002

Related U.S. Application Data
(60) Provisional application No. 60/335,964, filed on Oct. 31, 2001.

(51) Int. Cl. C12Q 1/68; G01N 33/53; G01N 33/567; A61K 49/00

(52) U.S. Cl. 435/6; 435/7.2; 424/9.2

(57) ABSTRACT

The present invention provides methods and compositions for screening, diagnosing and prognosing liver response, for monitoring the effectiveness of liver response treatment, and for drug development.

### INTRODUCTION

The present invention is based on the identification of biomarkers that are associated with liver function and response to exogenous agents, including drugs and disease. In general, the invention features the use of identified genes and encoded proteins and binding partners thereto for drug screening and drug development and as diagnostics or prognostics of liver diseases or disorders

### BACKGROUND OF THE INVENTION

The liver is metabolically the most complex organ in the body and serves numerous vital functions. For example, the liver metabolizes ingested proteins, carbohydrates and fats for redistribution throughout the body. The liver also is the site of synthesis for key immune, inflammation and blood coagulation proteins, as well as locus of the processing of endotoxic and exotoxic molecules (e.g., hormones, drugs, viruses, chemicals, etc), which are commonly referred to as “xenobiotic” molecules. The liver's functions are in a state of continual flux as the organ responds to xenobiotics by rapidly mobilizing or curtailing metabolic pathways as required. Disruptions in the liver's responsiveness to environmental changes could lead to serious, and possibly life-threatening, consequences.

A wide variety of xenobiotics and biological agents can be disruptive to the liver, for example: chemical poisoning, such as that caused by alcohol; chemotherapeutics (e.g., a wide variety of approved treatments for cancer, infection, and inflammation, and with many experimental treatments undergoing clinical trial); environmental agents (e.g., vinyl chloride, paraquat, copper and iron). Viral infection can also disrupt liver function and lead to disease. Examples of virally induced liver disease include disease caused by hepatitis C virus, hepatitis B virus and hepatitis Delta virus. Autoimmunity can also disrupt liver function and lead to diseases, such as biliary atresia (such as is caused by anti-liver antigen antibodies) and rheumatoid arthritis. Genetic disease can cause liver dysfunction such as that caused by cystic fibrosis, alpha-1 antitrypsin deficiency or Wilson's and Menkes disease (such as related to defects in copper metabolism). Parasitic infection such as schistosomiasis, can produce liver dysfunction and disease. Liver dysfunction can also result from radiation-induced response during cancer chemotherapy.

The liver is an architecturally complex organ composed of more than a dozen unique cell types. Liver-disrupting xenobiotics may impact a single cell type exclusively, or, more commonly, may simultaneously interfere with multiple compartments. Thus, affected areas may range from highly focal to organ-wide lesions, and may spread or refocus over time. The intracellular response to xenobiotics may also change over time, for example beginning with the formation of lipid inclusions and transitioning to a collagen fiber deposition. The following is a list of alterations in liver function, many of them deleterious, that are considered herein as liver responses to xenobiotics and biological agents:

Detoxification pathway modulation—Hepatocyte response to xenobiotics such as drugs, chemicals, and other small molecules by the increased synthesis of detoxification pathway proteins such as cytochrome p450 and/or beta-glucuronidation enzymes. Xenobiotics modified by these pathway reactions may be rendered more toxic, for example by being converted to DNA- or protein-modifying species, and such toxicity may in turn, elicit hepatocyte response that e.g. further induces protein synthesis and/or DNA repair reactions, that may introduce unwanted changes to body structure or chemistry.

Steatosis—Vacuolar fat storage in hepatocytes that can lead to cell death. Since hepatocytes are distributed throughout the liver, steatosis is an organ-wide form of liver response.

Hepatocellular necrosis—Widespread, often rapid, hepatocyte cell death that generally is induced by hepatotoxic chemical agents such as acetaminophen.

Bile duct response/cholestasis—Focal response of the bile duct typically caused by blockage-inducing agents such as antibody precipitates occurring with rheumatoid arthritis or other autoimmune diseases. Chemical agents such as alpha-napthylisothiocyanate and chlorpromazine also can cause bile duct response and damage.

Hepatomegaly—Nonspecific enlargement of the liver caused by an increased hepatocyte growth rate (hyperplasia), hypertrophy, the lysosomal storage of undegraded cell metabolites in these cells and/or the proliferation of intracellular organelles such as the peroxisomes or endoplasmic reticulum. For example, phenobarbital and Wy-14,643 induce hyperplasia and hypertrophy, while Gaucher disease, resulting from a genetic deficiency in glucosyl cerebrosidase, leads to an enlarged liver due to hepatocyte glycolipid storage.

Fibrosis/Cirrhosis—Scar tissue replacement of the liver architecture, in general caused by long-term chronic xenobiotic insults such as alcohol abuse or viral infection. This form of liver response typically is preceded by a long period of chronic liver injury.

Primary hepatocellular carcinoma—Oncogenic transformation of a hepatocyte that begins at a single site and may rapidly expand and metastasize to surrounding tissues.

Given the high degree of variability in its causes and classifications, there currently is no specific measure of the liver's response to xenobiotics and biological agents. The following list outlines currently validated measures of liver homeostasis:

Nonintrusive assays including serum/plasma alanine aminotransferase (ALT), aspartate aminotransferase (ASP), alphafetoprotein (AFP) and gamma-glutamyltransferase (GGT) levels, as well as soft tissue imaging including sonography, magnetic resonance imaging and computed tomography, and finally, radioisotope metabolic labelling. Intrusive assays include needle biopsy and surgical or post mortem examination.

Typical measures of liver homeostasis are limited in one or more ways. For example, non-intrusive assays show poor correlation with liver histopathology and generally provide no prospective measure of how the liver will further change over time. Intrusive liver homeostasis assays present significant risk to the test subject. Therefore, they would not normally be employed unless the subject's life is already under serious threat, for example, from disease. Furthermore, intrusive assays require time-consuming and costly interpretation by expert pathologists, and may provide ambiguous results if the tissue changes are not homogeneous across the liver relative to the sample examined. Additionally, intrusive assays used in, for example, animal studies or animal models of disease, such as rats or mice, can produce tissue damage or death.

New human disease treatments must undergo extensive preclinical and regulatory testing in animals. Given the poor correlation of the available nonintrusive liver homeostasis assays with histopathology, the most intrusive assays are almost always employed in such preclinical and regulatory testing.

Therefore, there is a need for new liver response markers that can be used to screen candidate compounds for their ability to induce specific liver toxicity. Such new markers would enable liver toxicity to be detected at lower drug doses than is possible using conventional methods. In addition, use of the markers at early stages of the drug discovery process, could facilitate the ranking of candidate compounds according to their toxic effects. This would inevitably contribute to significant financial savings in the latter stages of the development process.

### SUMMARY OF THE INVENTION

The present invention provides methods and compositions for the identification of markers of liver response, for monitoring the effectiveness of liver response treatment, for selecting participants in clinical trials, for identifying patients most likely to have an adverse response to a particular therapeutic treatment and for use in the screening and development of drugs.

One aspect of the invention provides methods for identifying markers of liver response These methods are also suitable for drug screening and development, clinical screening, prognosis, monitoring the effects of therapy, and for identifying patients most likely to have an adverse response to a particular therapeutic treatment.

Another aspect of the invention provides methods for identification of liver response that comprise detecting in an appropriate sample, the presence or level of at least one Liver Response-Associated Protein Isoform (LRPI), e.g., one or more of the LRPIs disclosed herein or any combination thereof.

An additional aspect of the invention provides kits that may be used in the above recited methods and that may comprise single or multiple preparations of LRPI binding agents, together with other reagents, labels, substrates, if needed, and directions for use. The kits may be used for diagnosis of liver response, or may be used in assays for the identification of new diagnostic and/or therapeutic agents.

A further aspect of the invention provides binding agents to the LRPI nucleic acids or proteins, including for example, antisense nucleic acids, and antibodies.

Another aspect of the invention provides a preparation comprising an isolated LRPI, i.e., an LRPI substantially free from proteins or protein isoforms having a significantly different isoelectric point or a significantly different apparent molecular weight from the LRPI.

An additional aspect of the invention provides methods for identifying agents or compounds that can alleviate, block or otherwise modulate the toxicity, particularly hepatotoxicity, of drugs or therapies or otherwise offer protection to the liver from toxicity or damage caused or induced by drugs or therapies by analyzing the activity of one or more LRFs or LRPIs in the presence of a combination of said drugs or therapies with said agent or compound.

Another aspect of the invention provides for the use of a plurality of active agents in the preparation of a diagnostic array for clinical screening or diagnosis of liver response. Preferably the active agents bind to the DNA or RNA encoding an LRPI protein or are anti-LRPI antibodies. The active agents can be selected as diagnostic markers of specific liver response indications. Preferably the array will contain markers that are diagnostic for a plurality of indications of liver response.

Other objects and advantages will become apparent from a review of the ensuing detailed description taken in conjunction with the following illustrative drawings.

### BRIEF DESCRIPTION OF THE FIGURES

FIG. 1 is an image obtained from 2-dimensional electrophoresis of liver tissue lysate, which has been annotated to identify fifteen landmark features, designated RL1 to RL15, and which are illustrative of an embodiment of an aspect of the present invention.

FIG. 2 is an image obtained from 2-dimensional electrophoresis of serum, which has been annotated to identify ten landmark features, designated RDS1 to RDS10, and which are illustrative of an embodiment of an aspect of the present invention.

FIG. 3 is a flow chart depicting the characterization of an LRF and relationship of an LRF and LRPI(s). An LRF may be further characterized as or by an LRPI having a particular peptide sequence associated with its pI and MW. As depicted herein, an LRF may comprise one or more LRPI(s), which have indistinguishable pIs and MWs, but which have distinct peptide sequences. The peptide sequence(s) of the LRPI can be utilized to search database(s) for previously identified proteins comprising such peptide sequence(s), it can be ascertained whether a commercially available antibody exists which may recognize the previously identified protein and/or a member of its protein family.

### DETAILED DESCRIPTION OF THE INVENTION

The present invention provides methods and compositions useful, e.g. for clinical screening, diagnosis and prognosis of liver response in a mammalian subject, for monitoring the results of liver response therapy, for identifying patients most likely to have an adverse response to a particular therapeutic treatment and for drug screening and drug development. For clarity of disclosure, and not by way of limitation, the invention will be described with respect to the analysis of blood or liver tissue samples. However, as one skilled in the art will appreciate, based on the present description, the assays and techniques described herein can be applied to other types of samples, including a body fluid (e.g. blood or a fraction of blood comprising serum or plasma or both, spinal fluid, urine or saliva), a tissue sample from a subject at risk of having or developing a liver response (e.g. a biopsy such as a liver biopsy) or homogenate thereof.

For convenience and completeness, particular terms and phrases used in the following detailed description and claims are defined in the following section, as well as throughout the disclosure.

Definitions

The singular forms “a”, “an”, and “the” include plural references unless the context clearly dictates otherwise.

As used herein, an “aberrant level” means a level that is increased or decreased in a first sample compared with the level in a second sample from a subject free from breast cancer or a reference level.

“Agonist” refers to an agent that mimics or up-regulates (e.g., potentiates or supplements) the bioactivity of a polypeptide. An agonist may be a wild-type or derivative thereof having at least one bioactivity of the wild-type protein. An agonist may also be a compound that up-regulates expression of a gene or which increases at least one biological activity of a protein. An agonist may also be a compound which increases the interaction of a polypeptide with another molecule, e.g., a target peptide or nucleic acid.

“Antagonist” refers to an agent that down-regulates (e.g., suppresses or inhibits) at least one biological activity of a protein. An antagonist may be a compound which inhibits or decreases the interaction between a protein and another molecule, e.g., a target peptide or enzyme substrate. An antagonist may also be a compound that down-regulates expression of a gene or which reduces the amount of expressed protein present.

“Antibody” is intended to include whole antibodies, e.g., of any isotype (IgG, IgA, IgM, IgE, etc.), and includes fragments thereof which are also specifically reactive with a vertebrate, e.g., mammalian, protein. Antibodies may be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, humanized, or chimeric antibodies and other purified preparations of antibodies including antibody fragments and recombinant antibodies.

“Antisense” nucleic acid refers to oligonucleotides which specifically hybridize (e.g., bind) under cellular conditions with a nucleic acid, such as at the cellular mRNA and/or genomic DNA level, so as to inhibit expression of that gene, e.g., by inhibiting transcription and/or translation. The binding may be by conventional base pair complementarily, or, for example, in the case of binding to DNA duplexes, through specific interactions in the major groove of the double helix.

“Array” or “matrix” refer to an arrangement of addressable locations or “addresses” on a device. The locations may be arranged in two dimensional arrays, three dimensional arrays, or other matrix formats. The number of locations may range from several to at least hundreds of thousands. Most importantly, each location represents a totally independent reaction site. An “antibody array” referes to an array containing antibodies or antibody fragments. A “nucleic acid array” refers to an array containing nucleic acid probes, such as oligonucleotides or larger portions of genes. The nucleic acid on the array is preferably single stranded. Arrays wherein the probes are oligonucleotides are referred to as “oligonucelotide arrays” or “oligonucleotide chips” or “gene chips”. A “microarray”, also referred to as a “chip”, “biochip”, or “biological chip”, is an array of regions having a suitable density of discrete regions, e.g., of at least 100/cm², and preferably at least about 1000/cm². The regions in a microarray have dimensions, e.g. diameters, preferably in the range of between about 10-250 microns, and are separated from other regions in the array by the same distance.

“Biological activity” or “activity” which are used interchangeably, refer to an effector or antigenic function that is directly or indirectly performed by a polypeptide (whether in its native or denatured conformation), or by any subsequence thereof. Biological activities include binding to polypeptides, binding to other proteins or molecules, activity as a DNA binding protein, as a transcription regulator, ability to bind damaged DNA, etc. A biological activity may be modulated by directly affecting the subject polypeptide. Alternatively, a biological activity may be altered by modulating the level of the polypeptide, such as by modulating expression of the corresponding gene.

“Biological sample” or “sample”, refers to a sample obtained from an organism or from components (e.g., cells) of an organism. The sample may be of any biological tissue or fluid. Frequently the sample will be a “clinical sample” which is a sample derived from a patient. As used herein, a “biological sample” can be obtained from any source, including a body fluid sample such as serum, blood, blood cells (e.g., white cells), plasma, sputum, urine or a tissue sample (e.g. liver tissue sample) or fine needle biopsy samples, peritoneal fluid, and pleural fluid, or cells therefrom. “Blood” includes whole blood serum and plasma. Serum refers to the supernatant fluid produced by clotting and centrifugal sedimentation of a blood sample. Plasma refers to the supernatant fluid produced by inhibition of clotting (for example, by citrate or or a chelating agent such as EDTA) and centrifugal sedimentation of a blood sample. Whole blood refers to blood as collected, without particular separation on centrifugal sedimentation and includes any and/or all particular cellular, protein and lipid components of blood as well as serum.

Biological samples may also include sections of tissues such as frozen sections taken for histological purposes. The term “liver” or “liver tissue” as used herein refers to any portion of the tissue within the liver capsule, and further includes homogenates, extracts and fractions and cell lines derived therefrom.

“Biomarker” or “marker” refers to a biological molecule whose presence, concentration, activity, or post-translationally-modified state may be detected and correlated with the activity of a protein of interest.

“Chimeric Antibody” refers to a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397, which are incorporated herein by reference in their entirety.)

The term “constant region” as used herein, refers to the portion of the antibody molecule which confers effector functions. In the present invention, murine constant regions are substituted by human constant regions. The constant regions of the subject chimeric or humanized antibodies are derived from human immunoglobulins. The heavy chain constant region can be selected from any of the five isotypes: alpha, delta, epsilon, gamma or mu. Further, heavy chains of various subclasses (such as the IgG subclasses of heavy chains) are responsible for different effector functions and thus, by choosing the desired heavy chain constant region, antibodies with desired effector function can be produced. Preferred constant regions are gamma 1 (IgG1), gamma 3 (IgG3) and gamma 4 (IgG4). More preferred is an Fe region of the gamma 1 (IgG1) isotype. The light chain constant region can be of the kappa or lambda type, preferably of the kappa type. In one embodiment the light constant chain is the human kappa constant chain (Heiter et al. (1980) Cell 22:197-207) and the heavy constant chain is the human IgG1 constant chain (Ellison et al. (1982) Nucleic Acids Res. 10:4076-4079).

“Derivative” refers to a polypeptide that comprises an amino acid sequence of a second polypeptide which has been altered by the introduction of amino acid residue substitutions, deletions or additions. The derivative polypeptide possess a similar or identical function as the second polypeptide.

“Diagnosis” refers to diagnosis, prognosis, monitoring, characterizing, selecting patients, including participants in clinical trials, and identifying patients at risk for or having a particular disorder or those most likely to have an adverse response to a particular therapeutic treatment, or for assessing or monitoring a patient's response to a particular therapeutic treatment.

“Feature” refers to a spot detected in a 2D gel, and the term “Liver Response-Associated Feature” (LRF) refers to a feature that is differentially present in a sample (e.g. a sample of blood or liver) from a subject having liver response compared with a sample (e.g. a sample of blood or liver) from a subject relatively free from liver response. A feature or spot detected in a 2D gel is characterized by its isoelectric point (pI) and molecular weight (MW) as determined by 2D gel electrophoresis. As used herein, a feature is “differentially present” in a first sample with respect to a second sample when a method for detecting the said feature (e.g., 2D electrophoresis) gives a different signal when applied to the first and second samples. An LRF, (or a protein isoform, i.e. LRPI, as defined infra) is “increased” in the first sample with respect to the second if the method of detection indicates that the LRF or LRPI is more abundant in the first sample than in the second sample, or if the LRF or LRPI is detectable in the first sample and substantially undetectable in the second sample, or if the LRF or LRPI is more frequently detectable in the first sample than in the second sample. Conversely, an LRF or LRPI is “decreased” in the first sample with respect to the second if the method of detection indicates that the LRF or LRPI is less abundant in the first sample than in the second sample, or if the LRF or LRPI is undetectable in the first sample and detectable in the second sample, or if the LRF or LRPI is detected less frequently in the first sample than in the second sample.

Particularly, the relative abundance of a feature in two samples is determined in reference to its normalized signal, in two steps. First, the signal obtained upon detecting the feature in a sample is normalized by reference to a suitable background parameter, e.g., (a) to the total protein in the sample being analyzed (e.g., total protein loaded onto a gel); (b) to an Expression Reference Feature (ERF) i.e., a feature whose abundance is substantially invariant, within the limits of variability of the Preferred Technology, in the population of subjects being examined, e.g. the ERFs disclosed below, or (c) more preferably to the total signal detected as the sum of each of all proteins in the sample.

Secondly, the normalized signal for the feature in one sample or sample set is compared with the normalized signal for the same feature in another sample or sample set in order to identify features that are “differentially present” in the first sample (or sample set) with respect to the second.

“Fold change” includes “fold increase” and “fold decrease” and refers to the relative increase or decrease in abundance of an LRF or the relative increase or decrease in expression or activity of a polypeptide (e.g. an LRPI, as defined infra.) in a first sample or sample set compared to a second sample (or sample set). An LRF or polypeptide fold change may be measured by any technique known to those of skill in the art, albeit the observed increase or decrease will vary depending upon the technique used. Preferably, fold change is determined herein as described in the Examples herein.

“Fragment” refers to a peptide or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of a second polypeptide. The fragment of an LRPI may or may not possess a functional activity of the a second polypeptide.

The term “humanised antibody”, as used herein, refers to an antibody derived from a non-human antibody, typically murine, that retains or substantially retains the antigen-binding properties of the parent antibody but which is less immunogenic in humans. A “humanised antibody” typically has one or more complementary determining regions (CDRs) from the antibody of a non-human species and a framework region from a human immunoglobulin molecule.

The term “complementarity determining region” (CDR), as used herein, refers to amino acid sequences which together define the binding affinity and specificity of the natural Fv region of a native immunoglobulin binding site as delineated by Kabat et al., Sequence of Proteins of Immunological Interest, 5^{th} Edition, The United States Department of Health and Human Services, The United States Government Printing Office, 1991.

The term “framework region” (FR), as used herein, refers to amino acid sequences interposed between CDRs. These portions of the antibody serve to hold the CDRs in appropriate orientation (allows for CDRs to bind antigen).

“Interact” is meant to include detectable interactions between molecules, such as may be detected using, for example, a hybridization assay. Interact also includes “binding” interactions between molecules. Interactions may be, for example, protein-protein, protein-nucleic acid, protein-small molecule or small molecule-nucleic acid in nature.

“Isolated”, with respect to nucleic acids, such as DNA or RNA, refers to molecules separated from other DNAs, or RNAs, respectively, that are present in the natural source of the macromolecule. Isolated also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. “Isolated” also refers to polypeptides which are isolated from other cellular proteins and is meant to encompass both purified and recombinant polypeptides.

As used herein, an LRPI is “isolated” when it is present in a preparation that is substantially free of contaminating proteins, i.e., a preparation in which less than 10% (preferably less than 5%, more preferably less than 1%) of the total protein present is contaminating protein(s)

“Liver response” refers to and includes the activities and consequent alterations in liver function and other organ or cellular function and any condition that comes about from interaction of the liver with exogenous agents, including xenobiotics, viruses or other biological agents, particularly those agents with are hepatotoxic, which can generate hepatocellular necrosis, hepatomegaly, hepatocellular carcinoma or which otherwise reduce or alter the function or physiological response of the liver. Liver response encompasses and includes those activities, alterations and physiological occurrances in the liver, or otherwise associated with the liver or the liver's function, which take place during any alteration of the liver including but not limited to any aspect or phase of detoxification pathway modulation, staetosis, hepatocellular necrosis, bile duct response/cholestasis, hepatomegaly, fibrosis/cirrhosis, and hepatocellular carcinoma. Xenobiotic agents include but are not limited to chemical poisoning, chemotherapeutics and environmental agents, including for example, acetaminophen, alpha-naphthylisothiocyanate (ANIT), phenobarbital, Wy-14,643 and valproic acid, as particularly utilized herein.

“Liver response” refers to and includes the activities and consequent alterations in liver function and other organ or cellular function and any condition that comes about from interaction of the liver with exogenous agents, including xenobiotics, viruses or other biological agents, particularly those agents with are hepatotoxic, which can generate hepatocellular necrosis, hepatomegaly, hepatocellular carcinoma or which otherwise reduce or alter the function or physiological response of the liver. “Liver response” encompasses and includes those activities, alterations and physiological occurrances in the liver, or otherwise associated with the liver or the liver's function, which take place during any alteration of the liver including but not limited to any aspect or phase of detoxification pathway modulation, staetosis, hepatocellular necrosis, bile duct response/cholestasis, hepatomegaly, fibrosis/cirrhosis, hepatocellular necrosis and hepatocellular carcinoma.

“Liver Response-Associated Protein Isoform” (LRPI) refers to a polypeptide that is differentially present in a first sample or sample set from a subject having liver response compared with a second sample or sample set from a subject free from liver response. As used herein, an LRPI is “differentially present” in a first sample or sample set with respect to a second sample or sample set when a method for detecting the said feature, (e.g., 2D electrophoresis or immunoassay) gives a different signal when applied to the first and second samples or sample sets (as described above in relation to LRFs). An LRPI is characterised by one or more peptide sequences of which it is comprised, and further by a pI and MW, preferably determined by 2D electrophoresis.

“LRPI analog” refers to a polypeptide that possesses a similar or identical function as an LRPI but need not necessarily comprise an amino acid sequence that is similar or identical to the amino acid sequence of the LRPI, or possess a structure that is similar or identical to that of the LRPI. As used herein, an amino acid sequence of a polypeptide is “similar” to that of an LRPI if it satisfies at least one of the following criteria: (a) the polypeptide has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of the LRPI; (b) the polypeptide is encoded by a nucleotide sequence that hybridizes under stringent conditions (preferably highly and including moderately stringent conditions) to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the LRPI; or (c) the polypeptide is encoded by a nucleotide sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleotide sequence encoding the LRPI. “Highly stringent conditions” refers to hybridisation to filter-bound DNA in 0.5 M NaHPO₄, 7% sodium dodecyl sulfate (SDS), 1 mM EDTA at 65 C., and washing in 0.1× SSC/0.1% SDS at 68 C. (Ausubel F. M. et al., eds., 1989, Current Protocols in Molecular Biology, Vol. I, Green Publishing Associates, Inc., and John Wiley & Sons, Inc., New York, at p. 2.10.3)

For some applications, less stringent conditions for duplex formation are required. As used herein “moderately stringent conditions” refers to washing in 0.2× SSC/0.1% SDS at 42 C. (Ausubel et al., 1989, supra).

As used herein, a polypeptide with “similar structure” to that of an LRPI refers to a polypeptide that has a similar secondary, tertiary or quarternary structure as that of the LRPI. The structure of a polypeptide can be determined by methods known to those skilled in the art, including, for example, X-ray crystallography, nuclear magnetic resonance, and crystallographic electron microscopy.

“LRPI binding agent” or simply “binding agent” refers to a molecule capable of interacting with (binding to) an LRPI nucleic acid or polypeptide. Such a molecule may include but not be limited to an antisense nucleic acid, an antibody, ligand, or other polypeptide, polynucleotide, hormone, small molecule or virus.

“LRPI fusion protein” refers to a polypeptide that comprises (i) an amino acid sequence of an LRPI, an LRPI fragment, an LRPI-related polypeptide or a fragment of an LRPI-related polypeptide and (ii) an amino acid sequence of a heterologous polypeptide (i.e., a non-LRPI, non-LRPI fragment or non-LRPI-related polypeptide).

“LRPI homolog” refers to a polypeptide that comprises an amino acid sequence similar to that of an LRPI but does not necessarily possess a similar or identical function as the LRPI.

“LRPI ortholog” refers to a non-rat polypeptide that (i) comprises an amino acid sequence similar to that of an LRPI and (ii) possesses a similar or identical function to that of the LRPI.

“LRPI-related polypeptide” refers to an LRPI homolog, an LRPI analog, an isoform of LRPI, an LRPI ortholog, or any combination thereof.

“Modulate” in reference to expression or activity of an LRF, LRPI or an LRPI-related polypeptide refers to any change, e.g., upregulation (i.e., inhibition or suppression) or downregulation (i.e., activation or stimulation), increase or decrease, of the expression or activity of the LRF, LRPI or LRPI-related polypeptide. Those skilled in the art, based on the present disclosure, will understand that such modulation can be determined by assays known to those of skill in the art.

As used herein, the terms “MW” and “pI” are defined, respectively, to mean the apparent molecular weight and the apparent isoelectric point of a feature or protein isoform as measured in exact accordance with the Reference Protocol identified in Section 6 below.

“Nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

The “percent identity” of two amino acid sequences or of two nucleic acid sequences can be or is generally determined by aligning the sequences for optimal comparison purposes (e.g., gaps can be introduced in either sequences for best alignment with the other sequence) and comparing the amino acid residues or nucleotides at corresponding positions. The “best alignment” is an alignment of two sequences that results in the highest percent identity. The percent identity is determined by the number of identical amino acid residues or nucleotides in the sequences being compared (i.e., % identity=# of identical positions/total # of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov.

Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti (1994) Comput. Appl. Biosci., 10 :3-5; and FASTA described in Pearson and Lipman (1988) Proc. Natl. Acad. Sci. 85:2444-8. Within FASTA, ktup is a control option that sets the sensitivity and speed of the search.

“Prophylactic” or “therapeutic” treatment refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

“Protein”, “polypeptide” and “peptide” are used interchangeably herein when referring to a gene product, e.g., as may be encoded by a coding sequence. By “gene product” it is meant a molecule that is produced as a result of transcription of a gene. Gene products include RNA molecules transcribed from a gene, as well as proteins translated from such transcripts.

“Recombinant protein”, “heterologous protein” and “exogenous protein” are used interchangeably to refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid.

“Small molecule” refers to a composition, which has a molecular weight of less than about 1000 kDa. Small molecules may be nucleic acids, peptides, polypeptides, peptidomimetics, carbohydrates, lipids or other organic (carbon-containing) or inorganic molecules. As those skilled in the art will appreciate, based on the present description, libraries of chemical and/or biological extensive libraries of chemical and/or biological mixtures, often fungal, bacterial, or algal extracts, may be screened with any of the assays of the invention to identify compounds that modulate a bioactivity.

“Therapeutic agent” or “therapeutic” refers to an agent capable of having a desired biological effect on a host. Chemotherapeutic and genotoxic agents are examples of therapeutic agents that are generally known to be chemical in origin, as opposed to biological, or cause a therapeutic effect by a particular mechanism of action, respectively. Examples of therapeutic agents of biological origin include growth factors, hormones, and cytokines. A variety of therapeutic agents are known in the art and may be identified by their effects. Certain therapeutic agents are capable of regulating red cell proliferation and differentiation. Examples include chemotherapeutic nucleotides, drugs, hormones, non-specific (non-antibody) proteins, oligonucleotides (e.g., antisense oligonucleotides that bind to a target nucleic acid sequence (e.g., mRNA sequence)), peptides, and peptidomimetics.

“Treatment” refers to therapy, prevention and prophylaxis and particularly refers to the administration of medicine or the performance of medical procedures with respect to a patient, for either prophylaxis (prevention) or to cure or reduce the extent of the infirmity or malady in the instance where the patient is afflicted.

“Therapeutic effect” refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

“Treating” a disease in a subject or “treating” a subject having a disease refers to subjecting the subject to a pharmaceutical treatment, e.g., the administration of a drug, such that at least one symptom of the disease is decreased or prevented.

“Two-dimensional electrophoresis” (2D-electrophoresis) means a technique comprising denaturing electrophoresis, followed by isoelectric focusing; this generates a two-dimensional gel (2D-gel) containing a plurality of separated proteins.

“Variant,” when used in the context of a polynucleotide sequence, may encompass a polynucleotide sequence related to that of gene X or the coding sequence thereof. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one base. The presence of SNPs may be indicative of, for example, a certain population, a disease state, or a propensity for a disease state.

A “variant” of polypeptide X refers to a polypeptide having the amino acid sequence of peptide X in which is altered in one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological or immunological activity may be found using computer programs well known in the art, for example, LASERGENE software (DNASTAR).

Such variants can differ in their amino acid composition (e.g. as a result of allelic or natural variation in the amino acid sequence, e.g. as a result of alternative mRNA or pre-mRNA processing, e.g. alternative splicing or limited proteolysis) and in addition, or in the alternative, may arise from differential post-translational modification (e.g., glycosylation, acylation, phosphorylation), particularly as a result of differences in their amino acid composition.

Identifying Liver Response Associated Features

As described in detail in the following Examples, two-dimensional electrophoresis was used to generate a two-dimensional gel (2D-gel) containing a plurality of separated proteins. Preferably, the step of denaturing electrophoresis uses polyacrylamide electrophoresis in the presence of sodium dodecyl sulfate (SDS-PAGE). Especially preferred are the highly accurate and automatable methods and apparatuses described in PCT Publication No WO 98/23950 and in U.S. Pat. No. 6,064,754, each of which is incorporated herein by reference in its entirety with particular reference to the experimental protocol. Briefly, these methods and apparatuses provide efficient, computer-assisted means for identifying, selecting and characterizing biomolecules (e.g. proteins, including glycoproteins) in a biological sample. A two-dimensional array is generated by separating biomolecules on a two-dimensional gel according to their electrophoretic mobility and isoelectric point. A computer-generated digital profile of the array is generated, representing the identity, apparent molecular weight, isoelectric point, and relative abundance of a plurality of biomolecules detected in the two-dimensional array, thereby permitting computer-mediated comparison of profiles from multiple biological samples, as well as computer aided excision of separated proteins of interest.

A particular scanner for detecting fluorescently labeled proteins is described in WO 96/36882 and in the Ph.D. thesis of David A. Basiji, entitled “Development of a High-throughput Fluorescence Scanner Employing Internal Reflection Optics and Phase-sensitive Detection (Total Internal Reflection, Electrophoresis)”, University of Washington (1997), Volume 58/12-B of Dissertation Abstracts International, page 6686, the contents of each of which are incorporated herein by reference. These documents describe an image scanner designed specifically for automated, integrated operation at high speeds. The scanner can image gels that have been stained with fluorescent dyes or silver stains, as well as storage phosphor screens. The Basiji thesis provides a phase-sensitive detection system for discriminating modulated fluorescence from baseline noise due to laser scatter or homogeneous fluorescence, but the scanner can also be operated in a non-phase-sensitive mode. This phase-sensitive detection capability would increase the sensitivity of the instrument by an order of magnitude or more compared to conventional fluorescence imaging systems. The increased sensitivity would reduce the sample-preparation load on the upstream instruments while the enhanced image quality simplifies image analysis downstream in the process.

A more highly preferred scanner is the Apollo 2 scanner (Oxford Glycosciences, Oxford, UK), which is a modified version of the above described scanner. In the Apollo 2 scanner, the gel is transported through the scanner on a precision lead-screw drive system. This is preferable to laying the glass plate on the belt-driven system that is described in the Basiji thesis, as it provides a reproducible means of accurately transporting the gel past the imaging optics.

In the Apollo 2 scanner, the gel is secured against three alignment stops that rigidly hold the glass plate in a known position. By doing this in conjunction with the above precision transport system, the absolute position of the gel can be predicted and recorded. This ensures that co-ordinates of each feature on the gel can be determined more accurately and communicated, if desired, to a cutting robot for excision of the feature. In the Apollo 2 scanner, the carrier that holds the gel has four integral fluorescent markers for use to correct the image geometry. These markers are a quality control feature that confirms that the scanning has been performed correctly.

In comparison to the scanner described in the Basiji thesis, the optical components of the Apollo 2 scanner have been inverted. In the Apollo 2 scanner, the laser, mirror, waveguide and other optical components are above the glass plate being scanned. The scanner described in the Basiji thesis has these components underneath. In the Apollo 2 scanner, the glass plate is mounted onto the scanner gel side down, so that the optical path remains through the glass plate. By doing this, any particles of gel that may break away from the glass plate will fall onto the base of the instrument rather than into the optics. This does not affect the functionality of the system, but increases its reliability.

Still more preferred is the Apollo 3 scanner, in which the signal output is digitized to the full 16-bit data without any peak saturation or without square root encoding of the signal. A compensation algorithm has also been applied to correct for any variation in detection sensitivity along the path of the scanning beam. This variation is due to anomalies in the optics and differences in collection efficiency across the waveguide. A calibration is performed using a perspex plate with an even fluorescence throughout. The data received from a scan of this plate are used to determine the multiplication factors needed to increase the signal from each pixel level to a target level. These factors are then used in subsequent scans of gels to remove any internal optical variations.

Liver Response-Associated Features (LRFS)

The LRFs disclosed herein have been identified by comparing blood or liver samples taken from subjects with liver response against blood or liver samples from subjects substantially free from liver response. Subjects substantially free from liver response include subjects with no known disease or condition (normal subjects) and subjects with diseases or conditions other than liver response and subjects who have not been administered or otherwise exposed to a heptotoxic, xenobiotic or other exogenous agent which does or may elicit a liver response. Comparisons were made between subjects with a liver response at three different time points as described in the Examples infra., herein designated “early liver response,” “full manifestation liver response” and/or “late liver response.” Early, full manifestation and late liver response correspond experimentally to time periods after which a hepatotoxic or other exogenous agent has been administered or time periods after which an individual subject has been exposed to a hepatotoxic, xenobiotic or other exogenous agent which precipitates a liver response. In addition or alternative to a time period, the extent of liver response, and thus whether response is early, full or late, can also be determined or assessed by evaluating liver function through clinical pathology, laboratory data including markers already known in the art including, for instance, ALT, ASP, AFP and GGT, and histopathology. In general, early liver response refers to the initial liver response shortly after administration or exposure. Full manifestation liver response refers to fulminant response where alterations in liver activity, liver function and pathology are significant and some time has passed following administration or exposure. Late liver response refers to the response seen a substantial time after administration or exposure, and can refer particularly to a liver response due to recovery from such administration or exposure.

Twelve groups of LRFs have been identified through the methods and apparatuses described herein.. The first group consists of LRFs that are decreased in the liver tissue of subjects having early liver response as compared with the tissue of subjects free from liver response. These LRFs can be described by apparent molecular weight (MW) and isoelectric point (pI) as provided in Table I.

TABLE I
LRFs Decreased in Liver Tissue of Subjects Having Early Liver Response

| | Relative | | | |
|---|---|---|---|---|
| | Abundance | | | P value |
| | (foreground over | | | (Rank Sum |
| LRF# | background) | pI | MW (Da) | Test) |
| LRF-1 | −4.87 | 8.95 | 16081 | 0.0075 |
| LRF-2 | −1.42 | 4.60 | 62363 | 0.0088 |
| LRF-3 | −5.87 | 6.59 | 12894 | 0.0075 |
| LRF-4 | −4.37 | 4.93 | 26181 | 0.0073 |
| LRF-386 | −4.23 | 5.01 | 27830 | 0.0075 |
| LRF-387 | −6.63 | 5.44 | 24451 | 0.0075 |

The second group consists of LRFs that are increased in the tissue of subjects having early liver response as compared with the tissue of subjects free from liver response, as this term is defined above. These LRFs can be described by apparent MW pI as provided in Table II.

Table II. LRFs Increased in Liver Tissue of Subjects Having Early Liver Response

| | Relative | | | |
|---|---|---|---|---|
| | Abundance | | | P value |
| | (foreground over | | | (Rank Sum |
| LRF# | background) | pI | MW (Da) | Test) |
| LRF-6 | 1.77 | 8.36 | 31027 | 0.0122 |
| LRF-8 | 1.25 | 5.57 | 23697 | 0.0122 |
| LRF-9 | 1.40 | 4.79 | 37664 | 0.0196 |
| LRF-10 | 2.13 | 7.73 | 123358 | 0.0122 |
| LRF-11 | 1.53 | 6.25 | 79681 | 0.0119 |
| LRF-12 | 8.89 | 7.82 | 60454 | 0.0254 |
| LRF-13 | 2.32 | 5.78 | 11799 | 0.0122 |
| LRF-14 | 2.01 | 4.99 | 56166 | 0.0200 |
| LRF-15 | 2.47 | 9.38 | 80495 | 0.0122 |
| LRF-16 | 1.45 | 5.81 | 30167 | 0.0122 |
| LRF-17 | 1.61 | 5.81 | 30167 | 0.0212 |
| LRF-18 | 5.25 | 5.46 | 40000 | 0.0254 |
| LRF-19 | 1.48 | 5.83 | 22631 | 0.0119 |
| LRF-20 | 2.60 | 6.35 | 28978 | 0.0075 |
| LRF-21 | 1.50 | 8.25 | 35532 | 0.0122 |
| LRF-22 | 1.41 | 9.80 | 11450 | 0.0122 |
| LRF-23 | 1.90 | 8.06 | 31812 | 0.0122 |
| LRF-24 | 3.34 | 10.76 | 23549 | 0.0075 |
| LRF-25 | 1.43 | 4.80 | 26633 | 0.0122 |
| LRF-26 | 1.33 | 5.28 | 58041 | 0.0216 |
| LRF-27 | 1.22 | 7.31 | 124197 | 0.0114 |
| LRF-28 | 1.53 | 5.65 | 49698 | 0.0119 |
| LRF-29 | 1.57 | 6.19 | 77203 | 0.0122 |
| LRF-30 | 2.08 | 4.73 | 57645 | 0.0112 |
| LRF-31 | 2.07 | 8.97 | 27694 | 0.0119 |
| LRF-32 | 1.47 | 5.63 | 77203 | 0.0119 |
| LRF-36 | 1.33 | 5.73 | 74823 | 0.0097 |
| LRF-37 | 3.98 | 7.59 | 33242 | 0.0254 |
| LRF-38 | 2.09 | 5.02 | 56260 | 0.0216 |
| LRF-389 | 1.15 | 9.76 | 18844 | 0.0465 |
| LRF-390 | 5.03 | 6.36 | 46232 | 0.0073 |
| LRF-391 | 1.66 | 6.97 | 28172 | 0.0119 |
| LRF-392 | 1.29 | 7.98 | 29700 | 0.0122 |
| LRF-393 | 1.51 | 8.88 | 28876 | 0.0122 |
| LRF-394 | 1.17 | 6.50 | 25990 | 0.0459 |
| LRF-395 | 1.97 | 9.74 | 25549 | 0.0119 |
| LRF-397 | 1.89 | 5.74 | 68828 | 0.0122 |
| LRF-399 | 1.82 | 5.83 | 67405 | 0.0367 |

The third group consists of LRFs that are decreased in the tissue of subjects having full manifestation liver response as compared with the tissue of subjects free from liver response. These LRFs can be described by apparent MW and pI as provided in Table III.

Table III. LRFs Decreased in Liver Tissue of Subjects Having Full Manifestation Liver Response

| | Relative | | | |
|---|---|---|---|---|
| | Abundance | | | P value |
| | (foreground over | | | (Rank Sum |
| LRF# | background) | pI | MW (Da) | Test) |
| LRF-53 | −5.83 | 6.14 | 72138 | 0.0075 |
| LRF-54 | −2.33 | 4.81 | 51921 | 0.0097 |
| LRF-56 | −3.20 | 6.05 | 45895 | 0.0073 |
| LRF-386 | −4.23 | 5.01 | 27830 | 0.0075 |
| LRF-387 | −6.63 | 5.44 | 24451 | 0.0075 |
| LRF-400 | −17.27 | 8.84 | 11701 | 0.0075 |

The fourth group consists of LRFs that are increased in the tissue of subjects having full manifestation liver response as compared with the tissue of subjects free from liver response. These LRFs can be described by apparent MW and pI as provided in Table IV.

Table IV. LRFs Increased in Liver Tissue of Subjects Having Full Manifestation Liver Response

| | Relative | | | |
|---|---|---|---|---|
| | Abundance | | | P value |
| | (foreground over | | | (Rank Sum |
| LRF# | background) | pI | MW (Da) | Test) |
| LRF-61 | 3.09 | 8.08 | 14573 | 0.0119 |
| LRF-63 | 1.45 | 6.01 | 46782 | 0.0119 |
| LRF-64 | 5.37 | 5.87 | 58635 | 0.0075 |
| LRF-66 | 1.93 | 5.44 | 24451 | 0.0345 |
| LRF-67 | 3.32 | 4.90 | 33525 | 0.0119 |
| LRF-69 | 1.32 | 7.67 | 40789 | 0.0122 |
| LRF-70 | 1.50 | 9.50 | 11592 | 0.0160 |
| LRF-71 | 1.35 | 7.80 | 27225 | 0.0122 |
| LRF-72 | 1.90 | 4.69 | 54509 | 0.0117 |
| LRF-73 | 1.15 | 7.82 | 55445 | 0.0117 |
| LRF-74 | 1.42 | 6.70 | 92196 | 0.0361 |
| LRF-75 | 1.36 | 7.58 | 30261 | 0.0200 |
| LRF-76 | 1.61 | 5.24 | 52175 | 0.0153 |
| LRF-77 | 1.39 | 7.04 | 22559 | 0.0122 |
| LRF-78 | 1.95 | 6.36 | 32515 | 0.0122 |
| LRF-79 | 1.34 | 7.52 | 24017 | 0.0200 |
| LRF-80 | 1.40 | 5.12 | 127612 | 0.0216 |
| LRF-81 | 1.57 | 6.61 | 41576 | 0.0119 |
| LRF-83 | 1.40 | 5.00 | 15334 | 0.0367 |
| LRF-84 | 1.57 | 9.26 | 52497 | 0.0119 |
| LRF-85 | 1.72 | 6.29 | 128480 | 0.0122 |
| LRF-87 | 1.37 | 5.59 | 27762 | 0.0216 |
| LRF-88 | 1.50 | 6.21 | 23277 | 0.0122 |
| LRF-89 | 1.43 | 6.32 | 40144 | 0.0157 |
| LRF-90 | 1.47 | 5.14 | 106705 | 0.0216 |
| LRF-91 | 1.18 | 6.21 | 39258 | 0.0117 |
| LRF-92 | 1.47 | 6.51 | 59855 | 0.0117 |
| LRF-389 | 1.15 | 9.76 | 18844 | 0.0465 |
| LRF-390 | 5.03 | 6.36 | 46232 | 0.0073 |
| LRF-391 | 1.66 | 6.97 | 28172 | 0.0119 |
| LRF-392 | 1.29 | 7.98 | 29700 | 0.0122 |
| LRF-393 | 1.51 | 8.88 | 28876 | 0.0122 |
| LRF-394 | 1.17 | 6.50 | 25990 | 0.0459 |
| LRF-395 | 1.97 | 9.74 | 25549 | 0.0119 |
| LRF-397 | 1.89 | 5.74 | 68828 | 0.0122 |
| LRF-399 | 1.82 | 5.83 | 67405 | 0.0367 |
| LRF-401 | 1.30 | 7.13 | 39509 | 0.0119 |
| LRF-402 | 1.19 | 7.37 | 51294 | 0.0122 |
| LRF-403 | 1.55 | 7.83 | 51921 | 0.0367 |
| LRF-405 | 1.40 | 6.76 | 30546 | 0.0278 |
| LRF-407 | 1.62 | 6.04 | 25363 | 0.0119 |
| LRF-409 | 1.29 | 5.59 | 27762 | 0.0119 |
| LRF-410 | 1.31 | 10.23 | 38026 | 0.0345 |

The fifth group consists of LRFs that are decreased in the tissue of subjects having late liver response as compared with the tissue of subjects free from liver response. These LRFs can be described by apparent MW and pI as provided in Table V.

Table V. LRFs Decreased in Liver Tissue of Subjects Having Late Liver Response

| | Relative | | | |
|---|---|---|---|---|
| | Abundance | | | P value |
| | (foreground over | | | (Rank Sum |
| LRF# | background) | pI | MW (Da) | Test) |
| LRF-107 | −1.25 | 4.66 | 13804 | 0.0097 |
| LRF-400 | −17.27 | 8.84 | 11701 | 0.0075 |

The sixth group consists of LRFs that are increased in the tissue of subjects having late liver response as compared with the tissue of subjects free from liver response. These LRFs can be described by apparent MW and pI as provided in Table VI.

Table VI. LRFs Increased in Liver Tissue of Subjects Having Late Liver Response

| | Relative | | | |
|---|---|---|---|---|
| | Abundance | | | P value |
| | (foreground over | | | (Rank Sum |
| LRF# | background) | pI | MW (Da) | Test) |
| LRF-109 | 1.33 | 8.13 | 64092 | 0.0216 |
| LRF-110 | 2.33 | 6.19 | 52175 | 0.0216 |
| LRF-111 | 1.41 | 5.57 | 23697 | 0.0200 |
| LRF-112 | 5.38 | 4.99 | 49643 | 0.0075 |
| LRF-113 | 1.31 | 5.84 | 45749 | 0.0119 |
| LRF-114 | 1.45 | 6.10 | 183101 | 0.0119 |
| LRF-115 | 3.16 | 5.85 | 162581 | 0.0075 |
| LRF-119 | 1.68 | 6.86 | 46042 | 0.0212 |
| LRF-120 | 1.33 | 8.25 | 35532 | 0.0216 |
| LRF-121 | 1.24 | 5.51 | 54245 | 0.0367 |
| LRF-122 | 1.61 | 9.01 | 11379 | 0.0216 |
| LRF-123 | 1.73 | 4.63 | 11809 | 0.0119 |
| LRF-124 | 1.71 | 6.27 | 27026 | 0.0122 |
| LRF-126 | 1.96 | 5.67 | 61937 | 0.0216 |
| LRF-127 | 1.89 | 5.02 | 49336 | 0.0122 |
| LRF-128 | 1.93 | 5.78 | 54377 | 0.0112 |
| LRF-129 | 1.33 | 5.28 | 58041 | 0.0119 |
| LRF-130 | 1.36 | 6.21 | 57505 | 0.0278 |
| LRF-131 | 1.10 | 5.16 | 12722 | 0.0200 |
| LRF-132 | 1.25 | 6.30 | 66705 | 0.0216 |
| LRF-133 | 1.60 | 6.30 | 27762 | 0.0122 |
| LRF-134 | 5.62 | 6.01 | 57365 | 0.0075 |
| LRF-395 | 1.97 | 9.74 | 25549 | 0.0119 |
| LRF-399 | 1.82 | 5.83 | 67405 | 0.0367 |
| LRF-401 | 1.30 | 7.13 | 39509 | 0.0119 |
| LRF-402 | 1.19 | 7.37 | 51294 | 0.0122 |
| LRF-403 | 1.55 | 7.83 | 51921 | 0.0367 |
| LRF-405 | 1.40 | 6.76 | 30546 | 0.0278 |
| LRF-407 | 1.62 | 6.04 | 25363 | 0.0119 |
| LRF-409 | 1.29 | 5.59 | 27762 | 0.0119 |
| LRF-410 | 1.31 | 10.23 | 38026 | 0.0345 |

The seventh group consists of LRFs that are decreased in the blood of subjects with early liver response as compared with the blood of subjects free from liver response. These LRFs can be described by apparent MW and pI as provided in Table VII.

Table VII. LRFs Decreased in Blood of Subjects Having Early Liver Response

| | Relative | | | |
|---|---|---|---|---|
| | Abundance | | | P value |
| | (foreground over | | | (Rank Sum |
| LRF# | background) | pI | MW (Da) | Test) |
| LRF-156 | −1.38 | 4.93 | 54483 | 0.0216 |
| LRF-157 | −1.28 | 6.17 | 28245 | 0.0122 |
| LRF-158 | −1.33 | 5.89 | 128039 | 0.0119 |
| LRF-159 | −1.51 | 6.09 | 138959 | 0.0122 |
| LRF-160 | −2.62 | 5.09 | 42125 | 0.0097 |
| LRF-161 | −4.38 | 4.94 | 36692 | 0.0075 |
| LRF-162 | −2.10 | 4.95 | 35503 | 0.0122 |
| LRF-163 | −2.29 | 5.13 | 31133 | 0.0122 |
| LRF-164 | −1.56 | 5.31 | 40165 | 0.0122 |
| LRF-165 | −4.04 | 5.88 | 27988 | 0.0075 |
| LRF-166 | −3.22 | 5.65 | 38289 | 0.0122 |
| LRF-167 | −1.76 | 4.89 | 49621 | 0.0122 |
| LRF-168 | −2.49 | 5.10 | 73017 | 0.0122 |
| LRF-169 | −8.33 | 5.62 | 45452 | 0.0075 |
| LRF-170 | −1.90 | 6.03 | 103852 | 0.0122 |
| LRF-172 | −1.67 | 5.47 | 69633 | 0.0119 |
| LRF-173 | −1.83 | 6.38 | 68285 | 0.0119 |
| LRF-174 | −1.78 | 6.37 | 73744 | 0.0122 |
| LRF-175 | −2.33 | 5.40 | 86193 | 0.0122 |
| LRF-176 | −1.25 | 4.62 | 51617 | 0.0160 |
| LRF-177 | −1.53 | 7.08 | 19005 | 0.0160 |
| LRF-178 | −1.38 | 7.54 | 104678 | 0.0097 |
| LRF-179 | −1.37 | 6.84 | 108902 | 0.0212 |
| LRF-181 | −6.05 | 6.06 | 43714 | 0.0075 |
| LRF-182 | −2.81 | 5.76 | 68203 | 0.0178 |
| LRF-183 | −1.63 | 5.39 | 103579 | 0.0160 |
| LRF-184 | −1.96 | 7.55 | 77168 | 0.0122 |

The eighth group consists of LRFs that are increased in the blood of subjects with early liver response as compared with the blood of subjects free from liver response. These LRFs can be described by apparent MW and pI as provided in Table VIII.

Table VIII. LRFs Increased in Blood of Subjects Having Early Liver Response

| | Relative | | | |
|---|---|---|---|---|
| | Abundance | | | P value |
| | (foreground over | | | (Rank Sum |
| LRF# | background) | pI | MW (Da) | Test) |
| LRF-217 | 2.16 | 5.06 | 44232 | 0.0122 |
| LRF-218 | 1.35 | 5.42 | 23154 | 0.0122 |
| LRF-219 | 1.42 | 4.95 | 34894 | 0.0122 |
| LRF-220 | 1.32 | 6.99 | 78836 | 0.0367 |
| LRF-221 | 2.07 | 5.29 | 38416 | 0.0122 |
| LRF-222 | 1.45 | 4.80 | 37589 | 0.0122 |
| LRF-223 | 1.34 | 7.43 | 28246 | 0.0117 |
| LRF-224 | 1.30 | 7.14 | 28269 | 0.0216 |
| LRF-225 | 1.52 | 6.25 | 90963 | 0.0119 |
| LRF-226 | 1.68 | 6.02 | 100636 | 0.0119 |
| LRF-227 | 1.42 | 5.19 | 82775 | 0.0119 |
| LRF-228 | 1.36 | 6.30 | 73388 | 0.0449 |
| LRF-230 | 1.78 | 5.13 | 121204 | 0.0119 |
| LRF-231 | 1.79 | 5.20 | 120973 | 0.0122 |
| LRF-232 | 1.56 | 4.72 | 65573 | 0.0122 |
| LRF-234 | 1.49 | 5.11 | 30589 | 0.0122 |
| LRF-235 | 8.26 | 6.90 | 88853 | 0.0075 |
| LRF-236 | 1.47 | 6.09 | 39155 | 0.0216 |
| LRF-237 | 2.08 | 5.45 | 49032 | 0.0122 |

The ninth group consists of LRFs that are decreased in the blood of subjects with full manifestation liver response as compared with the blood of subjects free from liver response. These LRFs can be described by apparent MW and pI as provided in Table IX.

Table IX. LRFs Decreased in Blood of Subjects Having Full Manifestation Liver Response

| | Relative | | | |
|---|---|---|---|---|
| | Abundance | | | P value |
| | (foreground over | | | (Rank Sum |
| LRF# | background) | pI | MW (Da) | Test) |
| LRF-256 | −1.39 | 5.06 | 38779 | 0.0119 |
| LRF-257 | −1.42 | 5.69 | 35827 | 0.0122 |
| LRF-258 | −2.15 | 5.61 | 35868 | 0.0122 |
| LRF-259 | −1.34 | 5.32 | 23373 | 0.0212 |
| LRF-260 | −5.26 | 4.97 | 46213 | 0.0075 |
| LRF-261 | −1.63 | 5.03 | 41400 | 0.0122 |
| LRF-262 | −6.83 | 4.93 | 31680 | 0.0075 |
| LRF-263 | −1.61 | 5.39 | 62144 | 0.0119 |
| LRF-264 | −1.59 | 6.78 | 53959 | 0.0122 |
| LRF-265 | −4.20 | 6.83 | 28900 | 0.0073 |
| LRF-266 | −1.98 | 5.38 | 38357 | 0.0122 |
| LRF-270 | −1.82 | 5.57 | 15056 | 0.0122 |
| LRF-271 | −1.33 | 4.67 | 51056 | 0.0122 |

The tenth group consists of LRFs that are increased in the blood of subjects with full manifestation liver response as compared with the blood of subjects free from liver response. These LRFs can be described by apparent MW and pI as provided in Table X.

Table X. LRFs Increased in Blood of Subjects Having Full Manifestation Liver Response

| | Relative | | | |
|---|---|---|---|---|
| | Abundance | | | P value |
| | (foreground over | | | (Rank Sum |
| LRF# | background) | pI | MW (Da) | Test) |
| LRF-294 | 1.57 | 4.95 | 32192 | 0.0216 |
| LRF-295 | 1.48 | 7.31 | 56943 | 0.0119 |
| LRF-296 | 1.50 | 7.21 | 28524 | 0.0117 |
| LRF-297 | 5.28 | 6.80 | 39504 | 0.0075 |
| LRF-298 | 6.40 | 7.08 | 39510 | 0.0075 |
| LRF-300 | 1.97 | 5.25 | 91060 | 0.0097 |
| LRF-301 | 12.62 | 5.67 | 81660 | 0.0075 |
| LRF-303 | 1.87 | 4.90 | 75356 | 0.0122 |
| LRF-305 | 6.03 | 6.20 | 33596 | 0.0073 |
| LRF-306 | 1.35 | 6.67 | 28449 | 0.0117 |

The eleventh group consists of LRFs that are decreased in the blood of subjects with late liver response as compared with the blood of subjects free from liver response. These LRFs can be described by apparent MW and pI as provided in Table XI.

Table XI. LRFs Decreased in Blood of Subjects Having Late Liver Response

| | Relative | | | |
|---|---|---|---|---|
| | Abundance | | | P value |
| | (foreground over | | | (Rank Sum |
| LRF# | background) | pI | MW (Da) | Test) |
| LRF-328 | −6.78 | 6.54 | 139228 | 0.0073 |
| LRF-329 | −3.14 | 5.94 | 33501 | 0.0075 |
| LRF-331 | −2.50 | 5.97 | 140431 | 0.0122 |
| LRF-332 | −2.69 | 5.85 | 61591 | 0.0122 |
| LRF-334 | −1.63 | 5.85 | 101552 | 0.0122 |
| LRF-335 | −1.89 | 6.56 | 71935 | 0.0112 |

The twelth group consists of LRFs that are increased in the blood of subjects with late liver response as compared with the blood of subjects free from liver response. These LRFs can be described by apparent MW and pi as provided in Table XII.

Table XII. LRFs Increased in Blood of Subjects Having Late Liver Response

| | Relative | | | |
|---|---|---|---|---|
| | Abundance | | | P value |
| | (foreground over | | | (Rank Sum |
| LRF# | background) | pI | MW (Da) | Test) |
| LRF-356 | 5.22 | 6.13 | 29252 | 0.0108 |
| LRF-358 | 1.22 | 5.33 | 36864 | 0.0122 |
| LRF-359 | 1.73 | 5.08 | 33850 | 0.0122 |
| LRF-360 | 2.14 | 4.86 | 36619 | 0.0122 |
| LRF-361 | 1.67 | 5.07 | 31110 | 0.0119 |
| LRF-363 | 1.62 | 5.13 | 43976 | 0.0122 |
| LRF-364 | 4.15 | 5.48 | 38709 | 0.0075 |
| LRF-365 | 1.62 | 5.71 | 72892 | 0.0119 |
| LRF-366 | 2.77 | 4.80 | 48783 | 0.0097 |
| LRF-367 | 6.89 | 4.78 | 49587 | 0.0073 |
| LRF-368 | 2.22 | 4.93 | 69794 | 0.0122 |
| LRF-369 | 2.53 | 5.25 | 19817 | 0.0097 |
| LRF-370 | 4.13 | 5.32 | 25447 | 0.0108 |
| LRF-371 | 3.02 | 4.85 | 112218 | 0.0109 |
| LRF-372 | 3.78 | 6.39 | 85568 | 0.0108 |
| LRF-373 | 1.74 | 5.63 | 12297 | 0.0122 |

Those skilled in the art will understand, based upon the present description, that a given LRF can be described according to the data provided for that LRF in any of Tables I through XII. An LRF is a protein that has a pI of about the value stated for that LRF (preferably within about 10%, more preferably within about 5% still more preferably within about 1% of the stated value) and has a MW of about the value stated for that LRF (preferably within about 10%, more preferably within about 5%, still more preferably within about 1% of the stated value).

The LRFs can be more particularly described by amino acid sequencing as noted in Tables XV through XXVI as tandem peptides. LRFs were isolated, subjected to proteolysis, and analyzed by mass spectrometry using in this instance the methods and apparatuses described herein. One skilled in the art can identify sequence information from proteins analyzed by mass spectrometry and/or tandem mass spectrometry using various spectral interpretation methods and database searching tools. Examples of some of these methods and tools can be found at the Swiss Institute of Bioinformatics web site at http://www.expasy.ch/, and the European Molecular Biology Laboratory web site at http://www.narrador.embl-heidelberg.de/GroupPages/PageLink/peptidesearchpage.html. Identification of LRPIs was performed using the SEQUEST search program (Eng et al., 1994, J. Am. Soc. Mass Spectrom. 5:976-989) as described in the Examples, infra, and the method described in PCT Application No. PCT/GB01/04034, which is incorporated herein by reference in its entirety.

For each LRF, the present invention additionally provides: (a) a preparation comprising the isolated LRF; (b) a preparation comprising one or more fragments of an LRF; and (c) antibodies that bind to said LRF, to said fragments, or both to said LRF and to said fragments. As used herein, an LRF is “isolated” when it is present in a preparation that is substantially free of other proteins, i.e., a preparation in which less than 10% (particularly less than 5%, more particularly less than 1%) of the total protein present is contaminating protein(s). Other proteins are proteins or protein isoforms having a significantly different pI or MW from those of the isolated LRF, as determined by 2D electrophoresis. As used herein, a “significantly different” pI or MW is one that permits the other protein to be resolved from the LRF on 2D electrophoresis, performed according to the Reference Protocol.

In one embodiment, an isolated protein is provided, that comprises a peptide with a determined amino acid sequence for an LRF, said protein having a pI and MW within 10% (particularly within 5%, more particularly within 1%) of the values identified in Tables I through XII for that LRF.

The LRFs of the invention can be qualitatively or quantitatively detected by any method known to those skilled in the art, including but not limited to the methods described herein, or using kinase assays, enzyme assays, binding assays and other functional assays, immunoassays, and western blotting. In one embodiment, the LRFs are separated on a 2-D gel by virtue of their MWs and pIs and are visualized by staining the gel. In one embodiment, the LRFs are stained with a fluorescent dye and imaged with a fluorescence scanner. Sypro Red (Molecular Probes, Inc., Eugene, Oreg.) is a suitable dye for this purpose. Alternative dyes are described in U.S. Ser. No. 09/412,168, filed Oct. 5, 1999, and incorporated herein by reference in its entirety.

Alternatively, LRFs can be detected in an immunoassay. In one embodiment, an immunoassay is performed by contacting a sample with an anti-LRF antibody under conditions such that immunospecific binding can occur if the LRF is present, and detecting or measuring the amount of any immunospecific binding by the antibody. Particularly, the anti-LRF antibody preferentially binds to the LRF rather than to other family members or isoforms of the same protein. In a particular embodiment, the anti-LRF antibody binds to the LRF with at least 2-fold greater affinity, more particularly at least 5-fold greater affinity, still more particularly at least 10-fold greater affinity, than to said other family members or isoforms of the same protein. One skilled in the art will understand based on the general description and their own knowledge how to generate additional antibodies by using the LRF itself for the generation of such antibodies.

For any given LRF, the signal obtained upon analyzing blood or liver from subjects having a liver response relative to the signal obtained upon analyzing blood or liver from subjects free from liver response will depend upon the particular analytical protocol and detection technique that is used. Accordingly, those skilled in the art will understand that any laboratory, based on the present description, can establish a suitable reference range for any LRF in subjects free from liver response according to the analytical protocol and detection technique in use. Preferably, at least one control positive blood or liver sample from a subject known to have liver response or at least one control negative blood or liver sample from a subject known to be free from liver response (and more preferably both positive and negative control samples) are included in each batch of test samples analyzed. In one embodiment, the level of expression of a feature is determined relative to a background value, which is defined as the level of signal obtained from a proximal region of the image that (a) is equivalent in area to the particular feature in question, and (b) contains no substantially discernable protein feature.

In a preferred embodiment, the signal associated with an LRF in the blood or liver of a subject (e.g., a subject suspected of having or known to have liver response) is normalized with reference to one or more ERFs detected in the same 2D gel. As will be apparent to one of ordinary skill in the art, such ERFs may readily be determined by comparing different samples using any of a variety of techniques.

As those of skill in the art will readily appreciate, the measured MW and pI of a given feature or protein isoform will vary to some extent depending on the precise protocol used for each step of the 2D electrophoresis and for landmark matching. As used herein, the terms “MW” and “pI” are defined, respectively, to mean the apparent molecular weight and the apparent isoelectric point of a feature or protein isoformn as measured in careful accordance with the Reference Protocol identified in Section 6 below. When the Reference Protocol is followed and when samples are run in duplicate or a higher number of replicates, variation in the measured mean pI of an LRF or LRPI is generally less than 3% and variation in the measured mean MW of an LRF or LRPI is generally less than 5%. Where the skilled artisan wishes to diverge from the Reference Protocol, calibration experiments should be performed to compare the MW and pI for each LRF or protein isoform as detected (a) by the Reference Protocol and (b) by the divergent protocol.

The LRFs of the invention can be used, for example, for detection, prognosis, diagnosis, or monitoring of liver response or for the development of pharmaceutical products. In one embodiment of the invention, liver tissue from a subject (e.g., a subject suspected of having liver response) is analyzed by 2D electrophoresis for quantitative detection of one or more of the LRFs as defined in anyone of Tables I, III or V in any suitable combination. A decreased abundance of one or more of such LRFs in the liver tissue from the subject relative to liver tissue from a subject or subjects free from liver response indicates the presence of a liver response.

In another embodiment of the invention, liver tissue from a subject is analyzed by 2D electrophoresis for quantitative detection of one or more of the LRFs as defined in anyone of Tables II, IV or VI, either individually or in any suitable combination. An increased abundance of one or more, or any suitable combination, of LRFs in the liver tissue from the subject relative to liver tissue from a subject or subjects free from liver response (e.g., a control sample or a previously determined reference range) indicates the presence of liver response.

In yet another embodiment, liver tissue from a subject is analyzed by 2D electrophoresis for quantitative detection of (a) one or more LRFs or any suitable combination, whose decreased abundance indicates the presence of liver response, e.g. the LRFs as defined in any one of Tables I, III or V, and (b) one or more LRFs or any suitable combination, whose increased abundance indicates the presence of a liver response e.g. the LRFs as defined in anyone of Tables II, IV or VI.

In yet another embodiment of the invention, blood from a subject is analyzed by 2D electrophoresis for quantitative detection of one or more of the LRFs as defined in any one of Tables VII, IX or XI. A decreased abundance of one or more LRFs in the blood from the subject relative to blood from a subject or subjects free from liver response (e.g., a control sample or a previously determined reference range) indicates the presence of liver response.

In yet a further embodiment of the invention, blood from a subject is analyzed by 2D electrophoresis for quantitative detection of one or more of the LRFs as defined in Tables VIII, X or XII. An increased abundance of one or more LRFs in the blood sample from the subject, relative to blood sample(s) from one or more subjects free from liver response (e.g., (a) control sample(s) or a previously determined reference range) indicates the presence of liver response.

In yet another embodiment, blood from a subject is analyzed by 2D electrophoresis for quantitative detection of (a) one or more LRFs or any suitable combination of them, whose decreased abundance indicates the presence of liver response, e.g. the LRFs as defined in any one of Tables VII, IX or IX,; and (b) one or more LRFs or any suitable combination of them, whose increased abundance indicates the presence of a liver response, e.g. the LRF as defined in any one of Tables XIII, X or XII.

In yet another embodiment of the invention, liver tissue from a subject is analyzed by 2D electrophoresis for quantitative detection of one or more of the LRFs as defined in Tables I to VI, wherein the ratio of the one or more LRFs relative to an Expression Reference Feature (ERF) indicates that liver response is present. In a specific embodiment, a decrease in one or more LRF/ERF ratios in a sample being tested relative to the LRF/ERF ratios in a control sample or a reference range indicates the presence of liver response; the LRFs as defined in any one of Tables I, III or V are suitable LRFs for this purpose. In another specific embodiment, an increase in one or more LRF/ERF ratios in a test sample relative to the LRF/ERF ratios in a control sample or a reference range indicates the presence of liver response; the LRFs as defined in any one of Tables II, IV or VI are suitable LRFs for this purpose.

In a further embodiment of the invention, liver tissue from a subject is analyzed by 2D electrophoresis for quantitative detection of (a) one or more LRFs, or any suitable combination, whose decreased LRF/ERF ratio(s) in a test sample relative to the LRF/ERF ratio(s) in a control sample indicates the presence of liver response, e.g. the LRFs as defined in any one of Tables I, III or V; and (b) one or more LRFs, or any suitable combination, whose increased LRF/ERF ratio(s) in a test sample relative to the LRF/ERF ratio(s) in a control sample indicates the presence of liver response, e.g. the LRFs as defined in any one of Tables II, IV or VI.

In yet another embodiment of the invention, blood from a subject is analyzed by 2D electrophoresis for quantitative detection of one or more of the LRFs as defined in any one of Tables VII to XII, wherein the ratio of the one or more LRFs relative to an Expression Reference Feature (ERF) indicates whether liver response is present. In a specific embodiment, a decrease in one or more LRF/ERF ratios in a test sample relative to the LRF/ERF ratios in a control sample or a reference range indicates the presence of liver response; the LRFs as defined in any one of Tables VII, IX or XI are suitable LRFs for this purpose. In another specific embodiment, an increase in one or more LRF/ERF ratios in a test sample relative to the LRF/ERF ratios in a control sample or a reference range indicates the presence of liver response; the LRFs as defined in any one of Tables VIII, X or XII are suitable LRFs for this purpose.

In a further embodiment of the invention, blood from a subject is analyzed by 2D electrophoresis for quantitative detection of (a) one or more LRFs, or any suitable combination, whose decreased LRF/ERF ratio(s) in a test sample relative to the LRF/ERF ratio(s) in a control sample indicates the presence of liver response, e.g. the LRFs as defined in any one of Tables VII, IX or XI; and (b) one or more LRFs, or any suitable combination, whose increased LRF/ERF ratio(s) in a test sample relative to the LRF/ERF ratio(s) in a control sample indicates the presence of liver response, e.g. the LRFs as defined in any one of Tables VIII, X or XII.

In a preferred embodiment, blood or liver is analyzed for quantitative detection of a plurality of LRFs.

Liver Response-Associated Protein Isoforms (LRPIs)

In another aspect of the invention, blood or liver is analyzed for quantitative detection of one or more Liver Response-Associated Protein Isoforms (LRPIs), e.g. for screening or diagnosis of a liver response, to determine the prognosis of a subject having liver response, to monitor the effectiveness of liver response therapy, to identify patients who may have an adverse response to a particular therapeutic treatment or for development of pharmaceutical products. As is well known in the art, a given protein may be expressed as one or more variant forms (isoforms) that differ in amino acid composition (e.g., as a result of alternative mRNA or pre-mRNA processing e.g., as a result of alternative splicing or limited proteolysis), as a result of differential post-translational modification (e.g., glycosylation, phosphorylation, acylation), or both, so that proteins of identical amino acid sequence can differ in their pI, MW, or both. “Liver Response-Associated Protein Isoform” refers to a protein isoform present in blood or liver from a subject having liver response.

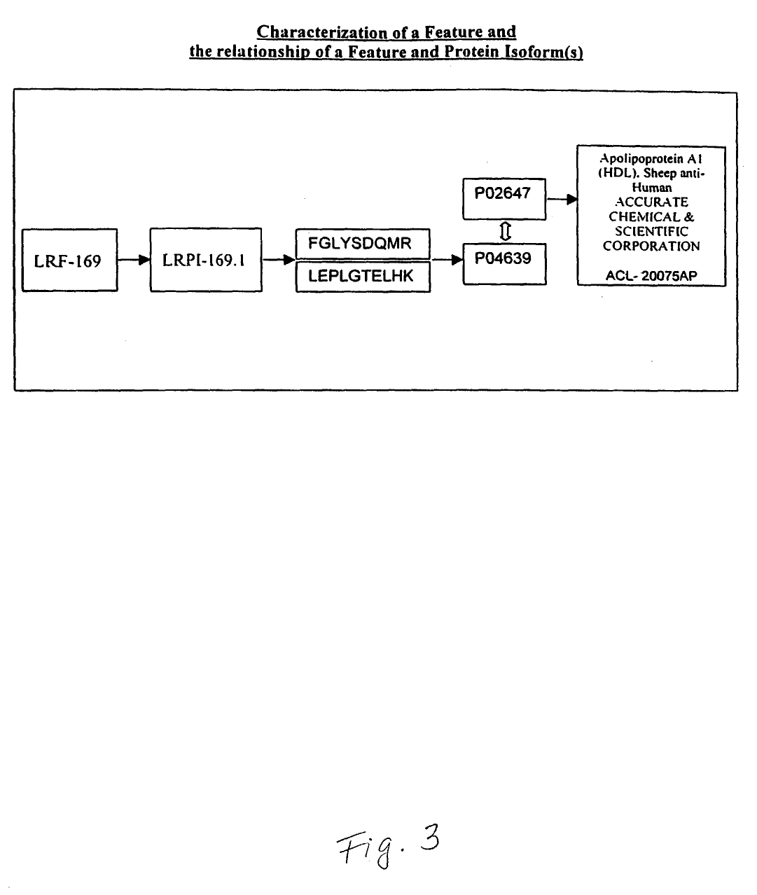

Twelve groups of LRPIs are described herein by the amino acid sequencing of LRFs, as depicted in FIG. 3 and as described above. LRPIs were isolated, subjected to proteolysis, and analyzed by mass spectrometry as described herein. One skilled in the art can identify sequence information from proteins analyzed by mass spectrometry and/or tandem mass spectrometry using various spectral interpretation methods and database searching tools. Examples of some of these methods and tools can be found at the Swiss Institute of Bioinformatics web site at http://www.expasy.ch/, and the European Molecular Biology Laboratory web site at www.narrador.embl-heidelberg.de/GroupPages/PageLink/peptidesearchpage.html. Identification of AAPIs was performed using the SEQUEST search program (Eng et al., 1994, J. Am. Soc. Mass Spectrom. 5:976-989) and the method described in WO 02/21139, which is incorporated herein by reference in its entirety.

The first group comprises LRPIs that are significantly decreased in the liver tissue of subjects with early liver response as compared with the liver tissue of subjects free from liver response. The amino acid sequences of peptides produced from these LRPIs by proteolysis using trypsin and identified by tandem mass spectrometry, and database searching using the SEQUEST program are listed in Table XIII.

Table XIII. LRPIs Decreased in Liver Tissue of Subjects Having Early Liver Response

TABLE XIII

| | | | MW | Amino Acid Sequence of | |
|---|---|---|---|---|---|
| LRF# | LRPI# | pI | (Da) | Tryptic Digest Peptides | SEQ ID NO: |
| LRF-1 | LRPI-1.1 | 8.95 | 16081 | MNLGVGAYR | SEQ ID NO: 265 |
| | | | | DDNGKPYVLPSVR | SEQ ID NO: 56 |
| | | | | DAGMQLQGYR | SEQ ID NO: 54 |
| | | | | FVTVQTISGTGALR | SEQ ID NO. 150 |
| LRF-2 | LRPI-2.1 | 4.60 | 62363 | IAELFSDLEER | SEQ ID NO: 188 |
| LRF-3 | LRPI-3.1 | 6.59 | 12894 | VTLTPDEEAR | SEQ ID NO: 390 |
| LRF-4 | LRPI-4.1 | 4.93 | 26181 | DTYLSHFNPR | SEQ ID NO: 79 |
| LRF-386 | LRPI-386.1 | 5.01 | 27830 | SLLVTELGSSR | SEQ ID NO: 321 |
| | | | | EIEIIPLQR | SEQ ID NO: 98 |
| LRF-387 | LRPI-387.1 | 5.44 | 24451 | WNEEVEAYR | SEQ ID NO: 398 |
| | | | | LEPLGTELHK | SEQ ID NO: 229 |

The second group comprises LRPIs that are increased in the liver tissue of subjects with early liver response as compared with the liver tissue of subjects free from liver response. The amino acid sequences of tryptic digest peptides of these LRPIs identified by tandem mass spectrometry and database searching using the SEQUEST program are listed in Table XIV.

Table XIV. LRPIs Increased in Liver Tissue of Subjects Having Early Liver Response

| | | | | Amino Acid Sequences of | |
|---|---|---|---|---|---|
| LRF# | LRPI# | pI | MW (Da) | Tryptic Digest Peptides | SEQ ID NO: |
| LRF-6 | LRPI-6.1 | 8.36 | 31027 | DTPGFIVNR | SEQ ID NO: 78 |
| LRF-8 | LRPI-8.1 | 5.57 | 23697 | WNEEVEAYR | SEQ ID NO: 398 |
| | | | | LEPLGTELHK | SEQ ID NO: 229 |
| | | | | VKDFATVYVDAVK | SEQ ID NO: 369 |
| | | | | MQPHLDEFQEK | SEQ ID NO: 268 |
| | | | | FGLYSDQMR | SEQ ID NO: 135 |
| LRF-9 | LRPI-9.1 | 4.79 | 37664 | LLFEGAGSNPGDK | SEQ ID NO: 240 |
| LRF-10 | LRPI-10.1 | 7.73 | 123358 | GVPTGFVLPIR | SEQ ID NO: 176 |
| | | | | TPVPSDIAISR | SEQ ID NO: 349 |
| LRF-11 | LRPI-11.1 | 6.25 | 79681 | GQNQPVLNITNR | SEQ ID NO: 167 |
| LRF-11 | LRPI-11.2 | 6.25 | 79681 | QLQALSSELAQAR | SEQ ID NO: 298 |
| | | | | APDFVFYAPR | SEQ ID NO: 26 |
| | | | | ALELEQER | SEQ ID NO: 22 |
| | | | | FVIKPIDK | SEQ ID NO: 149 |
| LRF-11 | LRPI-11.3 | 6.25 | 79681 | FMAPDIEAAHR | SEQ ID NO: 141 |
| LRF-12 | LRPI-12.1 | 7.67 | 61649 | LAQEDPDYGLR | SEQ ID NO: 223 |
| LRF-13 | LRPI-13.1 | 5.78 | 11661 | FLTEELSLDQDR | SEQ ID NO: 139 |
| | | | | LCAATATILDKPEDR | SEQ ID NO: 224 |
| | | | | FFPLEPWQIGK | SEQ ID NO: 132 |
| LRF-14 | LRPI-14.1 | 4.99 | 56166 | VELQELNDR | SEQ ID NO: 363 |
| | | | | FADLSEAANR | SEQ ID NO: 125 |
| LRF-15 | LRPI-15.1 | 9.38 | 80495 | NQPMTPEAVR | SEQ ID NO: 285 |
| | | | | AAVAVPSRPPDAVLR | SEQ ID NO: 2 |
| | | | | HVLQQFADNDVSR | SEQ ID NO: 186 |
| | | | | VVLVTGAGGGLGR | SEQ ID NO: 395 |
| | | | | ISDEDWDIIQR | SEQ ID NO: 208 |
| LRF-16 | LRPI-16.1 | 5.81 | 30167 | LIEFLER | SEQ ID NO: 237 |
| | | | | NHFPEALR | SEQ ID NO: 277 |
| | | | | IPYLECR | SEQ ID NO: 206 |
| LRF-17 | LRPI-17.1 | 5.81 | 30167 | CKEDAIFNR | SEQ ID NO: 47 |
| | | | | IIQHTSFQEMK | SEQ ID NO: 196 |
| | | | | LIEFLER | SEQ ID NO: 237 |
| | | | | NHFPEALR | SEQ ID NO: 277 |
| | | | | IPYLECR | SEQ ID NO: 206 |
| LRF-17 | LRPI-17.2 | 5.81 | 30167 | IIQHTSFQEMK | SEQ ID NO: 196 |
| | | | | LIEFLER | SEQ ID NO: 237 |
| | | | | IPDLECR | SEQ ID NO: 203 |
| | | | | CKEDALFNR | SEQ ID NO: 48 |
| | | | | NHFPEALR | SEQ ID NO: 277 |
| LRF-18 | LRPI-18.1 | 5.46 | 40000 | FPPDNSAPYGAR | SEQ ID NO: 144 |
| | | | | SRPSLPLPQSR | SEQ ID NO: 325 |
| | | | | DFDPAINEYIQR | SEQ ID NO: 61 |
| LRF-19 | LRPI-19.1 | 5.83 | 22631 | FLVGPDGVPVR | SEQ ID NO: 140 |
| LRF-20 | LRPI-20.1 | 6.35 | 28978 | GSAPPGPVPEGQIR | SEQ ID NO: 169 |
| LRF-21 | LRPI-21.1 | 8.25 | 35532 | GAAQNIIPASTGAAK | SEQ ID NO: 152 |
| | | | | VPTPNVSVVDLTCR | SEQ ID NO: 382 |
| | | | | PITIFQER | SEQ ID NO: 291 |
| | | | | LISWYDNEYGYSNR | SEQ ID NO: 238 |
| LRF-22 | LRPI-22.1 | 9.80 | 11450 | FGDLSSASAIMGNPQVK | SEQ ID NO: 133 |
| | | | | GTFAHLSELHCDK | SEQ ID NO: 173 |
| | | | | LLVVYPWTQR | SEQ ID NO: 244 |
| | | | | ATVSGLWGK | SEQ ID NO: 38 |
| | | | | EFTPSAQAAFQK | SEQ ID NO: 93 |
| LRF-23 | LRPI-23.1 | 8.06 | 31812 | NVNCSIEESFQR | SEQ ID NO: 288 |
| | | | | EVSIFGAASELFTR | SEQ ID NO: 122 |
| | | | | FDGVMQAAR | SEQ ID NO: 127 |
| | | | | LLEAGDFICQALNR | SEQ ID NO: 239 |
| LRF-24 | LRPI-24.1 | 10.76 | 23549 | VLDELTLTK | SEQ ID NO: 370 |
| | | | | QSLEASLAETEGR | SEQ ID NO: 300 |
| LRF-25 | LRPI-25.1 | 4.80 | 26633 | ALEQANGELEVK | SEQ ID NO: 23 |
| LRF-26 | LRPI-26.1 | 5.28 | 58041 | AAVEEGIVLGGGCALLR | SEQ ID NO: 3 |
| | | | | TLNDELEIIEGMK | SEQ ID NO: 348 |
| | | | | CIPALDSLKPANEDQK | SEQ ID NO: 46 |
| | | | | VTDALNATR | SEQ ID NO: 387 |
| LRF-27 | LRPI-27.1 | 7.31 | 124197 | QINLSNIR | SEQ ID NO: 295 |
| LRF-28 | LRPI-28.1 | 5.65 | 49698 | QTWGTVFR | SEQ ID NO: 303 |
| | | | | LRPVAGLLSSR | SEQ ID NO: 251 |
| | | | | QFADIAYNYR | SEQ ID NO: 293 |
| LRF-29 | LRPI-29.1 | 6.19 | 77203 | VPVIRPDVAK | SEQ ID NO: 383 |
| | | | | VVEEAPSIFLDPETR | SEQ ID NO: 392 |
| LRF-30 | LRPI-30.1 | 4.73 | 57645 | VDATEESDLAQQYGVR | SEQ ID NO: 362 |
| LRF-31 | LRPI-31.1 | 8.97 | 27694 | CLPLSMDVR | SEQ ID NO: 49 |
| | | | | VNSLAPGAISGTEGLR | SEQ ID NO: 376 |
| | | | | GQVLQLHAGAAK | SEQ ID NO: 168 |
| LRF-32 | LRPI-32.1 | 5.63 | 77203 | AEAEAQAEELSFPR | SEQ ID NO: 8 |
| LRF-36 | LRPI-36.1 | 5.73 | 74823 | APQVSTPTLVEAAR | SEQ ID NO: 28 |
| LRF-37 | LRPI-37.1 | 7.59 | 33242 | TVSVLNGGFR | SEQ ID NO: 354 |
| LRF-38 | LRPI-38.1 | 5.02 | 56260 | VELQELNDR | SEQ ID NO: 363 |
| | | | | SYVTTSTR | SEQ ID NO: 333 |
| | | | | LQEEMLQR | SEQ ID NO: 249 |
| | | | | LGDLYEEEMR | SEQ ID NO: 232 |
| | | | | ILLAELEQLK | SEQ ID NO: 197 |
| LRF-389 | LRPI-389.1 | 9.76 | 18844 | DMTLGSVLGR | SEQ ID NO: 69 |
| | | | | EVAWNLTSVDLVR | SEQ ID NO: 119 |
| LRF-390 | LRPI-390.1 | 6.36 | 46232 | GLVPEQPVTLR | SEQ ID NO: 163 |
| | | | | DETIPPVTILR | SEQ ID NO: 59 |
| | | | | HFMAPGVR | SEQ ID NO: 182 |
| | | | | SCWDEPLSITVR | SEQ ID NO: 311 |
| | | | | ADAHGELDLAR | SEQ ID NO: 6 |
| LRF-391 | LRPI-391.1 | 6.97 | 28172 | GSAPPGPVPEGQIR | SEQ ID NO: 169 |
| LRF-392 | LRPI-392.1 | 7.98 | 29700 | DETNYGIPQR | SEQ ID NO: 60 |
| | | | | LWDLTTGTTTR | SEQ ID NO: 256 |
| | | | | DVLSVAFSSDNR | SEQ ID NO: 81 |
| LRF-393 | LRPI-393.1 | 8.88 | 28876 | IQDFQEDEELFR | SEQ ID NO: 207 |
| | | | | NLVSDDDIQR | SEQ ID NO: 281 |
| | | | | NNGCLVVLPGTHK | SEQ ID NO: 283 |
| LRF-394 | LRPI-394.1 | 6.50 | 25990 | QTESTSFLEK | SEQ ID NO: 302 |
| | | | | AINQGGLTSVAVR | SEQ ID NO: 18 |
| LRF-394 | LRPI-394.2 | 6.50 | 25990 | SLLEGEGSSGGGGR | SEQ ID NO: 320 |
| LRF-395 | LRPI-395.1 | 9.74 | 25549 | SHGQDYLVGNR | SEQ ID NO: 315 |
| | | | | SLHVYLEK | SEQ ID NO: 319 |
| LRF-397 | LRPI-397.1 | 5.74 | 68828 | CCTLPEAQR | SEQ ID NO: 43 |
| | | | | APQVSTPTLVEAAR | SEQ ID NO: 28 |
| LRF-399 | LRPI-399.1 | 5.83 | 67405 | CCTLPEAQR | SEQ ID NO: 43 |
| | | | | APQVSTPTLVEAAR | SEQ ID NO: 28 |
| | | | | LPCVEDYLSAILNR | SEQ ID NO: 246 |
| | | | | ECCHGDLLECADDR | SEQ ID NO: 84 |
| | | | | SIHTLFGDK | SEQ ID NO: 317 |

The third group consists of LRPIs that are decreased in the liver tissue of subjects with full manifestation liver response as compared with the liver tissue of subjects free from liver response. The amino acid sequences of tryptic digest peptides of these LRPIs identified by tandem mass spectrometry and database searching using the SEQUEST program are listed in Table XV.

Table XV. LRPIs Decreased in Liver Tissue of Subjects Having Full Manifestation Liver Response

| | | | MW | Amino Acid Sequences of | |
|---|---|---|---|---|---|
| LRF# | LRPI# | pI | (Da) | Tryptic Digest Peptides | SEQ ID NO: |
| LRF-53 | LRPI-53.1 | 6.14 | 72138 | QGEIFLLPAR | SEQ ID NO: 294 |
| | | | | EPPFPLSTR | SEQ ID NO: 114 |
| LRF-54 | LRPI-54.1 | 4.81 | 51921 | FTQAGSEVSALLGR | SEQ ID NO: 147 |
| | | | | VALVYGQMNEPPGAR | SEQ ID NO: 359 |
| | | | | IPVGPETLGR | SEQ ID NO: 205 |
| | | | | VALTGLTVAEYFR | SEQ ID NO: 358 |
| | | | | IMNVIGEPIDER | SEQ ID NO: 200 |
| LRF-56 | LRPI-56.1 | 6.05 | 45895 | GIYMWDVEGR | SEQ ID NO: 161 |
| LRF-56 | LRPI-56.2 | 6.05 | 45895 | LPSDVVTAVR | SEQ ID NO: 247 |
| LRF-386 | LRPI-386.1 | 5.01 | 27830 | SLLVTELGSSR | SEQ ID NO: 321 |
| | | | | EIEIIPLQR | SEQ ID NO: 98 |
| LRF-387 | LRPI-387.1 | 5.44 | 24451 | WNEEVEAYR | SEQ ID NO: 398 |
| | | | | LEPLGTELHK | SEQ ID NO: 229 |
| LRF-400 | LRPI-400.1 | 8.84 | 11701 | VNPDDVGGEALGR | SEQ ID NO: 374 |
| | | | | LLVVYPWTQR | SEQ ID NO: 244 |
| LRF-400 | LRPI-400.2 | 8.84 | 11701 | STGGAPTFNVTVTMTAK | SEQ ID NO: 329 |
| | | | | EGVHGGLINK | SEQ ID NO: 94 |
| | | | | SSFFVNGLTLGGQK | SEQ ID NO: 326 |
| | | | | DSLLQDGEFTMDLR | SEQ ID NO: 75 |
| | | | | DSPSVWAAVPGK | SEQ ID NO: 76 |
| | | | | CYEMASHLR | SEQ ID NO: 51 |

The fourth group consists of LRPIs that are increased in the liver tissue of subjects with full manifestation liver response as compared with the liver tissue of subjects free from liver response. The amino acid sequences of tryptic digest peptides of these LRPIs identified by tandem mass spectrometry and database searching using the SEQUEST program are listed in Table XVI.

Table XVI. LRPIs Increased in Liver Tissue of Subjects Having Full Manifestation Liver Response

| | | | | Amino Acid Sequences of | |
|---|---|---|---|---|---|
| LRF# | LRPI# | pI | MW (Da) | Tryptic Digest Peptides | SEQ ID NO: |
| LRF-60 | LRPI-60.1 | 6.80 | 37664 | TQAQIVLR | SEQ ID NO: 350 |
| | | | | SNLCATWEALEACK | SEQ ID NO: 324 |
| | | | | ENFQIFDFSLTK | SEQ ID NO: 108 |
| LRF-61 | LRPI-61.1 | 8.08 | 14573 | LVGGMVSYLNDLPSQR | SEQ ID NO: 255 |
| | | | | AFTTWTANAGIEECR | SEQ ID NO: 11 |
| LRF-63 | LRPI-63.1 | 6.02 | 46636 | TVIFTQGR | SEQ ID NO: 351 |
| | | | | VAQWMIQEPHR | SEQ ID NO: 360 |
| | | | | YSLKPNDQILAEDK | SEQ ID NO: 407 |
| LRF-64 | LRPI-64.1 | 5.87 | 58635 | EDFPFLR | SEQ ID NO: 86 |
| LRF-66 | LRPI-66.1 | 5.44 | 24451 | WNEEVEAYR | SEQ ID NO: 398 |
| LRF-67 | LRPI-67.1 | 4.90 | 33525 | TANLGAGAAQPLR | SEQ ID NO: 337 |
| | | | | GWFEPLVEDMQR | SEQ ID NO: 178 |
| | | | | LGPLVEQGR | SEQ ID NO: 234 |
| | | | | NEVNTMLGQSTEELR | SEQ ID NO: 273 |
| LRF-69 | LRPI-69.1 | 7.67 | 40789 | TDDSQPWVLPVVR | SEQ ID NO: 338 |
| | | | | IVATTLSNPELFK | SEQ ID NO: 213 |
| | | | | NFGLYNER | SEQ ID NO: 275 |
| | | | | SCASQLVLGDNSPALR | SEQ ID NO: 310 |
| | | | | VGGVQSLGGTGALR | SEQ ID NO: 367 |
| LRF-70 | LRPI-70.1 | 9.50 | 11592 | VNPDNVGAEALGR | SEQ ID NO: 375 |
| LRF-71 | LRPI-71.1 | 7.80 | 27225 | GGPLSGPYR | SEQ ID NO: 160 |
| | | | | VVFDDTFDR | SEQ ID NO: 394 |
| LRF-72 | LRPI-72.1 | 4.69 | 54509 | GFGHLLQR | SEQ ID NO: 157 |
| LRF-73 | LRPI-73.1 | 7.82 | 55445 | FEEEGNPYYSSAR | SEQ ID NO: 129 |
| LRF-74 | LRPI-74.1 | 6.70 | 92196 | ISDIPVTAIR | SEQ ID NO: 209 |
| LRF-75 | LRPI-75.1 | 7.58 | 30261 | APSSSSAGISEWLDQK | SEQ ID NO: 29 |
| LRF-75 | LRPI-75.2 | 7.58 | 30261 | VPMLEQNVPGAR | SEQ ID NO: 381 |
| | | | | TLDEHTLER | SEQ ID NO: 346 |
| LRF-76 | LRPI-76.1 | 5.24 | 52175 | YEALLLTHESSIR | SEQ ID NO: 403 |
| LRF-77 | LRPI-77.1 | 7.04 | 22103 | KPPPDGHYVDVVR | SEQ ID NO: 218 |
| | | | | LYYFQGR | SEQ ID NO: 257 |
| | | | | EESLALAVK | SEQ ID NO: 90 |
| LRF-78 | LRPI-78.1 | 6.36 | 32515 | LTEIINTQHENVK | SEQ ID NO: 253 |
| | | | | ELHSILQHK | SEQ ID NO: 102 |
| | | | | VTMWVFEEDIGGR | SEQ ID NO: 391 |
| LRF-79 | LRPI-79.1 | 7.52 | 24017 | NVPNWHR | SEQ ID NO: 289 |
| | | | | HLTGEFEK | SEQ ID NO: 184 |
| | | | | NLQYYDISAK | SEQ ID NO: 280 |
| LRF-79 | LRPI-79.2 | 7.52 | 24017 | EIDQEAAVEVSQLR | SEQ ID NO: 97 |
| | | | | DPQHDLER | SEQ ID NO: 71 |
| | | | | VPDFSDYR | SEQ ID NO: 379 |
| LRF-80 | LRPI-80.1 | 5.12 | 127612 | EFSITDVVPYPISLR | SEQ ID NO: 92 |
| | | | | QDLPALEEKPR | SEQ ID NO: 292 |
| | | | | EDIYAVEIVGGATR | SEQ ID NO: 87 |
| | | | | FLEMCDDLLAR | SEQ ID NO: 138 |
| | | | | AGGIETIANEYSDR | SEQ ID NO: 15 |
| LRF-81 | LRPI-81.1 | 6.61 | 41576 | ILLTEPPMNPTK | SEQ ID NO: 198 |
| | | | | GYAFNHSADFETVR | SEQ ID NO: 179 |
| LRF-83 | LRPI-83.1 | 5.00 | 15334 | INPDGSQSVVEVPYAR | SEQ ID NO: 202 |
| | | | | EYGEQIDPSTHR | SEQ ID NO: 123 |
| LRF-84 | LRPI-84.1 | 9.26 | 52497 | NMQDMVEDYR | SEQ ID NO: 282 |
| LRF-85 | LRPI-85.1 | 6.29 | 128480 | QLLCDLVGISR | SEQ ID NO: 297 |
| | | | | YAEIYGISSAHTLLR | SEQ ID NO: 401 |
| | | | | LSYGPEEK | SEQ ID NO: 252 |
| | | | | SSVVPVEGCPELPHK | SEQ ID NO: 328 |
| | | | | EIYGPILER | SEQ ID NO: 101 |
| LRF-87 | LRPI-87.1 | 5.58 | 28251 | ISELDAFLK | SEQ ID NO: 210 |
| | | | | APLDIPVPDPVK | SEQ ID NO: 27 |
| | | | | IEDGNNFGVAVQEK | SEQ ID NO: 191 |
| LRF-88 | LRPI-88.1 | 6.21 | 23277 | FGPYYTEPVIAGLDPK | SEQ ID NO: 136 |
| LRF-89 | LRPI-89.1 | 6.32 | 40144 | TLEVEIEPGVR | SEQ ID NO: 347 |
| | | | | LALQLHPDR | SEQ ID NO: 222 |
| | | | | GEGLPNFDNNNIK | SEQ ID NO: 155 |
| | | | | FQDLGAAYEVLSDSEK | SEQ ID NO: 145 |
| LRF-90 | LRPI-90.1 | 5.14 | 106705 | MTNGFSGADLTEICQR | SEQ ID NO: 269 |
| | | | | MDELQLFR | SEQ ID NO: 258 |
| | | | | GGNIGDGGGAADR | SEQ ID NO: 159 |
| LRF-91 | LRPI-91.1 | 6.21 | 39258 | GPSFPEPLR | SEQ ID NO: 165 |
| LRF-92 | LRPI-92.1 | 6.51 | 59855 | AYICAHPLDR | SEQ ID NO: 42 |
| LRF-389 | LRPI-389.1 | 9.76 | 18844 | DMTLGSVLGR | SEQ ID NO: 69 |
| | | | | EVAWNLTSVDLVR | SEQ ID NO: 119 |
| LRF-390 | LRPI-390.1 | 6.36 | 46232 | GLVPEQPVTLR | SEQ ID NO: 163 |
| | | | | DETIPPVTILR | SEQ ID NO: 59 |
| | | | | HFMAPGVR | SEQ ID NO: 182 |
| | | | | SCWDEPLSITVR | SEQ ID NO: 311 |
| | | | | ADAHGELDLAR | SEQ ID NO: 6 |
| LRF-392 | LRPI-392.1 | 7.98 | 29700 | DETNYGIPQR | SEQ ID NO: 60 |
| | | | | LWDLTTGTTTR | SEQ ID NO: 256 |
| | | | | DVLSVAFSSDNR | SEQ ID NO: 81 |
| LRF-393 | LRPI-393.1 | 8.88 | 28876 | IQDFQEDEELFR | SEQ ID NO: 207 |
| | | | | NLVSDDDIQR | SEQ ID NO: 281 |
| | | | | NNGCLVVLPGTHK | SEQ ID NO: 283 |
| LRF-394 | LRPI-394.1 | 6.50 | 25990 | QTESTSFLEK | SEQ ID NO: 302 |
| | | | | AINQGGLTSVAVR | SEQ ID NO: 18 |
| LRF-394 | LRPI-394.2 | 6.50 | 25990 | SLLEGEGSSGGGGR | SEQ ID NO: 320 |
| LRF-395 | LRPI-395.1 | 9.74 | 25549 | SHGQDYLVGNR | SEQ ID NO: 315 |
| | | | | SLHVYLEK | SEQ ID NO: 319 |
| LRF-397 | LRPI-397.1 | 5.74 | 68828 | CCTLPEAQR | SEQ ID NO: 43 |
| | | | | APQVSTPTLVEAAR | SEQ ID NO: 28 |
| LRF-399 | LRPI-399.1 | 5.83 | 67405 | CCTLPEAQR | SEQ ID NO: 43 |
| | | | | APQVSTPTLVEAAR | SEQ ID NO: 28 |
| | | | | LPCVEDYLSAILNR | SEQ ID NO: 246 |
| | | | | ECCHGDLLECADDR | SEQ ID NO: 84 |
| | | | | SIHTLFGDK | SEQ ID NO: 317 |
| LRF-401 | LRPI-401.1 | 7.13 | 39509 | ETEYNVR | SEQ ID NO: 117 |
| | | | | VMEETFSYLLGR | SEQ ID NO: 373 |
| | | | | DVDPGEHYIIK | SEQ ID NO: 80 |
| LRF-402 | LRPI-402.1 | 7.37 | 51294 | HGGTIPVVPTAEFQDR | SEQ ID NO: 183 |
| | | | | TFVVQGFGNVGLHSMR | SEQ ID NO: 343 |
| | | | | DIVHSGLAYTMER | SEQ ID NO: 67 |
| | | | | MVEGFFDR | SEQ ID NO: 270 |
| LRF-403 | LRPI-403.1 | 7.83 | 51921 | VLSIGDGIAR | SEQ ID NO: 372 |
| | | | | HALIIYDDLSK | SEQ ID NO: 181 |
| LRF-405 | LRPI-405.1 | 6.76 | 30546 | SYPGSQLDILIDQGK | SEQ ID NO: 332 |
| LRF-405 | LRPI-405.2 | 6.76 | 30546 | SLVNELTFTA | SEQ ID NO: 323 |
| | | | | ELVECFQK | SEQ ID NO: 105 |
| LRF-407 | LRPI-407.1 | 6.04 | 25363 | TVMGDFAQFVDK | SEQ ID NO: 353 |
| LRF-409 | LRPI-409.1 | 5.59 | 27762 | ISELDAFLK | SEQ ID NO: 210 |
| | | | | APLDIPVPDPVK | SEQ ID NO: 27 |
| | | | | LMVMEIR | SEQ ID NO: 245 |
| | | | | TENLLGSYFPK | SEQ ID NO: 340 |
| | | | | IEDGNNFGVAVQEK | SEQ ID NO: 191 |
| | | | | EPALNEANLSNLK | SEQ ID NO: 111 |
| LRF-410 | LRPI-410.1 | 10.23 | 38026 | GGGGNFGPGPGSNFR | SEQ ID NO: 158 |
| | | | | GFGFVTFDDHDPVDK | SEQ ID NO: 156 |
| | | | | IDTIEIITDR | SEQ ID NO: 190 |

The fifth group consists of LRPIs that are decreased in the liver tissue of subjects with late liver response as compared with the liver tissue of subjects free from liver response. The amino acid sequences of tryptic digest peptides of these LRPIs identified by tandem mass spectrometry and database searching using the SEQUEST program are listed in Table XVII.

Table XVII. LRPIs Decreased in Liver Tissue of Subjects Having Late Liver Response

TABLE XVII

| | | | | Amino Acid Sequences of | |
|---|---|---|---|---|---|
| LRF# | LRPI# | pI | MW (Da) | Tryptic Digest Peptides | SEQ ID NO: |
| LRF-107 | LRPI-107.1 | 4.66 | 13804 | TYIIGELHPDDR | SEQ ID NO: 357 |
| | | | | FLEEHPGGEEVLR | SEQ ID NO: 137 |
| LRF-400 | LRPI-400.1 | 8.84 | 11701 | VNPDDVGGEALGR | SEQ ID NO: 374 |
| | | | | LLVVYPWTQR | SEQ ID NO: 244 |
| LRF-400 | LRPI-400.2 | 8.84 | 11701 | STGGAPTFNVTVTMTAK | SEQ ID NO: 329 |
| | | | | EGVHGGLINK | SEQ ID NO: 94 |
| | | | | SSFFVNGLTLGGQK | SEQ ID NO: 326 |
| | | | | DSLLQDGEFTMDLR | SEQ ID NO: 75 |
| | | | | DSPSVWAAVPGK | SEQ ID NO: 76 |
| | | | | CYEMASHLR | SEQ ID NO: 51 |

The sixth group consists of LRPIs that are increased in the liver tissue of subjects with late liver response as compared with the liver tissue of subjects free from liver response. The amino acid sequences of tryptic digest peptides of these LRPIs identified by tandem mass spectrometry and database searching using the SEQUEST program are listed in Table XVIII.

Table XVIII. LRPIs Increased in Liver Tissue of Subjects Having Late Liver Response

TABLE XVIII

| | | | | Amino Acid Sequence of | |
|---|---|---|---|---|---|
| LRF# | LRPI# | pI | MW (Da) | Tryptic Digest Peptides | SEQ ID NO: |
| LRF-109 | LRPI-109.1 | 8.13 | 64092 | AMVENGGLVTSNPLR | SEQ ID NO: 25 |
| | | | | FFEEVNDPAK | SEQ ID NO: 131 |
| | | | | VPAENVLGEVGDGFK | SEQ ID NO: 378 |
| LRF-110 | LRPI-110.1 | 6.19 | 52175 | ALELEQELR | SEQ ID NO: 21 |
| | | | | KPFTEVIR | SEQ ID NO: 217 |
| | | | | ECIEAVIR | SEQ ID NO: 85 |
| | | | | LTEQVFNEAPGIR | SEQ ID NO: 254 |
| LRF-111 | LRPI-111.1 | 5.57 | 23697 | WNEEVEAYR | SEQ ID NO: 398 |
| | | | | LEPLGTELHK | SEQ ID NO: 229 |
| | | | | VKDFATVYVDAVK | SEQ ID NO: 369 |
| | | | | DYVSQFESSTLGK | SEQ ID NO: 83 |
| | | | | FGLYSDQMR | SEQ ID NO: 135 |
| LRF-112 | LRPI-112.1 | 4.99 | 49643 | IPVGPETLGR | SEQ ID NO: 205 |
| | | | | VALVYGQMNEPPGAR | SEQ ID NO: 359 |
| | | | | IMNVIGEPIDER | SEQ ID NO: 200 |
| | | | | VALTGLTVAEYFR | SEQ ID NO: 358 |
| LRF-112 | LRPI-112.2 | 4.99 | 49643 | TGEAIVDAALSALR | SEQ ID NO: 344 |
| | | | | LAAVDATVNQVLASR | SEQ ID NO: 220 |
| | | | | GSFSEQGINEFLR | SEQ ID NO: 170 |
| | | | | GSTAPVGGGSFPNITPR | SEQ ID NO: 172 |
| LRF-113 | LRPI-113.1 | 5.84 | 45749 | IVFSPEEAK | SEQ ID NO: 214 |
| | | | | ICNQVLVCER | SEQ ID NO: 189 |
| | | | | INFDSNSAYR | SEQ ID NO: 201 |
| LRF-114 | LRPI-114.1 | 6.10 | 183101 | EIEYEVVR | SEQ ID NO: 99 |
| | | | | GQNQPVLNITNR | SEQ ID NO: 167 |
| | | | | GLNSESVTEETLR | SEQ ID NO: 162 |
| LRF-115 | LRPI-115.1 | 5.85 | 162581 | SLLEGEGSSGGGGR | SEQ ID NO: 320 |
| | | | | QSLEASLAETEGR | SEQ ID NO: 300 |
| LRF-115 | LRPI-115.2 | 5.85 | 162581 | EIEYEVVR | SEQ ID NO: 99 |
| | | | | GLNSESVTEETLR | SEQ ID NO: 162 |
| LRF-119 | LRPI-119.1 | 6.86 | 46042 | AAHLCAEAALR | SEQ ID NO: 1 |
| LRF-119 | LRPI-119.2 | 6.86 | 46042 | MGFPEAASSFR | SEQ ID NO: 262 |
| | | | | VFVVGVGMTK | SEQ ID NO: 366 |
| LRF-119 | LRPI-119.3 | 6.86 | 46042 | LQHGSILGFPK | SEQ ID NO: 250 |
| | | | | DDGSWEVIEGYR | SEQ ID NO: 55 |
| | | | | MVEGFFDR | SEQ ID NO: 270 |
| LRF-120 | LRPI-120.1 | 8.25 | 35532 | GAAQNIIPASTGAAK | SEQ ID NO: 152 |
| | | | | PITIFQER | SEQ ID NO: 291 |
| | | | | LISWYDNEYGYSNR | SEQ ID NO: 238 |
| LRF-121 | LRPI-121.1 | 5.51 | 54245 | ETTVVWDK | SEQ ID NO: 118 |
| | | | | EGWVEQDPK | SEQ ID NO: 95 |
| | | | | EILDAMNR | SEQ ID NO: 100 |
| | | | | LGQLNIDISNIK | SEQ ID NO: 235 |
| | | | | AVLGPLVGAVDQGTSSTR | SEQ ID NO: 40 |
| | | | | TAELLSHHQVEIK | SEQ ID NO: 335 |
| | | | | FEPQINAEESEIR | SEQ ID NO: 130 |
| | | | | IPGNNNFVK | SEQ ID NO: 204 |
| LRF-122 | LRPI-122.1 | 9.04 | 11379 | MFAAFPTTK | SEQ ID NO: 261 |
| | | | | IGGHGGEYGEEALQR | SEQ ID NO: 194 |
| | | | | TYFSHIDVSPGSAQVK | SEQ ID NO: 356 |
| LRF-123 | LRPI-123.1 | 4.63 | 11809 | NMQDMVEDYR | SEQ ID NO: 282 |
| LRF-123 | LRPI-123.2 | 4.63 | 11809 | SLLEGEGSSGGGGR | SEQ ID NO: 320 |
| LRF-124 | LRPI-124.1 | 6.27 | 27026 | NVLIVEDIIDTGK | SEQ ID NO: 287 |
| | | | | VFIPHGLIMDR | SEQ ID NO: 364 |
| | | | | DLNHVCVISESGK | SEQ ID NO: 68 |
| LRF-126 | LRPI-126.1 | 5.68 | 59969 | NFDVGHVPIR | SEQ ID NO: 274 |
| LRF-127 | LRPI-127.1 | 5.02 | 49336 | AQIFANSVDNAR | SEQ ID NO: 30 |
| | | | | QSVESDIHGLR | SEQ ID NO: 301 |
| | | | | YETELAMR | SEQ ID NO: 404 |
| | | | | AQYEQLAQK | SEQ ID NO: 33 |
| | | | | IVLQIDNAR | SEQ ID NO: 215 |
| LRF-128 | LRPI-128.1 | 5.77 | 52350 | ATLEAAIADAEQR | SEQ ID NO: 36 |
| | | | | AQYEEIANR | SEQ ID NO: 32 |
| | | | | LEVDPNIQAVR | SEQ ID NO: 230 |
| | | | | LEGLTDEINFLR | SEQ ID NO: 228 |
| LRF-129 | LRPI-129.1 | 5.29 | 57645 | AAVEEGIVLGGGCALLR | SEQ ID NO: 3 |
| | | | | TLNDELEIIEGMK | SEQ ID NO: 348 |
| | | | | NAGVEGSLIVEK | SEQ ID NO: 272 |
| | | | | CEFQDAYVLLSEK | SEQ ID NO: 44 |
| | | | | CIPALDSLKPANEDQK | SEQ ID NO: 46 |
| LRF-130 | LRPI-130.1 | 6.21 | 57505 | STSIIATIGPASR | SEQ ID NO: 330 |
| | | | | EPPEAIWADDVDR | SEQ ID NO: 113 |
| LRF-131 | LRPI-131.1 | 5.16 | 12722 | MLSNENFEEYLR | SEQ ID NO: 264 |
| | | | | EFEEDLTGIDDR | SEQ ID NO: 91 |
| | | | | ALDVNVALR | SEQ ID NO: 20 |
| | | | | NYIMDFQVGK | SEQ ID NO: 290 |
| LRF-132 | LRPI-132.1 | 6.30 | 66705 | GVIALCIEDGSIHR | SEQ ID NO: 174 |
| | | | | ACALSIAESCRPGDK | SEQ ID NO: 4 |
| | | | | TGHSLLHTLYGR | SEQ ID NO: 345 |
| LRF-132 | LRPI-132.2 | 6.30 | 66705 | APQVSTPTLVEAAR | SEQ ID NO: 28 |
| | | | | RHPDYSVSLLLR | SEQ ID NO: 305 |
| LRF-133 | LRPI-133.1 | 6.30 | 27762 | EMNLSETAFIR | SEQ ID NO: 107 |
| LRF-134 | LRPI-134.1 | 6.02 | 58605 | AFHNEAQVNPER | SEQ ID NO: 10 |
| | | | | EQLAIAEFAR | SEQ ID NO: 115 |
| LRF-395 | LRPI-395.1 | 9.74 | 25549 | SHGQDYLVGNR | SEQ ID NO: 315 |
| LRF-395 | LRPI-395.2 | 9.74 | 25549 | SLHVYLEK | SEQ ID NO: 319 |
| LRF-397 | LRPI-397.1 | 5.74 | 68828 | CCTLPEAQR | SEQ ID NO: 43 |
| | | | | APQVSTPTLVEAAR | SEQ ID NO: 28 |
| LRF-399 | LRPI-399.1 | 5.83 | 67405 | CCTLPEAQR | SEQ ID NO: 43 |
| | | | | APQVSTPTLVEAAR | SEQ ID NO: 28 |
| | | | | LPCVEDYLSAILNR | SEQ ID NO: 246 |
| | | | | ECCHGDLLECADDR | SEQ ID NO: 84 |
| | | | | SIHTLFGDK | SEQ ID NO: 317 |
| LRF-401 | LRPI-401.1 | 7.13 | 39509 | ETEYNVR | SEQ ID NO: 117 |
| | | | | VMEETFSYLLGR | SEQ ID NO: 373 |
| | | | | DVDPGEHYIIK | SEQ ID NO: 80 |
| LRF-402 | LRPI-402.1 | 7.37 | 51294 | HGGTIPVVPTAEFQDR | SEQ ID NO: 183 |
| | | | | TFVVQGFGNVGLHSMR | SEQ ID NO: 343 |
| | | | | DIVHSGLAYTMER | SEQ ID NO: 67 |
| | | | | MVEGFFDR | SEQ ID NO: 270 |
| LRF-403 | LRPI-403.1 | 7.83 | 51921 | VLSIGDGIAR | SEQ ID NO: 372 |
| | | | | HALIIYDDLSK | SEQ ID NO: 181 |
| LRF-405 | LRPI-405.1 | 6.76 | 30546 | SYPGSQLDILIDQGK | SEQ ID NO: 332 |
| LRF-405 | LRPI-405.2 | 6.76 | 30546 | SLVNELTFTA | SEQ ID NO: 323 |
| | | | | ELVECFQK | SEQ ID NO: 105 |
| LRF-407 | LRPI-407.1 | 6.04 | 25363 | TVMGDFAQFVDK | SEQ ID NO: 353 |
| LRF-409 | LRPI-409.1 | 5.59 | 27762 | ISELDAFLK | SEQ ID NO: 210 |
| | | | | APLDIPVPDPVK | SEQ ID NO: 27 |
| | | | | LMVMEIR | SEQ ID NO: 245 |
| | | | | TENLLGSYFPK | SEQ ID NO: 340 |
| | | | | IEDGNNFGVAVQEK | SEQ ID NO: 191 |
| | | | | EPALNEANLSNLK | SEQ ID NO: 111 |

The seventh group consists of LRPIs that are decreased in the blood of subjects with early liver response as compared with the blood of subjects free from liver response. The amino acid sequences of tryptic digest peptides of these LRPIs identified by tandem mass spectrometry and database searching using the SEQUEST program are listed in Table XIX.

Table XIX. LRPIs Decreased in Serum/Plasma of Subjects Having Early Liver Response

TABLE XIX

| | | | | Amino Acid Sequences of | |
|---|---|---|---|---|---|
| LRF# | LRPI# | pI | MW (Da) | Tryptic Digest Peptides | SEQ ID NO: |
| LRF-156 | LRPI-156.1 | 4.93 | 54483 | MQHLEQTLTK | SEQ ID NO: 267 |
| | | | | FDHPFIF | SEQ ID NO: 128 |
| | | | | AVLTLDER | SEQ ID NO: 41 |
| | | | | VFNNDADLSGITEDAPLK | SEQ ID NO: 365 |
| | | | | RPFNPEHTR | SEQ ID NO: 306 |
| LRF-157 | LRPI-157.1 | 6.17 | 28245 | RSEVLESLNK | SEQ ID NO: 308 |
| | | | | AISYLISGYQR | SEQ ID NO: 19 |
| LRF-158 | LRPI-158.1 | 5.89 | 128039 | GVVSFPIR | SEQ ID NO: 177 |
| | | | | IHFLLNEDALK | SEQ ID NO: 195 |
| | | | | IEHSFEVK | SEQ ID NO: 192 |
| | | | | TVSWAVTPK | SEQ ID NO: 355 |
| LRF-159 | LRPI-159.1 | 6.09 | 138959 | AEDITHNGIVYTPK | SEQ ID NO: 9 |
| | | | | AISYLISGYQR | SEQ ID NO: 19 |
| LRF-160 | LRPI-160.1 | 5.09 | 42125 | QLDQQVEVFR | SEQ ID NO: 296 |
| | | | | LGNINTYADDLQNK | SEQ ID NO: 233 |
| | | | | ATIDQNLEDLR | SEQ ID NO: 35 |
| LRF-161 | LRPI-161.1 | 4.94 | 36692 | TANLGAGAAQPLR | SEQ ID NO: 337 |
| LRF-162 | LRPI-162.1 | 4.95 | 35503 | TANLGAGAAQPLR | SEQ ID NO: 337 |
| | | | | GWFEPLVEDMQR | SEQ ID NO: 178 |
| | | | | LGADMEDLR | SEQ ID NO: 231 |
| | | | | LGPLVEQGR | SEQ ID NO: 234 |
| | | | | DRLEEVR | SEQ ID NO: 73 |
| LRF-163 | LRPI-163.1 | 5.13 | 31133 | TANLGAGAAQPLR | SEQ ID NO: 337 |
| | | | | LGPLVEQGR | SEQ ID NO: 234 |
| | | | | NEVNTMLGQSTEELR | SEQ ID NO: 273 |
| LRF-164 | LRPI-164.1 | 5.31 | 40165 | VHQFFNVGLIQPGSVK | SEQ ID NO: 368 |
| | | | | SSMILDICTR | SEQ ID NO: 327 |
| | | | | SGSDEVQAGQER | SEQ ID NO: 314 |
| | | | | GQGTLSVVTVYHAK | SEQ ID NO: 166 |
| | | | | ISHSEEDCLSFK | SEQ ID NO: 211 |
| LRF-165 | LRPI-165.1 | 5.88 | 27988 | YYQTIEIPPK | SEQ ID NO: 412 |
| | | | | SDVDEDIIPEEDIISR | SEQ ID NO: 312 |
| LRF-166 | LRPI-166.1 | 5.65 | 38289 | SSMILDICTR | SEQ ID NO: 327 |
| | | | | SGSDEVQAGQER | SEQ ID NO: 314 |
| | | | | LCHNEMCR | SEQ ID NO: 226 |
| | | | | VYSYYNLEESCTR | SEQ ID NO: 396 |
| LRF-167 | LRPI-167.1 | 4.89 | 49621 | DHAVDLIQK | SEQ ID NO: 66 |
| LRF-168 | LRPI-168.1 | 5.1 | 73017 | SIEVFGQFQGK | SEQ ID NO: 316 |
| | | | | EEPSADALLPIDCR | SEQ ID NO: 89 |
| | | | | TENYEEQFEMFK | SEQ ID NO: 341 |
| | | | | VVEESELGR | SEQ ID NO: 393 |
| | | | | DGNTLTYYR | SEQ ID NO: 64 |
| | | | | AIEDYVNEFSAR | SEQ ID NO: 17 |
| LRF-169 | LRPI-169.1 | 5.62 | 45452 | LEPLGTELHK | SEQ ID NO: 229 |
| | | | | FGLYSDQMR | SEQ ID NO: 135 |
| LRF-170 | LRPI-170.1 | 6.03 | 103852 | SEDCFILDHGR | SEQ ID NO: 313 |
| | | | | HVVPNEVVVQR | SEQ ID NO: 187 |
| | | | | EPGLQIWR | SEQ ID NO: 112 |
| LRF-172 | LRPI-172.1 | 5.47 | 69633 | FNPVTGEVPPR | SEQ ID NO: 142 |
| LRF-173 | LRPI-173.1 | 6.38 | 68285 | NPVTSVDAAFR | SEQ ID NO: 284 |
| | | | | GECQSEGVLFFQGNR | SEQ ID NO: 154 |
| | | | | FNPVTGEVPPR | SEQ ID NO: 142 |
| LRF-174 | LRPI-174.1 | 6.37 | 73744 | NPVTSVDAAFR | SEQ ID NO: 284 |
| | | | | GECQSEGVLFFQGNR | SEQ ID NO: 154 |
| | | | | FNPVTGEVPPR | SEQ ID NO: 142 |
| LRF-175 | LRPI-175.1 | 5.4 | 86193 | EVQLVESGGGLVQPGR | SEQ ID NO: 121 |
| LRF-175 | LRPI-175.2 | 5.4 | 86193 | LICEATNFSPK | SEQ ID NO: 236 |
| | | | | HPPAVYLLPPAR | SEQ ID NO: 185 |
| | | | | DAFSGPAPR | SEQ ID NO: 53 |
| LRF-176 | LRPI-176.1 | 4.62 | 51617 | IFFYDSENPPGSEVLR | SEQ ID NO: 193 |
| | | | | LLIGTVFHR | SEQ ID NO: 241 |
| LRF-177 | LRPI-177.1 | 7.08 | 19005 | YWGVASFLQR | SEQ ID NO: 410 |
| | | | | DPNGLTPETR | SEQ ID NO: 70 |
| | | | | QRQEELCLER | SEQ ID NO: 299 |
| LRF-178 | LRPI-178.1 | 7.54 | 104678 | WEYCDIPR | SEQ ID NO: 397 |
| LRF-179 | LRPI-179.1 | 6.84 | 108902 | WEYCDIPR | SEQ ID NO: 397 |
| | | | | YVNWIER | SEQ ID NO: 409 |
| | | | | CEGETDFICR | SEQ ID NO: 45 |
| LRF-181 | LRPI-181.1 | 6.06 | 43714 | FVIGGPQGDAGLTGR | SEQ ID NO: 148 |
| LRF-182 | LRPI-182.1 | 5.76 | 68203 | APQVSTPTLVEAAR | SEQ ID NO: 28 |
| LRF-183 | LRPI-183.1 | 5.39 | 103579 | DGSFGAWLHR | SEQ ID NO: 65 |
| LRF-184 | LRPI-184.1 | 7.55 | 77168 | DSAFGLLR | SEQ ID NO: 74 |
| LRF-184 | LRPI-184.2 | 7.55 | 77168 | DQYELLCLDNTR | SEQ ID NO: 72 |

The eighth group consists of LRPIs that are increased in the blood of subjects with early liver response as compared with the blood of subjects free from liver response. The amino acid sequences of tryptic digest peptides of these LRPIs identified by tandem mass spectrometry and database searching using the SEQUEST program are listed in Table XX.

Table XX. LRPIs Increased in Blood of Subjects Having Early Liver Response

TABLE XX

| | | | | Amino Acid Sequences of | |
|---|---|---|---|---|---|
| LRF# | LRPI# | pI | MW (Da) | Tryptic Digest Peptides | SEQ ID NO: |
| LRF-217 | LRPI-217.1 | 5.06 | 44232 | GYSFTTTAER | SEQ ID NO: |
| 180 | | | | | |
| | | | | AGFAGDDAPR | SEQ ID NO: 14 |
| | | | | SYELPDGQVITIGNER | SEQ ID NO: 331 |
| LRF-217 | LRPI-217.3 | 5.06 | 44232 | SSMILDICTR | SEQ ID NO: 327 |
| | | | | VYSYYNLEESCTR | SEQ ID NO: 396 |
| LRF-218 | LRPI-218.1 | 5.42 | 23154 | LEPLGTELHK | SEQ ID NO: 229 |
| | | | | FGLYSDQMR | SEQ ID NO: 135 |
| LRF-219 | LRPI-219.1 | 4.95 | 34894 | TANLGAGAAQPLR | SEQ ID NO: 337 |
| | | | | GWFEPLVEDMQR | SEQ ID NO: 178 |
| LRF-220 | LRPI-220.1 | 6.99 | 78836 | MLFNQQELFGR | SEQ ID NO: 263 |
| LRF-221 | LRPI-221.1 | 5.29 | 38416 | SSMILDICTR | SEQ ID NO: 327 |
| | | | | SGSDEVQAGQER | SEQ ID NO: 314 |
| | | | | ACEPGVDYVYK | SEQ ID NO: 5 |
| | | | | VTIKPAPETAK | SEQ ID NO: 388 |
| | | | | VYSYYNLEESCTR | SEQ ID NO: 396 |
| | | | | ISHSEEDCLSFK | SEQ ID NO: 211 |
| LRF-222 | LRPI-222.1 | 4.8 | 37589 | AGEYLEASYLNLQR | SEQ ID NO: 13 |
| | | | | DFDSVPPVVR | SEQ ID NO: 62 |
| | | | | LEEPYLTK | SEQ ID NO: 227 |
| | | | | RQEALELIK | SEQ ID NO: 307 |
| LRF-223 | LRPI-223.1 | 7.43 | 28246 | LLSTLCSADVCQCAEGK | SEQ ID NO: 243 |
| LRF-224 | LRPI-224.1 | 7.14 | 28269 | VQYTVCIWR | SEQ ID NO: 384 |
| | | | | FACYYPR | SEQ ID NO: 124 |
| | | | | CSVFYAAPTK | SEQ ID NO: 50 |
| LRF-225 | LRPI-225.1 | 6.25 | 90963 | GVISWGSGCGDR | SEQ ID NO: 175 |
| LRF-226 | LRPI-226.1 | 6.02 | 100636 | SEDCFILDHGR | SEQ ID NO: 313 |
| | | | | HVVPNEVVVQR | SEQ ID NO: 187 |
| | | | | EPGLQIWR | SEQ ID NO: 112 |
| LRF-227 | LRPI-227.1 | 5.19 | 82775 | NPVTSVDAAFR | SEQ ID NO: 284 |
| | | | | DYFISCPGR | SEQ ID NO: 82 |
| | | | | FNPVTGEVPPR | SEQ ID NO: 142 |
| LRF-228 | LRPI-228.1 | 6.3 | 73388 | NPVTSVDAAFR | SEQ ID NO: 284 |
| | | | | FNPVTGEVPPR | SEQ ID NO: 142 |
| LRF-230 | LRPI-230.1 | 5.13 | 121204 | VRPEQLVK | SEQ ID NO: 385 |
| | | | | ADTVQEATFQVELPR | SEQ ID NO: 7 |
| | | | | VTISLLSLDDPQR | SEQ ID NO: 389 |
| LRF-231 | LRPI-231.1 | 5.2 | 120973 | VLGIDYPATR | SEQ ID NO: 371 |
| | | | | VRPEQLVK | SEQ ID NO: 385 |
| | | | | VTISLLSLDDPQR | SEQ ID NO: 389 |
| | | | | FAHTVVTSR | SEQ ID NO: 126 |
| LRF-232 | LRPI-232.1 | 4.72 | 65573 | NLHVSQVVHK | SEQ ID NO: 278 |
| | | | | EQPILSEFQEK | SEQ ID NO: 116 |
| | | | | DSTMEEILEGLK | SEQ ID NO: 77 |
| | | | | DEELSCSVLELK | SEQ ID NO: 57 |
| | | | | ALYQAEAFVADFK | SEQ ID NO: 24 |
| LRF-232 | LRPI-232.2 | 4.72 | 65573 | EVLPELGIK | SEQ ID NO: 120 |
| | | | | EQPILSEFQEK | SEQ ID NO: 116 |
| | | | | DSTMEEILEGLK | SEQ ID NO: 77 |
| | | | | DEELSCSVLELK | SEQ ID NO: 57 |
| | | | | FSISTDYSLK | SEQ ID NO: 146 |
| LRF-234 | LRPI-234.1 | 5.11 | 30589 | SITLFVQEDR | SEQ ID NO: 318 |
| | | | | GDVNDNFQGVLQNVR | SEQ ID NO: 153 |
| LRF-235 | LRPI-235.1 | 6.9 | 88853 | KPVDQYEDCYLAR | SEQ ID NO: 219 |
| | | | | DQYELLCLDNTR | SEQ ID NO: 72 |
| LRF-236 | LRPI-236.1 | 6.09 | 39155 | TADGSWEPFASGK | SEQ ID NO: 334 |
| LRF-237 | LRPI-237.1 | 5.45 | 49032 | YCSSQIDAEMR | SEQ ID NO: 402 |
| | | | | VCSQYAAYGK | SEQ ID NO: 361 |
| | | | | ELPEHTLK | SEQ ID NO: 104 |
| | | | | SLSLILYSR | SEQ ID NO: 322 |
| | | | | RTQVPEVFLSK | SEQ ID NO: 309 |

The ninth group consists of LRPIs that are decreased in the blood of subjects with full manifestation liver response as compared with the blood of subjects free from liver response. The amino acid sequences of tryptic digest peptides of these LRPIs identified by tandem mass spectrometry and database searching using the SEQUEST program are listed in Table XXI.

Table XXI. LRPIs Decreased in Blood of Subjects Having Full Manifestation Liver Response

TABLE XXI

| | | | | Amino Acid Sequences of | |
|---|---|---|---|---|---|
| LRF# | LRPI# | pI | MW (Da) | Tryptic Digest Peptides | SEQ ID NO: |
| LRF-256 | LRPI-256.1 | 5.06 | 38779 | VSGSGCVYLQTSLK | SEQ ID NO: 386 |
| | | | | NLKPAPVK | SEQ ID NO: 279 |
| | | | | LQDQSNIQR | SEQ ID NO: 248 |
| | | | | TEVNTNHVLIYIEK | SEQ ID NO: 342 |
| | | | | VNTLPLNFDK | SEQ ID NO: 377 |
| | | | | LADLPGNYITK | SEQ ID NO: 221 |
| | | | | YGAATFTK | SEQ ID NO: 405 |
| | | | | MVSGFIPVKPSVK | SEQ ID NO: 271 |
| LRF-257 | LRPI-257.1 | 5.69 | 35827 | LQDQSNIQR | SEQ ID NO: 248 |
| | | | | VNTLPLNFDK | SEQ ID NO: 377 |
| | | | | LADLPGNYITK | SEQ ID NO: 221 |
| LRF-258 | LRPI-258.1 | 5.61 | 35868 | LQDQSNIQR | SEQ ID NO: 248 |
| | | | | VNTLPLNFDK | SEQ ID NO: 377 |
| LRF-259 | LRPI-259.1 | 5.32 | 23373 | WNEEVEAYR | SEQ ID NO: 398 |
| | | | | LEPLGTELHK | SEQ ID NO: 229 |
| LRF-260 | LRPI-260.1 | 4.97 | 46213 | QLDQQVEVFR | SEQ ID NO: 296 |
| | | | | ATIDQNLEDLR | SEQ ID NO: 35 |
| LRF-261 | LRPI-261.1 | 5.03 | 41400 | QLDQQVEVFR | SEQ ID NO: 296 |
| LRF-262 | LRPI-262.1 | 4.93 | 31680 | MEEQTQQIRDRLEEVR | SEQ ID NO: 260 |
| | | | | FWDYLR | SEQ ID NO: 151 |
| LRF-263 | LRPI-263.1 | 5.39 | 62144 | WSPELPVCAR | SEQ ID NO: 400 |
| | | | | ITCPPPPIPK | SEQ ID NO: 212 |
| | | | | AVFGCHETYK | SEQ ID NO: 39 |
| | | | | ATVLYQGQR | SEQ ID NO: 37 |
| LRF-264 | LRPI-264.1 | 6.78 | 53959 | ITCPPPPIPK | SEQ ID NO: 212 |
| | | | | AVFGCHETYK | SEQ ID NO: 39 |
| | | | | ATVLYQGQR | SEQ ID NO: 37 |
| LRF-265 | LRPI-265.1 | 6.83 | 28900 | NSIAYLDEETGR | SEQ ID NO: 286 |
| LRF-266 | LRPI-266.1 | 5.38 | 38357 | SSMILDICTR | SEQ ID NO: 327 |
| | | | | ACEPGVDYVYK | SEQ ID NO: 5 |
| | | | | VYSYYNLEESCTR | SEQ ID NO: 396 |
| | | | | ISHSEEDCLSFK | SEQ ID NO: 211 |
| LRF-270 | LRPI-270.1 | 5.57 | 15056 | LCEAHGITR | SEQ ID NO: 225 |
| | | | | NGETFQLMVLYGR | SEQ ID NO: 276 |
| | | | | ELYLVAYK | SEQ ID NO: 106 |
| LRF-271 | LRPI-271.1 | 4.67 | 51056 | IFFYDSENPPGSEVLR | SEQ ID NO: 193 |

The tenth group consists of LRPIs that are increased in the blood of subjects with full manifestation liver response as compared with the blood of subjects free from liver response. The amino acid sequences of tryptic digest peptides of these LRPIs identified by tandem mass spectrometry and database searching using the SEQUEST program are listed in Table XXII.

Table XXII. LRPIs Increased in Blood of Subjects Having Full Manifestation Liver Response

TABLE XXII
LRPIs Increased in Blood of Subjects Having
Full Manifestation Liver Response

| | | | | Amino Acid | |
|---|---|---|---|---|---|
| | | | | Sequences of | |
| | | | | Tryptic Digest | |
| LRF# | LRPI# | pI | MW (Da) | Peptides | SEQ ID NO: |
| LRF-294 | LRPI-294.1 | 4.95 | 32192 | TANLGAGAAQPLR | SEQ ID NO: 337 |
| | | | | GWFEPLVEDMQR | SEQ ID NO: 178 |
| | | | | LGPLVEQGR | SEQ ID NO: 234 |
| | | | | FWDYLR | SEQ ID NO: 151 |
| LRF-295 | LRPI-295.1 | 7.31 | 56943 | WSPELPVCAR | SEQ ID NO: 400 |
| | | | | ITCPPPPIPK | SEQ ID NO: 212 |
| | | | | ATVLYQGQR | SEQ ID NO: 37 |
| LRF-296 | LRPI-296.1 | 7.21 | 28524 | VQYTVCIWR | SEQ ID NO: 384 |
| | | | | CSVFYAAPTK | SEQ ID NO: 50 |
| LRF-297 | LRPI-297.1 | 6.8 | 39504 | ILVDYDEK | SEQ ID NO: 199 |
| | | | | GNLTDLETNGVR | SEQ ID NO: 164 |
| | | | | MPINEPAPGR | SEQ ID NO: 266 |
| | | | | TEDIITTIR | SEQ ID NO: 339 |
| LRF-298 | LRPI-298.1 | 7.08 | 39510 | ASSVVVSGTPIR | SEQ ID NO: 34 |
| | | | | FGEPIPISK | SEQ ID NO: 134 |
| | | | | AIDVGQGQTR | SEQ ID NO: 16 |
| LRF-300 | LRPI-300.1 | 5.25 | 91060 | YTVCDSVR | SEQ ID NO: 408 |
| | | | | ENLSPPLGECLLER | SEQ ID NO: 110 |
| | | | | ELLDSYIDGR | SEQ ID NO: 103 |
| | | | | EECSIPVCGQEGR | SEQ ID NO: 88 |
| LRF-300 | LRPI-300.2 | 5.25 | 91060 | DAFSGPAPR | SEQ ID NO: 53 |
| LRF-301 | LRPI-301.1 | 5.67 | 81660 | LICEATNFSPK | SEQ ID NO: 236 |
| | | | | HPPAVYLLPPAR | SEQ ID NO: 185 |
| LRF-303 | LRPI-303.1 | 4.9 | 75356 | DGAETLYSFK | SEQ ID NO: 63 |
| | | | | LLNSCEYK | SEQ ID NO: 242 |
| | | | | EDFPFLR | SEQ ID NO: 86 |
| LRF-305 | LRPI-305.1 | 6.2 | 33596 | ENFSCLTR | SEQ ID NO: 109 |
| LRF-306 | LRPI-306.1 | 6.67 | 28449 | FPAMSDAYDR | SEQ ID NO: 143 |

The eleventh group consists of LRPIs that are decreased in the blood of subjects with late liver response as compared with the blood of subjects free from liver response. The amino acid sequences of tryptic digest peptides of these LRPIs identified by tandem mass spectrometry and database searching using the SEQUEST program are listed in Table XXIII.

Table XXIII. LRPIs Decreased in Blood of Subjects Having Late Liver Response

TABLE XXIII
LRPIs Decreased in Blood of Subjects Having Late Liver Response

| | | | | Amino Acid | |
|---|---|---|---|---|---|
| | | | | Sequences of | |
| LRF# | LRPI# | pI | MW (Da) | Tryptic Digest Peptides | SEQ ID NO: |
| LRF-328 | LRPI-328.1 | 6.54 | 139228 | TVSWAVTPK | SEQ ID NO: 355 |
| | | | | RSEVLESLNK | SEQ ID NO: 308 |
| LRF-329 | LRPI-329.1 | 5.94 | 33501 | VNTLPLNFDK | SEQ ID NO: 377 |
| | | | | LADLPGNYITK | SEQ ID NO: 221 |
| LRF-331 | LRPI-331.1 | 5.97 | 140431 | GVVSFPIR | SEQ ID NO: 177 |
| | | | | GSIFNSGSHVLPLEQGK | SEQ ID NO: 171 |
| | | | | VPDTITEWK | SEQ ID NO: 380 |
| | | | | TVSWAVTPK | SEQ ID NO: 355 |
| | | | | RSEVLESLNK | SEQ ID NO: 308 |
| LRF-332 | LRPI-332.1 | 5.85 | 61591 | ITCPPPPIPK | SEQ ID NO: 212 |
| | | | | AVFGCHETYK | SEQ ID NO: 39 |
| | | | | ATVLYQGQR | SEQ ID NO: 37 |
| | | | | EHSSLAFWK | SEQ ID NO: 96 |
| LRF-334 | LRPI-334.1 | 5.85 | 101552 | HVVPNEVVVQR | SEQ ID NO: 187 |
| | | | | AGALNSNDAFVLK | SEQ ID NO: 12 |
| LRF-335 | LRPI-335.1 | 6.56 | 71935 | DYFISCPGR | SEQ ID NO: 82 |
| | | | | FNPVTGEVPPR | SEQ ID NO:142 |

The twelfth group consists of LRPIs that are increased in the blood of subjects with late liver response as compared with the blood of subjects free from liver response. The amino acid sequences of tryptic digest peptides of these LRPIs identified by tandem mass spectrometry and database searching using the SEQUEST program are listed in Table XXIV.

Table XXIV. LRPIs Increased in Blood of Subjects Having Late Liver Response

TABLE XXIV
LRPIs Increased in Blood of Subjects Having Late Liver Response

| | | | | Amino Acid | |
|---|---|---|---|---|---|
| | | | | Sequences of | |
| LRF# | LRPI# | pI | MW (Da) | Tryptic Digest Peptides | SEQ ID NO: |
| LRF-356 | LRPI-356.1 | 6.13 | 29252 | AQQEQELAADAFK | SEQ ID NO: 31 |
| LRF-358 | LRPI-358.1 | 5.33 | 36864 | VSGSGCVYLQTSLK | SEQ ID NO: |
| | | | | | 386 |
| | | | | NLKPAPVK | SEQ ID NO: 279 |
| | | | | LQDQSNIQR | SEQ ID NO: 248 |
| | | | | TEVNTNHVLIYIEK | SEQ ID NO: 342 |
| | | | | VNTLPLNFDK | SEQ ID NO: 377 |
| | | | | LADLPGNYITK | SEQ ID NO: 221 |
| | | | | YGAATFTK | SEQ ID NO: 405 |
| | | | | KLQDQSNIQR | SEQ ID NO: 216 |
| | | | | MVSGFIPVKPSVK | SEQ ID NO: 271 |
| LRF-359 | LRPI-359.1 | 5.08 | 33850 | TANLGAGAAQPLR | SEQ ID NO: 337 |
| | | | | LGPLVEQGR | SEQ ID NO: 234 |
| LRF-360 | LRPI-360.1 | 4.86 | 36619 | TANLGAGAAQPLR | SEQ ID NO: 337 |
| | | | | LGADMEDLR | SEQ ID NO: 231 |
| | | | | LGPLVEQGR | SEQ ID NO: 234 |
| LRF-361 | LRPI-361.1 | 5.07 | 31110 | TANLGAGAAQPLR | SEQ ID NO: 337 |
| | | | | GWFEPLVEDMQR | SEQ ID NO: 178 |
| | | | | LGPLVEQGR | SEQ ID NO: 234 |
| | | | | LGADMEDLR | SEQ ID NO: 231 |
| | | | | NEVNTMLGQSTEELR | SEQ ID NO: 273 |
| | | | | MEEQTQQIR | SEQ ID NO: 259 |
| | | | | QWANLMEK | SEQ ID NO: 304 |
| LRF-363 | LRPI-363.1 | 5.13 | 43976 | SSMILDICTR | SEQ ID NO: 327 |
| | | | | VYSYYNLEESCTR | SEQ ID NO: 396 |
| LRF-364 | LRPI-364.1 | 5.48 | 38709 | SSMILDICTR | SEQ ID NO: 327 |
| | | | | SGSDEVQAGQER | SEQ ID NO: 314 |
| | | | | ACEPGVDYVYK | SEQ ID NO: 5 |
| | | | | VTIKPAPETAK | SEQ ID NO: 388 |
| | | | | VYSYYNLEESCTR | SEQ ID NO: 396 |
| LRF-365 | LRPI-365.1 | 5.71 | 72892 | NPVTSVDAAFR | SEQ ID NO: 284 |
| | | | | GECQSEGVLFFQGNR | SEQ ID NO: 154 |
| | | | | YYCFQGNK | SEQ ID NO: 411 |
| | | | | DYFISCPGR | SEQ ID NO: 82 |
| | | | | FNPVTGEVPPR | SEQ ID NO: 142 |
| LRF-366 | LRPI-366.1 | 4.8 | 48783 | FNPVTGEVPPR | SEQ ID NO: 142 |
| LRF-367 | LRPI-367.1 | 4.78 | 49587 | DYFISCPGR | SEQ ID NO: 82 |
| | | | | FNPVTGEVPPR | SEQ ID NO: 142 |
| LRF-368 | LRPI-368.1 | 4.93 | 69794 | DGAETLYSFK | SEQ ID NO: 63 |
| | | | | DAEEAATGECTTTLGK | SEQ ID NO: 52 |
| | | | | LLNSCEYK | SEQ ID NO: 242 |
| | | | | EDFPFLR | SEQ ID NO: 86 |
| | | | | YNAELESGNQFLLYR | SEQ ID NO: 406 |
| LRF-369 | LRPI-369.1 | 5.25 | 19817 | WPEPVFGR | SEQ ID NO: 399 |
| LRF-370 | LRPI-370.1 | 5.32 | 25447 | DEIIWLLR | SEQ ID NO: 58 |
| LRF-371 | LRPI-371.1 | 4.85 | 112218 | EQPILSEFQEK | SEQ ID NO: 116 |
| | | | | IAELFSDLEER | SEQ ID NO: 188 |
| | | | | FSISTDYSLK | SEQ ID NO: 146 |
| LRF-372 | LRPI-372.1 | 6.39 | 85568 | TVLPADGPR | SEQ ID NO: 352 |
| | | | | DQYELLCLDNTR | SEQ ID NO: 72 |
| LRF-373 | LRPI-373.1 | 5.63 | 12297 | TAESGELHGLTTDEK | SEQ ID NO: 336 |
| | | | | TADGSWEPFASGK | SEQ ID NO: 334 |

Those skilled in the art will understand, based upon the present description, that a given LRPI can be described according to the data provided for that LRPI in Tables XIII to Table XXIV, and according to the data provided for the corresponding LRF for that LRPI in Tables I to Table XII. The LRPI is a protein comprising a peptide sequence described for that LRPI (such as a plurality of, or all of, the peptide sequences described for that LRPI) and has a pI of about the value stated for the corresponding LRF for that LRPI (within 10%, or within 5%, or within 1% of the stated value) and has a MW of about the value stated for the corresponding LRF for that LRPI (within 10%, or within 5%, or within 1% of the stated value).

In one embodiment, a biological sample such as liver tissue from a subject is analyzed for quantitative detection of one or more of the LRPIs as defined in any one of Tables XIII, XV or XVII or any suitable combination, wherein a decreased abundance of the LRPI or LRPIs (or any suitable combination) in the liver tissue from the subject relative to liver tissue from a subject or subjects free from liver response (e.g., a control sample or a previously determined reference range) indicates the presence of liver response.

In another embodiment of the invention, a biological sample such as liver tissue from a subject is analyzed for quantitative detection of one or more of the LRPIs as defined in Tables XIV, XVI and XVTII or any suitable combination, wherein an increased abundance of the LRPI or LRPIs (or any suitable combination) in the liver tissue from the subject relative to liver tissue from a subject or subjects free from liver response (e.g., a control sample or a previously determined reference range) indicates the presence of liver response.

In a further embodiment, a biological sample such as liver tissue from a subject is analyzed for quantitative detection of (a) one or more LRPIs, or any suitable combination, whose decreased abundance indicates the presence of liver response, e.g. the LRPIs as defined in any one of Tables XIII, XV or XVII and (b) one or more LRPIs, or any suitable combination, whose increased abundance indicates the presence of liver response, e.g. the LRPIs as defined in any one of Tables XIV, XVI or XVIII.

In another embodiment, a biological sample such as blood or serum/plasma from a subject is analyzed for quantitative detection of one or more of the LRPIs as defined in any one of Tables XIX, XXI or XXIII, or any suitable combination, wherein a decreased abundance of the LRPI or LRPIs (or any suitable combination) in the blood or serum/plasma from the subject relative to blood or serum/plasma from a subject or subjects free from liver response (e.g., a control sample or a previously determined reference range) indicates the presence of liver response.

In another embodiment, a biological sample such as blood or serum/plasma from a subject is analyzed for quantitative detection of one or more of the LRPIs as defined in any one of Tables XX, XXII or XXIV or any suitable combination, wherein an increased abundance of the LRPI or LRPIs (or any suitable combination) in the blood or serum/plasma from the subject relative to blood or serum/plasma from a subject or subjects free from liver response (e.g., a control sample or a previously determined reference range) indicates the presence of liver response.

In a further embodiment, a biological sample such as blood or serum/plasma from a subject is analyzed for quantitative detection of (a) one or more LRPIs, or any suitable combination, whose decreased abundance indicates the presence of liver response, e.g. the LRPIs as defined in any one of Tables XIX, XXI or XXIII; and (b) one or more LRPIs, or any suitable combination, whose increased abundance indicates the presence of liver response, e.g. the LRPIs as defined in any one of Tables XX, XXII or XXIV.

In yet a further embodiment, a biological sample such as blood or liver from a subject is analyzed for quantitative detection of one or more LRPIs and one or more previously known biomarkers of liver response (e.g., alpha-fetoprotein, alanine aminotransferase). In accordance with this embodiment, the abundance of each LRPI and known biomarker relative to a control or reference range indicates whether a subject has liver response.

Preferably, the abundance of an LRPI is normalized to an Expression Reference Protein Isoform (ERPI). ERPIs can be identified by amino acid sequence characterization of ERFs, which are described above, and which may be accomplished using the methods and apparatuses described herein

Preferred LRPIs include:

LRPI-1.1, LRPI-2.1, LRPI-3.1, LRPI-4.1, LRPI-6.1, LRPI-8.1, LRPI-9.1, LRPI-10.1, LRPI-11.1, LRPI-11.2, LRPI-12.1, LRPI-13.1, LRPI-14.1, LRPI-15.1,

LRPI-16.1, LRPI-17.1, LRPI-17.2, LRPI-18.1, LRPI-20.1, LRPI-21.1, LRPI-22.1, LRPI-23.1, LRPI-24.1, LRPI-25.1, LRPI-26.1, LRPI-27.1, LRPI-28.1, LRPI-29.1,

LRPI-30.1, LRPI-31.1, LRPI-32.1, LRPI-36.1, LRPI-38. 1, LRPI-53.1,

LRPI-54.1, LRPI-56.1, LRPI-56.2, LRPI-60.1, LRPI-61.1, LRPI-63.1, LRPI-64.1,

LRPI-66.1, LRPI-67.1, LRPI-69.1, LRPI-70. 1, LRPI-71.1, LRPI-72.1, LRPI-73.1,

LRPI-74.1, LRPI-75.1, LRPI-75.2, LRPI-76.1, LRPI-77.1, LRPI-78.1, LRPI-79.1,

LRPI-79.2, LRPI-80.1, LRPI-81.1, LRPI-83.1, LRPI-84.1, LRPI-85.1, LRPI-87.1,

LRPI-88.1, LRPI-89.1, LRPI-90.1, LRPI-91.1, LRPI-92. 1, LRPI-107.1,

LRPI-109.1, LRPI-110.1, LRPI-111.1, LRPI-112.1, LRPI-1 12.2, LRPI-113.1,

LRPI-114.1, LRPI-115.1, LRPI-115.2, LRPI-119.1, LRPI-119.2, LRPI-119.3,

LRPI-120.1, LRPI-121.1, LRPI-122.1, LRPI-123.1, LRPI-123.2, LRPI-124.1,

LRPI-126.1, LRPI-127.1, LRPI-128.1, LRPI-129.1, LRPI-130.1, LRPI-131.1,

LRPI-132.1, LRPI-132.2, LRPI-133.1, LRPI-134.1, LRPI-156.1, LRPI-157.1,

LRPI-158.1, LRPI-159.1, LRPI-160.1, LRPI-161.1, LRPI-162.1, LRPI-163.1,

LRPI-164.1, LRPI-165.1, LRPI-166.1, LRPI-167.1, LRPI-168.1, LRPI-169.1,

LRPI-172.1, LRPI-173.1, LRPI-174.1, LRPI-175.1, LRPI-175.2, LRPI-176.1,

LRPI-177.1, LRPI-178.1, LRPI-179.1, LRPI-182.1, LRPI-183.1, LRPI-184.1,

LRPI-217.1, LRPI-217.3, LRPI-218.1, LRPI-219.1, LRPI-220.1, LRPI-221.1,

LRPI-222.1, LRPI-223.1, LRPI-224.1, LRPI-225.1, LRPI-226.1, LRPI-227.1,

LRPI-228.1, LRPI-230.1, LRPI-231.1, LRPI-232.1, LRPI-232.2, LRPI-234.1,

LRPI-236.1, LRPI-237.1, LRPI-256.1, LRPI-257.1, LRPI-258.1, LRPI-259.1,

LRPI-260.1, LRPI-261.1, LRPI-262.1, LRPI-263.1, LRPI-264.1, LRPI-265.1,

LRPI-270.1, LRPI-271.1, LRPI-294.1, LRPI-295.1, LRPI-296.1, LRPI-297.1,

LRPI-298.1, LRPI-300.1, LRPI-300.2, LRPI-301.1, LRPI-303.1, LRPI-305.1,

LRPI-328.1, LRPI-329.1, LRPI-331.1, LRPI-332.1, LRPI-334.1, LRPI-335.1,

LRPI-356.1, LRPI-358.1, LRPI-359.1, LRPI-360.1, LRPI-361.1, LRPI-363.1,

LRPI-364.1, LRPI-365.1, LRPI-366.1, LRPI-367.1, LRPI-368.1, LRPI-369.1,

LRPI-370.1, LRPI-371.1, LRPI-373.1, LRPI-386.1, LRPI-387.1, LRPI-400.1,

LRPI-400.2, LRPI-389.1, LRPI-390.1, LRPI-391.1, LRPI-392.1, LRPI-393.1,

LRPI-394.1, LRPI-394.2, LRPI-395.1, LRPI-395.2, LRPI-397.1, LRPI-399.1,

LRPI-401.1, LRPI-402.1, LRPI-403.1, LRPI-405.1, LRPI-405.2, LRPI-407.1, LRPI-409.1, LRPI-410.1

As shown above, the LRPIs described herein include previously unknown proteins, as well as isoforms of known proteins where the isoforms were not previously known to be associated with liver response. For each LRPI, the present invention additionally provides: (a) a preparation comprising the isolated LRPI; (b) a preparation comprising one or more fragments of the LRPI; and (c) LRPI binding agents. It should be noted that preparations just described that contain or are based on multiple LRPIs. As used herein, an LRPI is “isolated” when it is present in a preparation that is substantially free of other proteins, i.e., a preparation in which less than 10% (or less than 5%, or less than 1%) of the total protein present is other protein(s). An “other protein” is a protein or protein isoform having a significantly different pI or MW from those of the isolated LRPI, as determined by 2D electrophoresis. As used herein, a “significantly different” pI or MW is one that permits the other protein to be resolved from the LRPI on 2D electrophoresis, which may be performed according to the Reference Protocol.

In one embodiment, an isolated protein is provided, that comprises a peptide with the amino acid sequence identified in Tables XIII to XXIV for an LRPI, said protein having a pI and MW within 10% (particularly within 5%, or within 1%) of the values identified in Tables I to XII for the corresponding LRF for that LRPI.

The LRPIs of the invention can be qualitatively or quantitatively detected by any method known to those skilled in the art, including but not limited to the technology specifically described herein, as well as kinase assays, enzyme assays, binding assays and other functional assays, immunoassays, and western blotting. In one embodiment of the invention, the LRPIs are separated on a 2-D gel by virtue of their MWs and pIs and are visualized by staining the gel, e.g. with an indicator or dye. In another embodiment, the LRPIs are stained with a fluorescent dye and imaged with a fluorescence scanner. Sypro Red (Molecular Probes, Inc., Eugene, Oreg.) is a suitable dye for this purpose. Alternative dyes are described in U.S. Ser. No. 09/412,168, filed on Oct. 5, 1999, and incorporated herein by reference in its entirety.

Alternatively, LRPIs can be detected in an immunoassay. In one embodiment, an immunoassay is performed by contacting a sample under test with an anti-LRPI antibody under-conditions such that immunospecific binding can occur if the LRPI is present, and detecting or measuring the amount of immunospecific binding by the antibody. Anti-LRPI antibodies can be produced by the methods and techniques described herein; examples of such antibodies known in the art which have been reported to recognize a protein having amino acid sequence corresponding to a sequence of an LRPI, or which have been reported to recognize a protein named in the database selected by searching with the LRPI sequence are set forth in Table XXV. These antibodies shown in Table XXV are already reported to bind to the protein of which the LRPI is itself predicted to be a family member. Particularly, the anti-LRPI antibody preferentially binds to the LRPI rather than to other isoforms of the same protein. In a particular embodiment, the anti-LRPI antibody binds to the LRPI with at least 2-fold greater affinity, more particularly at least 5-fold greater affinity, still more particularly at least 10-fold greater affinity, than to said other isoforms of the same protein. The skilled artisan can readily assess and determine the ability of the noted antibody to recognize or bind to the LRPI and the specificity of such binding or recognition. When the antibodies shown in Table XXV do not display the required preferential selectivity for the target LRPI, one skilled in the art can generate additional antibodies by using the LRPI itself for the generation of such antibodies.

LRPIs can be transferred from a gel to a suitable membrane (e.g. a PVDF membrane) and subsequently probed in suitable assays that include, without limitation, competitive and non-competitive assay systems using techniques such as western blots and “sandwich” immunoassays using anti-LRPI antibodies as described herein, e.g., the antibodies identified in Table XXV, or others raised against the LRPIs of interest as those skilled in the art will appreciate based on the present description. The immunoblots can be used to identify those anti-LRPI antibodies displaying the selectivity required to immuno-specifically differentiate an LRPI from other isoforms encoded by the same gene. For example, one skilled in the art can identify anti-LRPI antibodies in catalogues of commercially available antibodies. Some examples of companies that supply antibodies include Accurate Chemical & Scientific Corporation, 300 Shames Drive, Westbury, N.Y. 11590, Biodesign International, 60 Industrial Park Road, Saco, Me. 04072, and Research Diagnostics Inc., Pleasant Hill Road, Flanders N.J. 07836.

Preferred LRPI antibodies are set forth in Table XXV or are any antibody specific to the following proteins: gelsolin, factor XII, transthretin precursor, (pre-albumin), complement C3 precursor, alpha-1-macroglobulin, hemopexin precursor, paraoxonase/arylesterase 1, vitamin D-binding protein precursor, contrapsin-like protease inhibitor 3 precursor, contrapsin-like protease inhibitor, apolipoprotein E precursor, transferrin precursor, s-adenosylmethionine synthetase gamma form (methionine adenosyltransferase), apolipoprotein A1 precursor, malate dehydrogenase, purine nucleoside phosphorylase, glutathione peroxidase, beta-2-glycoprotein I precursor, 4-hydroxyphenylpyruvate dioxygenase, thrombospondin I precursor, plasma retinol-binding protein precursor, retinal binding protein 1.

Table XXV. Known Antibodies That Recognize LRPIs or LRPI-Related Polypeptides

| | | | | Catalogue |
|---|---|---|---|---|
| LRF# | LRPI# | Antibody | Manufacturer | Number |
| LRF-36 | LRPI-36.1 | Albumin, Human, | ACCURATE | IMS-01-026-02 |
| | | Chicken anti- | CHEMICAL & | |
| | | | SCIENTIFIC | |
| | | | CORPORATION | |
| LRF-132 | LRPI-132.2 | Albumin, Human, | ACCURATE | IMS-01-026-02 |
| | | Chicken anti- | CHEMICAL & | |
| | | | SCIENTIFIC | |
| | | | CORPORATION | |
| LRF-182 | LRPI-182.1 | Albumin, Human, | ACCURATE | IMS-01-026-02 |
| | | Chicken anti- | CHEMICAL & | |
| | | | SCIENTIFIC | |
| | | | CORPORATION | |
| LRF-397 | LRPI-397.1 | Albumin, Human, | ACCURATE | IMS-01-026-02 |
| | | Chicken anti- | CHEMICAL & | |
| | | | SCIENTIFIC | |
| | | | CORPORATION | |
| LRF-399 | LRPI-399.1 | Albumin, Human, | ACCURATE | IMS-01-026-02 |
| | | Chicken anti- | CHEMICAL & | |
| | | | SCIENTIFIC | |
| | | | CORPORATION | |
| LRF-407 | LRPI-407.1 | Albumin, Human, | ACCURATE | IMS-01-026-02 |
| | | Chicken anti- | CHEMICAL & | |
| | | | SCIENTIFIC | |
| | | | CORPORATION | |
| LRF-24 | LRPI-24.1 | Anti-Cytokeratin Type 10 | RDI RESEARCH | RDI-CBL196 |
| | | | DIAGNOSTICS, INC | |
| LRF-115 | LRPI-115.1 | Anti-Cytokeratin Type 10 | RDI RESEARCH | RDI-CBL196 |
| | | | DIAGNOSTICS, INC | |
| LRF-123 | LRPI-123.2 | Anti-Cytokeratin Type 10 | RDI RESEARCH | RDI-CBL196 |
| | | | DIAGNOSTICS, INC | |
| LRF-394 | LRPI-394.2 | Anti-Cytokeratin Type 10 | RDI RESEARCH | RDI-CBL196 |
| | | | DIAGNOSTICS, INC | |
| LRF-8 | LRPI-8.1 | Apolipoprotein A1 (HDL), | ACCURATE | ACL-20075AP |
| | | Sheep anti-Human | CHEMICAL & | |
| | | | SCIENTIFIC | |
| | | | CORPORATION | |
| LRF-66 | LRPI-66.1 | Apolipoprotein A1 (HDL), | ACCURATE | ACL-20075AP |
| | | Sheep anti-Human | CHEMICAL & | |
| | | | SCIENTIFIC | |
| | | | CORPORATION | |
| LRF-111 | LRPI-111.1 | Apolipoprotein A1 (HDL), | ACCURATE | ACL-20075AP |
| | | Sheep anti-Human | CHEMICAL & | |
| | | | SCIENTIFIC | |
| | | | CORPORATION | |
| LRF-160 | LRPI-160.1 | Apolipoprotein A1 (HDL), | ACCURATE | ACL-20075AP |
| | | Sheep anti-Human | CHEMICAL & | |
| | | | SCIENTIFIC | |
| | | | CORPORATION | |
| LRF-169 | LRPI-169.1 | Apolipoprotein A1 (HDL), | ACCURATE | ACL-20075AP |
| | | Sheep anti-Human | CHEMICAL & | |
| | | | SCIENTIFIC | |
| | | | CORPORATION | |
| LRF-218 | LRPI-218.1 | Apolipoprotein A1 (HDL), | ACCURATE | ACL-20075AP |
| | | Sheep anti-Human | CHEMICAL & | |
| | | | SCIENTIFIC | |
| | | | CORPORATION | |
| LRF-259 | LRPI-259.1 | Apolipoprotein A1 (HDL), | ACCURATE | ACL-20075AP |
| | | Sheep anti-Human | CHEMICAL & | |
| | | | SCIENTIFIC | |
| | | | CORPORATION | |
| LRF-260 | LRPI-260.1 | Apolipoprotein A1 (HDL), | ACCURATE | ACL-20075AP |
| | | Sheep anti-Human | CHEMICAL & | |
| | | | SCIENTIFIC | |
| | | | CORPORATION | |
| LRF-261 | LRPI-261.1 | Apolipoprotein A1 (HDL), | ACCURATE | ACL-20075AP |
| | | Sheep anti-Human | CHEMICAL & | |
| | | | SCIENTIFIC | |
| | | | CORPORATION | |
| LRF-387 | LRPI-387.1 | Apolipoprotein A1 (HDL), | ACCURATE | ACL-20075AP |
| | | Sheep anti-Human | CHEMICAL & | |
| | | | SCIENTIFIC | |
| | | | CORPORATION | |
| LRF-67 | LRPI-67.1 | Apolipoprotein E, LDL, | ACCURATE | YM-5029 |
| | | VLDL, Clone: 3D12, Mab | CHEMICAL & | |
| | | anti-Human, | SCIENTIFIC | |
| | | frozen/paraffin | CORPORATION | |
| LRF-161 | LRPI-161.1 | Apolipoprotein E, LDL, | ACCURATE | YM-5029 |
| | | VLDL, Clone: 3D12, Mab | CHEMICAL & | |
| | | anti-Human, | SCIENTIFIC | |
| | | frozen/paraffin | CORPORATION | |
| LRF-162 | LRPI-162.1 | Apolipoprotein E, LDL, | ACCURATE | YM-5029 |
| | | VLDL, Clone: 3D12, Mab | CHEMICAL & | |
| | | anti-Human, | SCIENTIFIC | |
| | | frozen/paraffin | CORPORATION | |
| LRF-163 | LRPI-163.1 | Apolipoprotein E, LDL, | ACCURATE | YM-5029 |
| | | VLDL, Clone: 3D12, Mab | CHEMICAL & | |
| | | anti-Human, | SCIENTIFIC | |
| | | frozen/paraffin | CORPORATION | |
| LRF-219 | LRPI-219.1 | Apolipoprotein E, LDL, | ACCURATE | YM-5029 |
| | | VLDL, Clone: 3D12, Mab | CHEMICAL & | |
| | | anti-Human, | SCIENTIFIC | |
| | | frozen/paraffin | CORPORATION | |
| LRF-262 | LRPI-262.1 | Apolipoprotein E, LDL, | ACCURATE | YM-5029 |
| | | VLDL, Clone: 3D12, Mab | CHEMICAL & | |
| | | anti-Human, | SCIENTIFIC | |
| | | frozen/paraffin | CORPORATION | |
| LRF-294 | LRPI-294.1 | Apolipoprotein E, LDL, | ACCURATE | YM-5029 |
| | | VLDL, Clone: 3D12, Mab | CHEMICAL & | |
| | | anti-Human, | SCIENTIFIC | |
| | | frozen/paraffin | CORPORATION | |
| LRF-359 | LRPI-359.1 | Apolipoprotein E, LDL, | ACCURATE | YM-5029 |
| | | VLDL, Clone: 3D12, Mab | CHEMICAL & | |
| | | anti-Human, | SCIENTIFIC | |
| | | frozen/paraffin | CORPORATION | |
| LRF-360 | LRPI-360.1 | Apolipoprotein E, LDL, | ACCURATE | YM-5029 |
| | | VLDL, Clone: 3D12, Mab | CHEMICAL & | |
| | | anti-Human, | SCIENTIFIC | |
| | | frozen/paraffin | CORPORATION | |
| LRF-361 | LRPI-361.1 | Apolipoprotein E, LDL, | ACCURATE | YM-5029 |
| | | VLDL, Clone: 3D12, Mab | CHEMICAL & | |
| | | anti-Human, | SCIENTIFIC | |
| | | frozen/paraffin | CORPORATION | |
| LRF-164 | LRPI-164.1 | C3 Complement, Chicken | ACCURATE | IMS-01-001-02 |
| | | anti-Human | CHEMICAL & | |
| | | | SCIENTIFIC | |
| | | | CORPORATION | |
| LRF-165 | LRPI-165.1 | C3 Complement, Chicken | ACCURATE | IMS-01-001-02 |
| | | anti-Human | CHEMICAL & | |
| | | | SCIENTIFIC | |
| | | | CORPORATION | |
| LRF-166 | LRPI-166.1 | C3 Complement, Chicken | ACCURATE | IMS-01-001-02 |
| | | anti-Human | CHEMICAL & | |
| | | | SCIENTIFIC | |
| | | | CORPORATION | |
| LRF-217 | LRPI-217.3 | C3 Complement, Chicken | ACCURATE | IMS-01-001-02 |
| | | anti-Human | CHEMICAL & | |
| | | | SCIENTIFIC | |
| | | | CORPORATION | |
| LRF-221 | LRPI-221.1 | C3 Complement, Chicken | ACCURATE | IMS-01-001-02 |
| | | anti-Human | CHEMICAL & | |
| | | | SCIENTIFIC | |
| | | | CORPORATION | |
| LRF-222 | LRPI-222.1 | C3 Complement, Chicken | ACCURATE | IMS-01-001-02 |
| | | anti-Human | CHEMICAL & | |
| | | | SCIENTIFIC | |
| | | | CORPORATION | |
| LRF-266 | LRPI-266.1 | C3 Complement, Chicken | ACCURATE | IMS-01-001-02 |
| | | anti-Human | CHEMICAL & | |
| | | | SCIENTIFIC | |
| | | | CORPORATION | |
| LRF-363 | LRPI-363.1 | C3 Complement, Chicken | ACCURATE | IMS-01-001-02 |
| | | anti-Human | CHEMICAL & | |
| | | | SCIENTIFIC | |
| | | | CORPORATION | |
| LRF-364 | LRPI-364.1 | C3 Complement, Chicken | ACCURATE | IMS-01-001-02 |
| | | anti-Human | CHEMICAL & | |
| | | | SCIENTIFIC | |
| | | | CORPORATION | |
| LRF-167 | LRPI-167.1 | C4 Complement, Chicken | ACCURATE | IMS-01-032-02 |
| | | anti-Human | CHEMICAL & | |
| | | | SCIENTIFIC | |
| | | | CORPORATION | |
| LRF-223 | LRPI-223.1 | C4 Complement, Chicken | ACCURATE | IMS-01-032-02 |
| | | anti-Human | CHEMICAL & | |
| | | | SCIENTIFIC | |
| | | | CORPORATION | |
| LRF-224 | LRPI-224.1 | C4 Complement, Chicken | ACCURATE | IMS-01-032-02 |
| | | anti-Human | CHEMICAL & | |
| | | | SCIENTIFIC | |
| | | | CORPORATION | |
| LRF-296 | LRPI-296.1 | C4 Complement, Chicken | ACCURATE | IMS-01-032-02 |
| | | anti-Human | CHEMICAL & | |
| | | | SCIENTIFIC | |
| | | | CORPORATION | |
| LRF-170 | LRPI-170.1 | Gelsolin, plasma + | ACCURATE | YBG-4628-6210 |
| | | cytoplasmic, Sheep anti- | CHEMICAL & | |
| | | | SCIENTIFIC | |
| | | | CORPORATION | |
| LRF-226 | LRPI-226.1 | Gelsolin, plasma + | ACCURATE | YBG-4628-6210 |
| | | cytoplasmic, Sheep anti- | CHEMICAL & | |
| | | | SCIENTIFIC | |
| | | | CORPORATION | |
| LRF-334 | LRPI-334.1 | Gelsolin, plasma + | ACCURATE | YBG-4628-6210 |
| | | cytoplasmic, Sheep anti- | CHEMICAL & | |
| | | | SCIENTIFIC | |
| | | | CORPORATION | |
| LRF-21 | LRPI-21.1 | Glyceraldehyde-3- | BIODESIGN | H86504M |
| | | Phosphate Dehydrogenase | INTERNATIONAL | |
| LRF-120 | LRPI-120.1 | Glyceraldehyde-3- | BIODESIGN | H86504M |
| | | Phosphate Dehydrogenase | INTERNATIONAL | |
| LRF-400 | LRPI-400.1 | Hemoglobin, Goat anti- | ACCURATE | BMD-J16 |
| | | Human | CHEMICAL & | |
| | | | SCIENTIFIC | |
| | | | CORPORATION | |
| LRF-172 | LRPI-172.1 | Hemopexin, Beta-1, | ACCURATE | YN-RHHPX |
| | | Rabbit anti-Human, | CHEMICAL & | |
| | | precipitating | SCIENTIFIC | |
| | | | CORPORATION | |
| LRF-173 | LRPI-173.1 | Hemopexin, Beta-1, | ACCURATE | YN-RHHPX |
| | | Rabbit anti-Human, | CHEMICAL & | |
| | | precipitating | SCIENTIFIC | |
| | | | CORPORATION | |
| LRF-174 | LRPI-174.1 | Hemopexin, Beta-1, | ACCURATE | YN-RHHPX |
| | | Rabbit anti-Human, | CHEMICAL & | |
| | | precipitating | SCIENTIFIC | |
| | | | CORPORATION | |
| LRF-227 | LRPI-227.1 | Hemopexin, Beta-1, | ACCURATE | YN-RHHPX |
| | | Rabbit anti-Human, | CHEMICAL & | |
| | | precipitating | SCIENTIFIC | |
| | | | CORPORATION | |
| LRF-228 | LRPI-228.1 | Hemopexin, Beta-1, | ACCURATE | YN-RHHPX |
| | | Rabbit anti-Human, | CHEMICAL & | |
| | | precipitating | SCIENTIFIC | |
| | | | CORPORATION | |
| LRF-335 | LRPI-335.1 | Hemopexin, Beta-1, | ACCURATE | YN-RHHPX |
| | | Rabbit anti-Human, | CHEMICAL & | |
| | | precipitating | SCIENTIFIC | |
| | | | CORPORATION | |
| LRF-365 | LRPI-365.1 | Hemopexin, Beta-1, | ACCURATE | YN-RHHPX |
| | | Rabbit anti-Human, | CHEMICAL & | |
| | | precipitating | SCIENTIFIC | |
| | | | CORPORATION | |
| LRF-366 | LRPI-366.1 | Hemopexin, Beta-1, | ACCURATE | YN-RHHPX |
| | | Rabbit anti-Human, | CHEMICAL & | |
| | | precipitating | SCIENTIFIC | |
| | | | CORPORATION | |
| LRF-367 | LRPI-367.1 | Hemopexin, Beta-1, | ACCURATE | YN-RHHPX |
| | | Rabbit anti-Human, | CHEMICAL & | |
| | | precipitating | SCIENTIFIC | |
| | | | CORPORATION | |
| LRF-178 | LRPI-178.1 | Monoclonal mouse anti- | RDI RESEARCH | RDI-TRK4P11- |
| | | human plasminogen | DIAGNOSTICS, INC | 4D2 |
| LRF-179 | LRPI-179.1 | Monoclonal mouse anti- | RDI RESEARCH | RDI-TRK4P11- |
| | | human plasminogen | DIAGNOSTICS, INC | 4D2 |
| LRF-14 | LRPI-14.1 | Monoclonal to Vimentin, | Abcam Ltd | ab66 |
| | | intermediate filaments | | |
| LRF-38 | LRPI-38.1 | Monoclonal to Vimentin, | Abcam Ltd | ab66 |
| | | intermediate filaments | | |
| LRF-168 | LRPI-168.1 | Mouse anti-human | RDI RESEARCH | RDI- |
| | | complement component | DIAGNOSTICS, INC | COMPC9abm-A6 |
| | | C9 (monoclonal) | | |
| LRF-84 | LRPI-84.1 | Polyclonal Rabbit anti- | RDI RESEARCH | RDI-CYTOK1abr |
| | | Human Cytokeratin 1 | DIAGNOSTICS, INC | |
| | | (Keratin 1) | | |
| LRF-123 | LRPI-123.1 | Polyclonal Rabbit anti- | RDI RESEARCH | RDI-CYTOK1abr |
| | | Human Cytokeratin 1 | DIAGNOSTICS, INC | |
| | | (Keratin 1) | | |
| LRF-300 | LRPI-300.1 | Prothrombin, Rabbit anti- | ACCURATE | AXL-448/2 |
| | | Human | CHEMICAL & | |
| | | | SCIENTIFIC | |
| | | | CORPORATION | |
| LRF-12 | LRPI-12.1 | Rabbit anti-Catalase | Abcam Ltd. | ab6572 |
| | | (polyclonal) | | |
| LRF-177 | LRPI-177.1 | Retinol Binding Protein, | ACCURATE | AXL-163/2 |
| | | Rabbit anti-Human | CHEMICAL & | |
| | | | SCIENTIFIC | |
| | | | CORPORATION | |
| LRF-236 | LRPI-236.1 | Transthyretin, Prealbumin, | ACCURATE | MED-CLA 193 |
| | | 55 kD, Rabbit anti-Human | CHEMICAL & | |
| | | | SCIENTIFIC | |
| | | | CORPORATION | |
| LRF-373 | LRPI-373.1 | Transthyretin, Prealbumin, | ACCURATE | MED-CLA 193 |
| | | 55 kD, Rabbit anti-Human | CHEMICAL & | |
| | | | SCIENTIFIC | |
| | | | CORPORATION | |

In one embodiment, binding of antibody in tissue sections can be used to detect LRPI localization or level of one or more LRPIs, for example, to identify substantial changes that may suggest abnormality. In a specific embodiment, antibody to an LRPI can be used to assay a tissue sample (e.g., a liver biopsy) for the level of the LRPI where a substantially changed level of LRPI is indicative of the presence of liver response. As used herein, a “substantially changed level” means a level that is increased or decreased relative to or compared with the level in a subject free from liver response, or that is compared with a reference level. Whether the changed level is substantial or significant is determined by assessing the relative abundance in sample versus control and whether the difference in levels is significant as assessed by analysis of p value. Particularly preferred are p values that are less than about 0.05 and relative abundance that is a positive or negative integer greater than about 1.0, particularly greater than about 1.2. If desired, the comparison can be performed with a matched sample from the same subject, taken from a portion of the body not affected by a liver response.

Any suitable immunoassay can be used to detect an LPRI, including, without limitation, competitive and non-competitive assay systems using techniques such as western blots, radioimmunoassays, ELISAs (enzyme linked immunosorbent assays), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

For example, an LRPI can be detected in a fluid sample (e.g., spinal fluid, blood, urine, or tissue homogenate) by means of a two-step sandwich assay. In the first step, a capture reagent (e.g., an anti-LRPI antibody) is used to capture the LRPI. Examples of such antibodies known in the art are set forth in Table XXV. The capture reagent can optionally be immobilized on a solid phase. In the second step, a directly- or indirectly-labeled detection reagent is used to detect the captured LRPI. In one embodiment, the detection reagent is a lectin. A lectin can be used for this purpose that preferentially binds to the LRPI rather than to other isoforms that have the same core protein as the LRPI or to other proteins that share the antigenic determinant recognized by the antibody. In a preferred embodiment, the chosen lectin binds to the LRPI with at least 2-fold greater affinity, more preferably at least 5-fold greater affinity, still more preferably at least 10-fold greater affinity, than to said other isoforms that have the same core protein as the LRPI or to said other proteins that share the antigenic determinant recognized by the antibody. Based on the present description, a lectin that is suitable for detecting a given LRPI can readily be identified by those skilled in the art, by methods well known in the art, for instance upon testing one or more lectins enumerated in Table I on pages 158-159 of Sumar et al., Lectins as Indicators of Disease-Associated Glycoforms, In: Gabius H-J & Gabius S (eds.), 1993, Lectins and Glycobiology, at pp. 158-174. Lectins with the desired oligosaccharide specificity can be identified, for example, by their ability to detect the LRPI in a 2D gel, in a replica of a 2D gel following transfer to a suitable solid substrate such as a nitrocellulose membrane, or in a two-step assay following capture by an antibody. In an alternative embodiment, the detection reagent is an antibody, e.g., an antibody that immunospecifically detects other post-translational modifications, such as an antibody that immunospecifically binds to phosphorylated amino acids. Examples of such antibodies include those that bind to phosphotyrosine (BD Transduction Laboratories, catalog nos.: P11230-050/P11230-150; P11120; P38820; P39020), those that bind to phosphoserine (Zymed Laboratories Inc., South San Francisco, Calif., catalog no. 61-8100) and those that bind to phosphothreonine (Zymed Laboratories Inc., South San Francisco, Calif., catalog nos. 71-8200, 13-9200).

If desired, a gene encoding an LRPI, a related gene (e.g. a gene having sequence homology), or related nucleic acid sequences or subsequences, including complementary sequences, can also be used in hybridization assays. A nucleotide encoding an LRPI, or subsequences thereof comprising at least 8 nucleotides, preferably at least 12 nucleotides, and most preferably at least 15 nucleotides can be used as a hybridization probe. Hybridization assays can be used for detection, treatment, prognosis, diagnosis, or monitoring of liver responseassociated with aberrant expression of genes encoding LRPIs, or for differential diagnosis of subjects with signs or symptoms suggestive of a liver response. In particular, such a hybridization assay can be carried out by a method comprising contacting a subject's sample containing nucleic acid with a nucleic acid probe capable of hybridizing to a DNA or RNA that encodes an LRPI, under conditions such that hybridization can occur, and detecting or measuring any resulting hybridization. Nucleotides can be used for therapy of subjects having liver response, as described below.

The invention also provides diagnostic kits, comprising an anti-LRPI antibody. In addition, such a kit may optionally comprise one or more of the following: (1) instructions for using the anti-LRPI antibody for diagnosis, prognosis, therapeutic monitoring or any combination of these applications; (2) a labeled binding partner to the antibody; (3) a solid phase (such as a reagent strip) upon which the anti-LRPI antibody is immobilized; and (4) a label or insert indicating regulatory approval for diagnostic, prognostic or therapeutic use or any combination thereof. If no labeled binding partner to the antibody is provided, the anti-LRPI antibody itself can be labeled with a detectable marker, e.g., a chemiluminescent, enzymatic, fluorescent, or radioactive moiety.

The invention also provides a kit comprising a nucleic acid probe capable of hybridizing to RNA encoding an LRPI. In a specific embodiment, a kit comprises in one or more containers a pair of primers (e.g., each in the size range of 6-30 nucleotides, more preferably 10-30 nucleotides and still more preferably 10-20 nucleotides) that under appropriate reaction conditions can prime amplification of at least a portion of a nucleic acid encoding an LRPI, such as by polymerase chain reaction (see, e.g., Innis et al., 1990, PCR Protocols, Academic Press, Inc., San Diego, Calif.), ligase chain reaction (see EP 320,308) use of Qβ replicase, cyclic probe reaction, or other methods known in the art and/or based on the present description.

Kits are also provided which allow for the detection of a plurality of LRPIs or a plurality of nucleic acids each encoding an LRPI. A kit can optionally further comprise a predetermined amount of an isolated LRPI protein or a nucleic acid encoding an LRPI, e.g., for use as a standard or control.

Statistical Techniques for Identifying LRFs, LRPIs and LRPI Clusters

Univariate differential analysis tools, such as fold changes, Wilcoxon rank sum test and t-test, are useful in identifying individual LRFs or LRPIs that are diagnostically associated with liver response or in identifying individual LRPIs that regulate the disease process. However, those skilled in the art will appreciate that a liver response is associated with a combination of LRFs or LRPIs (and to be regulated by a suitable combination of LRPIs), rather than individual LRFs and LRPIs in isolation. The strategies for discovering such suitable combinations of LRFs and LRPIs differ from those for discovering individual LRFs and LRPIs. In such cases, each individual LRF and LRPI can be regarded as one variable and the disease can be regarded as a joint, multivariate effect caused by interaction of these variables.

The following steps can be used to identify markers from data produced in accordance herewith, as by use of the Preferred Technology.

The first step is to identify a collection of LRFs or LRPIs that individually show a significant association with the presence of liver response. The association between the identified individual LRFs or individual LRPIs and liver response need not be as highly significant when a collection of LRFs or LRPIs is used, as is desirable when an individual LRF or LRPI is used as a diagnostic. Any of the tests discussed above (fold changes, Wilcoxon rank sum test, etc.) can be used at this stage. Once a suitable collection of LRFs or LRPIs has been identified, a sophisticated multi-variate analysis capable of identifying clusters can then be used to estimate the significant multivariate associations with liver response.

Linear Discriminant Analysis (LDA) is one such procedure, which can be used to detect significant association between a cluster of variables (i.e., LRFs or LRPIs) and liver response. In performing LDA, a set of weights is associated with each variable (i.e., LRF or LRPI) so that the linear combination of weights and the measured values of the variables can identify the presence of liver response by discriminating between subjects with liver response and subjects free from liver response. Enhancements to the LDA allow stepwise inclusion (or removal) of variables to optimize the discriminant power of the model. The result of the LDA is a cluster of LRFs or LRPIs which can be used for diagnosis, prognosis, therapy or development of pharmaceutical products. Other enhanced variations of LDA, such as Flexible Discriminant Analysis permit the use of non-linear combinations of variables to discriminate a liver response state from a normal state in which there is no liver response. The results of the discriminant analysis can be verified by post-hoc tests and also by repeating the analysis using alternative techniques such as classification trees.

A further category of LRFs or LRPIs can be identified by qualitative measures by comparing the percentage feature presence of an LRF or LRPI of one group of samples (e.g., samples from diseased subjects) with the percentage feature presence of an LRF or LRPI in another group of samples (e.g., samples from control subjects). The “percentage feature presence” of an LRF or LRPI is the percentage of samples in a group of samples in which the LRF or LRPI is detectable by the detection method of choice. For example, if an LRF is detectable in 95 percent of samples from diseased subjects, the percentage feature presence of that LRF in that sample group is 95 percent. If only 5 percent of samples from subjects not showing a liver response have detectable levels of the same LRF, detection of that LRF in the sample of a subject would suggest that it is likely that the subject has a liver response.

Use in Clinical Studies

The diagnostic methods and compositions of the present invention can assist in monitoring a clinical study, e.g. to evaluate potential active agents for their tendency to cause liver response in a patient or patient cohort. In one embodiment, potential active agents, etc., are tested for their ability to cause liver response by comparing the levels of LRFs or LRPI in a subject having the treatment to levels found in subjects free from liver response or, in a untreated subject (e.g. a subject receiving a placebo therapy). Alterations in the levels of one or more LRFs or LRPIs known to be significantly altered in patients who exhibit liver response indicate the presence of a liver response to that agent.

In another embodiment, the methods and compositions of the present invention are used to screen individuals for entry into a clinical study to identify individuals prone to developing liver response; individuals already showing a liver response, or who are likely to have a liver response after treatment can then be excluded from the study or can be placed in a separate cohort for treatment or analysis. If desired, the candidates can concurrently be screened to identify individuals with elevated liver enzymes, for example alanine aminotransferase and/or aspartate aminotransferase levels, or other known measures of liver response; procedures for these screens are well known in the art and include routine clinical chemistry assessments (see “The Clinical Chemistry of Laboratory Animals” (Pergamon Press), ed. Walter F. Loeb and Fred W. Quimby).

In a further embodiment, the methods and compositions of the present invention are used to evaluate patients and select the appropriate cohort or group(s) of patients for clinical assessment or for evaluation in a clinical trial. The methods and compositions of the present invention may be utilized to identify patients most at risk for or having liver response.

The invention provides a method for predicting the occurrence of liver response comprising:

a) exposing a target cell, tissue or biological organism to the environmental factor (e.g. candidate agent_;

b) obtaining a test sample from the target;

c) detecting and/or quantifying in the sample an LRPI or a plurality of LRPIs; and

d) comparing the abundance of the LRPI(s) in the test sample with the abundance of said LRPI(s) in a control sample, or with a previously determined reference range for said polypeptide in targets free from liver response, or with the abundance at least one standard polypeptide in the test sample.

The invention also provides a method for predicting the ability of an environmental factor (e.g. a candidate agent) to cause liver response comprising:

a) providing a plurality of identical spatial arrays antibodies comprising at least one antibody that specifically binds to one or more LRPIs;

b) exposing a biological sample or biological organism to the environmental factor;

c) extracting and isolating a polypeptide containing mixture from said exposed liver target of step b);

d) extracting and isolating a control polypeptide containing mixture from an equivalent biological sample or biological organism not exposed to said environmental factor of step b);

e) exposing a spatial array as defined in a) to the mixture extracted in b), and detecting the binding of polypeptides to the antibodies;

f) exposing a spatial array as defined in a) to the mixture extracted in c) and detecting the binding of polypeptides to the antibodies;

g) comparing the first and second binding patterns detected in step e) and f) to identify any change in said test pattern from the control pattern, indicative of the toxic effect of the test compound.

The diagnostic arrays of the invention may optionally be combined with one or more additional antibodies to known biomarkers for liver response.

Based on the methods outlined above, the polypeptide mixture that is isolated from the target is determined by the type of biological sample. Wherein, if the target is isolated liver cells or tissue, then the preferred isolated polypeptide mixture for use in the method can be for example, the supernatant. If the target is the liver organ in a mammalian subject, the isolated polypeptide mixture is preferably a body fluid, such as but not limited to blood, serum, plasma or urine. More preferably, the polypeptide mixture that is isolated is blood.

In a preferred embodiment, environmental factors that modulate (i.e., upregulate or downregulate) the expression, activity or both the expression and activity of a polypeptide of the invention are identified in a mammalian subject e.g. an animal model. Examples of suitable animals include, but are not limited to, mice, rats, rabbits, monkeys, guinea pigs, dogs and cats. A preferred animal model is the rodent, e.g. a rat. In accordance with this embodiment, the environmental factor or a control agent is administered (e.g., orally, rectally or parenterally such as intraperitoneally or intravenously) to a suitable animal and the effect on the expression, activity or both expression and activity of the polypeptide is determined. Changes in the expression of a polypeptide can be assessed by any suitable method described above, based on the present description.

Purification Of LRPIs

In particular aspects, the invention provides isolated LRPIs, that may be of mammalian or human origin, and fragments thereof which comprise an antigenic determinant (i.e., can be recognized by an antibody) or which are otherwise functionally active, as well as nucleic acid sequences encoding the foregoing. “Functionally active” as used herein refers to material displaying one or more functional activities associated with a full-length LRPI, e.g., binding to an LRPI substrate or LRPI binding partner, antigenicity (binding to an anti-target antibody), immunogenicity, enzymatic activity and the like.

In specific embodiments, the invention provides fragments of an LRPI comprising at least 5 amino acids, at least 10 amino acids, at least 50 amino acids, or at least 75 amino acids. Fragments lacking some or all of the regions of an LRPI are also provided, as are proteins (e.g., fusion proteins) comprising such fragments. Nucleic acids encoding the foregoing are provided.

Once a recombinant nucleic acid which encodes the LRPI, a portion of the LRPI, or a precursor of the LRPI is identified, the gene product can be analyzed. This can be achieved by assays based on the physical or functional properties of the given product, including for example, radioactive labeling of the product followed by analysis by gel electrophoresis, immunoassay, etc.

The LRPIs identified herein can be isolated and purified by standard methods including chromatography (e.g., ion exchange, affinity, and sizing column chromatography), centrifugation, differential solubility, or by any other standard technique for the purification of proteins.

Alternatively, once a recombinant nucleic acid that encodes the LRPI is identified, the entire amino acid sequence of the LRPI can be deduced from the nucleotide sequence of the gene coding region contained in the recombinant nucleic acid. As a result, the protein can be synthesized by standard chemical methods known in the art (e.g., see Hunkapiller et al., 1984, Nature 310:105-111).

In another alternative embodiment, native LRPIs can be purified from subjects displaying a liver response, by standard methods such as those described above (e.g., immunoaffinity purification).

In a preferred embodiment, LRPIs are isolated by the Preferred Technology described supra. For preparative-scale runs, a narrow-range “zoom gel” having a pH range of 2 pH units or less is preferred for the isoelectric step, according to the method described in Westermeier, 1993, Electrophoresis in Practice (VCH, Weinheim, Germany), pp. 197-209; this modification permits a larger quantity of a target protein to be loaded onto the gel, and thereby increases the quantity of isolated LRPI that can be recovered from the gel. When used in this way for preparative-scale runs, the Preferred Technology typically provides up to 100 ng, and can provide up to 1 μg, of an isolated LRPI in a single run. Those of skill in the art will appreciate that a zoom gel can be used in any separation strategy which employs gel isoelectric focusing.

The invention thus provides an isolated LRPI, an isolated LRPI-related polypeptide, and an isolated derivative or fragment of an LRPI or an LRPI-related polypeptide; any of the foregoing can be produced by known recombinant DNA techniques or by chemical synthetic methods.

Isolation Of DNA Encoding An LRPI

Particular embodiments for the cloning of a gene encoding an LRPI, are presented below by way of example and not of limitation.

The nucleotide sequences of the present invention, including any DNA and RNA, and comprising a sequence encoding an LRPI or a fragment thereof, or an LRPI-related polypeptide, may be synthesized using methods known in the art, such as using conventional chemical approaches or polymerase chain reaction (PCR) amplification. The nucleotide sequences of the present invention also permit the identification and cloning of the gene encoding an LRPI homolog or LRPI ortholog including, for example, by screening cDNA libraries, genomic libraries or expression libraries.

For example, to clone a gene encoding an LRPI by PCR techniques, anchored degenerate oligonucleotides (or a set of most likely oligonucleotides) can be designed for all LRPI peptide fragments identified as part of the same protein. PCR reactions under a variety of conditions can be performed with relevant CDNA and genomic DNAs (e.g., from liver tissue or from cells of the immune system) from one or more species. Also vectorette reactions can be performed on any available CDNA and genomic DNA using the oligonucleotides (which preferably are nested) as above. Vectorette PCR is a method that enables the amplification of specific DNA fragments in situations where the sequence of only one primer is known. Thus, it extends the application of PCR to stretches of DNA where the sequence information is only available at one end. (Arnold C, 1991, PCR Methods Appl. 1(1):39-42; Dyer K D, Biotechniques, 1995, 19(4):550-2). Vectorette PCR may pe performed with probes that are, for example, anchored degenerate oligonucleotides (or most likely oligonucleotides) coding for LRPI peptide fragments, using as a template a genomic library or CDNA library pools.

Anchored degenerate oligonucleotides (and most likely oligonucleotides) can be designed for all LRPI peptide fragments. These oligonucleotides may be labelled and hybridized to filters containing CDNA and genomic DNA libraries. Oligonucleotides to different peptides from the same protein will often identify the same members of the library. The CDNA and genomic DNA libraries may be obtained from any suitable or desired mammalian species, for example from humans.

Nucleotide sequences comprising a nucleotide sequence encoding an LRPI or LRPI fragment of the present invention are useful, for example, for their ability to hybridize selectively with complementary stretches of genes encoding other proteins. Depending on the application, a variety of hybridization conditions may be employed to obtain nucleotide sequences at least about 30%, 35%, 40%, 45%, 50%, 55%, 60%, 65%, 70%, 75%, 80%, 85%, 90%, 95% or 99% identical, or 100% identical, to the sequence of a nucleotide encoding an LRPI.

For a high degree of selectivity, relatively stringent conditions are used to form the duplexes, such as low salt or high temperature conditions. As used herein, “highly stringent conditions” means hybridization to filter-bound DNA in 0.5 M NaHPO4, 7% sodium dodecyl sulfate (SDS), 1 mM EDTA at 65° C., and washing in 0.1× SSC/0.1% SDS at 68° C. (Ausubel F. M. et al., eds., 1989, Current Protocols in Molecular Biology, Vol. I, Green Publishing Associates, Inc., and John Wiley & Sons, Inc., New York, at p. 2.10.3; incorporated herein by reference in its entirety.) For some applications, less stringent conditions for duplex formation are required. As used herein “moderately stringent conditions” means washing in 0.2× SSC/0.1% SDS at 42° C. (Ausubel et al., 1989, supra). Hybridization conditions can also be rendered more stringent by the addition of increasing amounts of formamide, to destabilize the hybrid duplex. Thus, particular hybridization conditions can be readily manipulated, and will generally be chosen depending on the desired results. In general, convenient hybridization temperatures in the presence of 50% formamide are: 42° C. for a probe which is 95 to 100% identical to the fragment of a gene encoding an LRPI, 37° C. for 90 to 95% identity and 32° C. for 70 to 90% identity.

In the preparation of genomic libraries, DNA fragments are generated, some of which will encode parts or the whole of an LRPI. Any suitable method for preparing DNA fragments may be used in the present invention. For example, the DNA may be cleaved at specific sites using various restriction enzymes. Alternatively, one may use DNAse in the presence of manganese to fragment the DNA, or the DNA can be physically sheared, as for example, by sonication. The DNA fragments can then be separated according to size by standard techniques, including but not limited to agarose and polyacrylamide gel electrophoresis, column chromatography and sucrose gradient centrifugation. The DNA fragments can then be inserted into suitable vectors, including but not limited to plasmids, cosmids, bacteriophages lambda or T4, and yeast artificial chromosome (YAC). (See, e.g., Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. I, II; Ausubel F. M. et al., eds., 1989, Current Protocols in Molecular Biology, Vol. I, Green Publishing Associates, Inc., and John Wiley & sons, Inc., New York). The genomic library may be screened by nucleic acid hybridization to labeled probe (Benton and Davis, 1977, Science 196:180; Grunstein and Hogness, 1975, Proc. Natl. Acad. Sci. U.S.A. 72:3961).

Based on the present description, the genomic libraries may be screened with labeled degenerate oligonucleotide probes corresponding to the amino acid sequence of any peptide of the LRPI based on the present description, using optimal approaches well known in the art. Any probe used is at least 10 nucleotides, at least 15 nucleotides, at least 20 nucleotides, at least 25 nucleotides, at least 30 nucleotides, at least 40 nucleotides, at least 50 nucleotides, at least 60 nucleotides, at least 70 nucleotides, at least 80 nucleotides, or at least 100 nucleotides. Preferably a probe is 10 nucleotides or longer, and more preferably 15 nucleotides or longer.

As shown in Tables XIII to XXIV above, some LRPIs disclosed herein correspond to isoforms of previously identified proteins encoded by genes whose sequences are publicly known. To screen such a gene, any probe may be used that is complementary to the gene or its complement; preferably the probe is 10 nucleotides or longer, more preferably 15 nucleotides or longer.

The SWISS-PROT and trEMBL databases (held by the Swiss Institute of Bioinformatics (SIB) and the European Bioinformatics Institue (EBI) which are available at http://www.expasy.com/) and the GenBank database (held by the National Institute of Health (NIH) which is available at http://www.ncbi.nlm.nih.gov/GenBank/) provide protein sequences comprising the amino acid sequences listed for the LRPIs in Tables XIII to XXIV under the following accession numbers and each sequence is incorporated herein by reference. In many cases the protein sequences in the database will cross-reference a nucleic acid or gene sequence encoding the protein or related protein.

Table XXVI. Sequences encoding LRPIs, LRPI-Related Proteins or ERPIs

| | | Rat/Mouse* Accession | Human |
|---|---|---|---|
| LRF# | LRPI# | Numbers | Homologues |
| LRF-1 | LRPI-1.1 | P00507 | P00505 |
| LRF-2 | LRPI-2.1 | P05544 | — |
| LRF-3 | LRPI-3.1 | P04642 | P00338 |
| LRF-4 | LRPI-4.1 | O55239* | P40261 |
| LRF-6 | LRPI-6.1 | Q9WVK7 | Q16836 |
| LRF-8 | LRPI-8.1 | P04639 | P02647 |
| LRF-9 | LRPI-9.1 | P51863* | — |
| LRF-10 | LRPI-10.1 | P27653 | P11586 |
| LRF-11 | LRPI-11.1 | P07756 | P31327 |
| LRF-11 | LRPI-11.2 | P26043* | P35241 |
| LRF-11 | LRPI-11.3 | P21213 | P42357 |
| LRF-14 | LRPI-14.1 | P31000 | P08670 |
| LRF-15 | LRPI-15.1 | P97852 | P51659 |
| LRF-16 | LRPI-16.1 | P49890 | P49888 |
| LRF-17 | LRPI-17.1 | P49890 | P49888 |
| LRF-17 | LRPI-17.2 | P49889 | P49888 |
| LRF-18 | LRPI-18.1 | P19112 | P09467 |
| LRF-19 | LRPI-19.1 | P04041 | P07203 |
| LRF-21 | LRPI-21.1 | P04797 | P04406 |
| LRF-22 | LRPI-22.1 | 92362 | — |
| LRF-23 | LRPI-23.1 | P97519 | P35914 |
| LRF-24 | LRPI-24.1 | P06394 (bovine) | P13645 |
| LRF-25 | LRPI-25.1 | P19001* | P08727 |
| LRF-26 | LRPI-26.1 | 1334284 | P10809 |
| LRF-27 | LRPI-27.1 | 1800307 | — |
| LRF-28 | LRPI-28.1 | P04176 | P00439 |
| LRF-29 | LRPI-29.1 | P14882 | P05165 |
| LRF-30 | LRPI-30.1 | P04785 | P07237 |
| LRF-31 | LRPI-31.1 | 4105269 | 10190704 |
| LRF-32 | LRPI-32.1 | P52873 | P11498 |
| LRF-36 | LRPI-36.1 | P02770 | P02768 |
| LRF-37 | LRPI-37.1 | P24329 | Q16762 |
| LRF-38 | LRPI-38.1 | P31000 | P08670 |
| LRF-53 | LRPI-53.1 | P46953 | P46952 |
| LRF-54 | LRPI-54.1 | P10719 | P06576 |
| LRF-56 | LRPI-56.1 | P04182 | P04181 |
| LRF-56 | LRPI-56.2 | P04182 | P04181 |
| LRF-60 | LRPI-60.1 | P31210 | P51857 |
| LRF-61 | LRPI-61.1 | P07872 | Q15067 |
| LRF-64 | LRPI-64.1 | 205308 | — |
| LRF-66 | LRPI-66.1 | P04639 | P02647 |
| LRF-67 | LRPI-67.1 | P02650 | P02649 |
| LRF-69 | LRPI-69.1 | 91997 | 345752 |
| LRF-70 | LRPI-70.1 | 8048915 | — |
| LRF-71 | LRPI-71.1 | P14141 | P07451 |
| LRF-72 | LRPI-72.1 | P09006 | — |
| LRF-73 | LRPI-73.1 | — | 10934059 |
| LRF-74 | LRPI-74.1 | Q63342 | Q9UI17 |
| LRF-75 | LRPI-75.1 | P13803 | P13804 |
| LRF-75 | LRPI-75.2 | P52847 | — |
| LRF-76 | LRPI-76.1 | P02680 | P04469, P02679 |
| LRF-78 | LRPI-78.1 | O35077 | P21695 |
| LRF-79 | LRPI-79.1 | — | P17080 |
| LRF-79 | LRPI-79.2 | P20788 | P47985 |
| LRF-80 | LRPI-80.1 | Q61316* | P34932 |
| LRF-81 | LRPI-81.1 | — | O15142 |
| LRF-83 | LRPI-83.1 | 6014636* | 7657176 |
| LRF-84 | LRPI-84.1 | — | P04264 |
| LRF-85 | LRPI-85.1 | 8393730* | 5031871 |
| LRF-88 | LRPI-88.1 | P40112 | P49720 |
| LRF-89 | LRPI-89.1 | — | 7385135 |
| LRF-90 | LRPI-90.1 | P46462 | P55072 |
| LRF-91 | LRPI-91.1 | P32362 | P06132 |
| LRF-92 | LRPI-92.1 | — | 10434033 |
| LRF-107 | LRPI-107.1 | P00173 | P00167 |
| LRF-109 | LRPI-109.1 | P45953 | P49748 |
| LRF-110 | LRPI-110.1 | P25409 | P24298 |
| LRF-111 | LRPI-111.1 | P04639 | P02647 |
| LRF-112 | LRPI-112.1 | P10719 | P06576 |
| LRF-112 | LRPI-112.2 | Q63081 | Q15084 |
| LRF-113 | LRPI-113.1 | 3766201* | O95194 |
| LRF-114 | LRPI-114.1 | P07756 | P31327 |
| LRF-115 | LRPI-115.1 | — | P13645 |
| LRF-115 | LRPI-115.2 | P07756 | P31327 |
| LRF-119 | LRPI-119.1 | P50580* | Q9UQ80 |
| LRF-119 | LRPI-119.2 | P32020* | P22307 |
| LRF-119 | LRPI-119.3 | P10860 | P49448 |
| LRF-120 | LRPI-120.1 | P04797 | P04406 |
| LRF-121 | LRPI-121.1 | Q63060 | P32189 |
| LRF-123 | LRPI-123.1 | — | P04264 |
| LRF-123 | LRPI-123.2 | — | P13645 |
| LRF-124 | LRPI-124.1 | P27605 | P00492 |
| LRF-127 | LRPI-127.1 | 587518 | P05783 |
| LRF-130 | LRPI-130.1 | P12928 | P30613 |
| LRF-131 | LRPI-131.1 | P02696 | P09455 |
| LRF-132 | LRPI-132.1 | 3851614* | P31040 |
| LRF-132 | LRPI-132.2 | P02770 | P02768 |
| LRF-133 | LRPI-133.1 | — | 10800086 |
| LRF-156 | LRPI-156.1 | P17475 | P01010 |
| LRF-157 | LRPI-157.1 | 205384 | — |
| LRF-158 | LRPI-158.1 | 205384 | — |
| LRF-159 | LRPI-159.1 | 205384 | — |
| LRF-160 | LRPI-160.1 | P02651 | P06727 |
| LRF-161 | LRPI-161.1 | P02650 | P02649 |
| LRF-162 | LRPI-162.1 | P02650 | P02649 |
| LRF-163 | LRPI-163.1 | P02650 | P02649 |
| LRF-164 | LRPI-164.1 | P01026 | P01024 |
| LRF-165 | LRPI-165.1 | P01026 | P01024 |
| LRF-166 | LRPI-166.1 | P01026 | P01024 |
| LRF-167 | LRPI-167.1 | P01029* | P01028 |
| LRF-168 | LRPI-168.1 | Q62930 | P02748 |
| LRF-169 | LRPI-169.1 | P04639 | P02647 |
| LRF-170 | LRPI-170.1 | P13020* | P06396 |
| LRF-172 | LRPI-172.1 | P20059 | P02790 |
| LRF-173 | LRPI-173.1 | P20059 | P02790 |
| LRF-174 | LRPI-174.1 | P20059 | P02790 |
| LRF-175 | LRPI-175.1 | 111977 | — |
| LRF-175 | LRPI-175.2 | 111977 | — |
| LRF-176 | LRPI-176.1 | P55159 | P27169 |
| LRF-177 | LRPI-177.1 | P04916 | P02753 |
| LRF-178 | LRPI-178.1 | 5295890 | P00747 |
| LRF-179 | LRPI-179.1 | 5295890 | P00747 |
| LRF-181 | LRPI-181.1 | P18298 | P31153 |
| LRF-182 | LRPI-182.1 | P02770 | P02768 |
| LRF-183 | LRPI-183.1 | 90402* | — |
| LRF-184 | LRPI-184.1 | 198848 | P02787 |
| LRF-184 | LRPI-184.2 | P12346 | P02787 |
| LRF-217 | LRPI-217.1 | 809561 | P02570 |
| LRF-217 | LRPI-217.3 | P01026 | P01024 |
| LRF-218 | LRPI-218.1 | P04639 | P02647 |
| LRF-219 | LRPI-219.1 | P02650 | P02649 |
| LRF-220 | LRPI-220.1 | Q29545 (porcine) | — |
| LRF-221 | LRPI-221.1 | P01026 | P01024 |
| LRF-222 | LRPI-222.1 | P01026 | P01024 |
| LRF-223 | LRPI-223.1 | P01030 (bovine) | P01028 |
| LRF-224 | LRPI-224.1 | P01029* | P01028 |
| LRF-225 | LRPI-225.1 | 10946900* | P00748 |
| LRF-226 | LRPI-226.1 | P13020 | P06396 |
| LRF-227 | LRPI-227.1 | P20059 | P02790 |
| LRF-228 | LRPI-228.1 | P20059 | P02790 |
| LRF-230 | LRPI-230.1 | 7441758 | — |
| LRF-231 | LRPI-231.1 | 7441758 | — |
| LRF-232 | LRPI-232.1 | 220698 | — |
| LRF-232 | LRPI-232.2 | P05544 | — |
| LRF-234 | LRPI-234.1 | P35441* | P07996 |
| LRF-235 | LRPI-235.1 | P12346 | P02787 |
| LRF-236 | LRPI-236.1 | P02767 | P02766 |
| LRF-237 | LRPI-237.1 | P04276 | P02774 |
| LRF-256 | LRPI-256.1 | 205384 | — |
| LRF-257 | LRPI-257.1 | 205384 | — |
| LRF-258 | LRPI-258.1 | 205384 | — |
| LRF-259 | LRPI-259.1 | P04639 | P02647 |
| LRF-260 | LRPI-260.1 | P02651 | P06727 |
| LRF-261 | LRPI-261.1 | P02651 | P06727 |
| LRF-262 | LRPI-262.1 | P02650 | P02649 |
| LRF-263 | LRPI-263.1 | P26644 | P02749 |
| LRF-264 | LRPI-264.1 | P26644 | P02749 |
| LRF-265 | LRPI-265.1 | 5305687 | P08123 |
| LRF-266 | LRPI-266.1 | P01026 | P01024 |
| LRF-270 | LRPI-270.1 | P02761 | — |
| LRF-271 | LRPI-271.1 | P55159 | P27169 |
| LRF-294 | LRPI-294.1 | P02650 | P02649 |
| LRF-295 | LRPI-295.1 | P26644 | P02749 |
| LRF-296 | LRPI-296.1 | P01029* | P01028 |
| LRF-297 | LRPI-297.1 | 3435296 | P32754 |
| LRF-298 | LRPI-298.1 | P25093 | P16930 |
| LRF-300 | LRPI-300.1 | P18292 | P00734 |
| LRF-300 | LRPI-300.2 | 111977 | — |
| LRF-301 | LRPI-301.1 | 111977 | — |
| LRF-303 | LRPI-303.1 | 92471 | — |
| LRF-305 | LRPI-305.1 | P14152* | P40925 |
| LRF-306 | LRPI-306.1 | P23492* | P00491 |
| LRF-328 | LRPI-328.1 | 202857 | — |
| LRF-329 | LRPI-329.1 | 202857 | — |
| LRF-331 | LRPI-331.1 | 202857 | — |
| LRF-332 | LRPI-332.1 | P26644 | P02749 |
| LRF-334 | LRPI-334.1 | — | P06396 |
| LRF-335 | LRPI-335.1 | P20059 | P02790 |
| LRF-356 | LRPI-356.1 | — | P14314 |
| LRF-358 | LRPI-358.1 | 202857 | — |
| LRF-359 | LRPI-359.1 | P02650 | P02649 |
| LRF-360 | LRPI-360.1 | P02650 | P02649 |
| LRF-361 | LRPI-361.1 | P02650 | P02649 |
| LRF-363 | LRPI-363.1 | P01026 | P01024 |
| LRF-364 | LRPI-364.1 | P01026 | P01024 |
| LRF-365 | LRPI-365.1 | P20059 | P02790 |
| LRF-366 | LRPI-366.1 | P20059 | P02790 |
| LRF-367 | LRPI-367.1 | P20059 | P02790 |
| LRF-368 | LRPI-368.1 | 92471 | — |
| LRF-369 | LRPI-369.1 | 6689103 | 5459324 |
| LRF-370 | LRPI-370.1 | P55161 | Q9Y2A7 |
| LRF-371 | LRPI-371.1 | P05544 | — |
| LRF-372 | LRPI-372.1 | P12346 | P02787 |
| LRF-373 | LRPI-373.1 | P02767 | P02766 |
| LRF-386 | LRPI-386.1 | 6139055 | X66922 |
| LRF-387 | LRPI-387.1 | P04639 | P02647 |
| LRF-389 | LRPI-389.1 | P07872 | Q15067 |
| LRF-390 | LRPI-390.1 | O88267 | P49753 |
| LRF-391 | LRPI-391.1 | O09131* | P78417 |
| LRF-392 | LRPI-392.1 | 436935 | P25388 |
| LRF-393 | LRPI-393.1 | 6539658 | O14832 |
| LRF-394 | LRPI-394.1 | Q9QUM9* | P34062 |
| LRF-394 | LRPI-394.2 | — | P13645 |
| LRF-395 | LRPI-395.1 | P04904 | P08263 |
| LRF-395 | LRPI-395.2 | P23965 | P42126 |
| LRF-397 | LRPI-397.1 | P02770 | P02768 |
| LRF-399 | LRPI-399.1 | P02770 | P02768 |
| LRF-400 | LRPI-400.1 | P02091 | P02023 |
| LRF-400 | LRPI-400.2 | 130980 | P07737 |
| LRF-401 | LRPI-401.1 | P07824 | P05089 |
| LRF-402 | LRPI-402.1 | P10860 | P00367 |
| LRF-403 | LRPI-403.1 | P15999 | P25705 |
| LRF-405 | LRPI-405.1 | 5931565* | P10768 |
| LRF-405 | LRPI-405.2 | Q62651 | Q13011 |
| LRF-407 | LRPI-407.1 | P02770 | P02768 |
| LRF-409 | LRPI-409.1 | 6755212* | Q06323 |
| LRF-410 | LRPI-410.1 | O88569* | P22626 |
| LRF-12 | LRPI-12.1 | P04762 | P04040 |
| LRF-122 | LRPI-122.1 | P01946 | P01922 |
| LRF-126 | LRPI-126.1 | P38062 | P50579 |
| LRF-128 | LRPI-128.1 | Q10758 | P05787 |
| LRF-129 | LRPI-129.1 | 1334284 | P10809 |
| LRF-13 | LRPI-13.1 | P80254 | P30046 |
| LRF-134 | LRPI-134.1 | P28480 | P17987 |
| LRF-20 | LRPI-20.1 | O09131* | P78417 |
| LRF-63 | LRPI-63.1 | Q64640 | P55263 |
| LRF-77 | LRPI-77.1 | P14942 | P09210 |
| LRF-87 | LRPI-87.1 | 6755212* | Q06323 |

When no nucleotide sequence is known that encodes a protein comprising an amino acid sequence of a given LRPI, degenerate probes can be used for screening. In the method of tandem mass spectroscopy used for sequencing peptides in the present invention, the following pairs of amino acids could not be distinguished from each other: leucine and isoleucine; and, under certain circumstances, phenylalanine and oxidized methionine. As used herein, an amino acid sequence “as determined by mass spectrometry” refers to the set of amino acid sequences containing at the indicated positions, one or other member of the designated pairs of amino acids. For example, the amino acid sequence P[L/I]A indicates the amino acid sequences PLA and PIA. As will be obvious to one of skill in the art, a sequence containing n designated pairs indicates 2n amino acid sequences.

As used herein, the “mass of the singly protonated peptide” is the mass of the singly protonated tryptic digest peptide measured by mass spectrometry (having an error of measurement of approximately 100 parts-per-million or less) and corresponds to the total mass of the constituent amino acid residues of the peptide with the addition of a water molecule (H₂O) and a single proton (H⁺). As used herein, an “amino acid residue” refers to an amino acid residue of the general structure: —NH—CHR—CO— and which have the following symbols, elemental compositions and monoisotopic masses:

Amino Acid Residue Elemental Compositions and Monoisotopic Masses

| | | Elemental | Monoisotopic mass |
|---|---|---|---|
| Amino Acid | Symbol | Composition | (Da) |
| Alanine | A | C₃H₅NO | 71.037114 |
| Arginine | R | C₆H₁₂N₄O | 156.10111 |
| Asparagine | N | C₄H₆N₂O₂ | 114.042927 |
| Aspartic Acid | D | C₄H₅NO₃ | 115.026943 |
| Carboxyamido | C | C₅H₈N₂O₂S | 160.03065 |
| Cysteine¹ | | | |
| Glutamic Acid | E | C₅H₇NO₃ | 129.042593 |
| Glutamine | Q | C₃H₈N₂O₂ | 128.058577 |
| Glycine | G | C₂H₃NO | 57.021464 |
| Histadine | H | C₆H₇N₃O | 137.058912 |
| Isoleucine | I | C₆H₁₁NO | 113.084064 |
| Leucine | L | C₆H₁₁NO | 113.084064 |
| Lysine | K | C₆H₁₂N₂O | 128.094963 |
| Methionine | M | C₅H₉NOS | 131.040485 |
| Oxidised Methionine | M* | C₅H₉NO₂S | 147.035340 |
| Phenylalanine | F | C₉H₉NO | 147.068414 |
| Proline | P | C₅H₇NO | 97.052764 |
| Serine | S | C₃H₅NO₂ | 87.032028 |
| Threonine | T | C₄H₇NO₂ | 101.047678 |
| Tryptophan | W | C₁₁H₁₀N₂O | 186.079313 |
| Tyrosine | Y | C₉H₉NO₂ | 163.063328 |
| Valine | V | C₅H₉NO | 99.068414 |

¹All Cysteines are modified to the carboxyamino derivative during our production process.

As used herein “tryptic digest peptides” are peptides produced through treatment of the protein with the enzyme trypsin. Trypsin cleaves specifically at the carboxyl side of lysine (Lys) and arginine (Arg) residues, so that the tryptic digest peptides generated should have a Lys or Arg as the C-terminal amino acid, unless the peptide fragment was obtained from the C-terminal of the protein. Similarly, the amino acid directly preceding the N-terminal amino acid of the tryptic digest peptides should also be a Lys or Arg, unless the peptide was obtained from the N-terminal of the protein. The mass of a tryptic digest peptide corresponds to the total mass of the constituent amino acid residues of the peptide with the addition of a water molecule (H₂O). As used herein, the “partial sequence” is an amino acid sequence within the tryptic digest peptide determined from the interpretation of the tandem mass spectrum of the peptide. As used herein, the “N-terminal mass” is the mass measured by mass spectrometry (having an error of measurement of approximately 100 parts-per-million or less) of the portion of the tryptic digest peptide extending from the start of the partial sequence to the N-terminus of the peptide. This is a neutral mass corresponding to the total mass of the constituent amino acid residues extending from the partial sequence to the N-terminus of the peptide. As used herein, the “C-terminal mass” is the mass measured by mass spectrometry (having an error of measurement of approximately 100 parts-per-million or less) of the portion of the tryptic digest peptide extending from the end of the partial sequence to the C-terminus of the peptide. This mass corresponds to the total mass of the constituent amino acid residues extending from the end of the partial sequence to the C-terminus of the peptide with the addition of a water molecular (H₂O), and a single proton (H⁺).

Preferred and degenerate sets of probes may be described using GCG Nucleotide Ambiguity Codes as employed in GCG SeqWeb™ sequence analysis software (SeqWeb™ version 1.1, part of Wisconsin Package Version 10, Genetics Computer Group, Inc.). These Nucleotide Ambiguity Codes have the following meaning:

| GCG Code | Meaning |
|---|---|
| A | A |
| C | C |
| G | G |
| T | T |
| U | T |
| M | A or C |
| R | A or G |
| W | A or T |
| S | C or G |
| Y | C or T |
| K | G or T |
| V | A or C or G |
| H | A or C or T |
| D | A or G or T |
| B | C or G or T |
| X | G or A or T or C |
| N | G or A or T or C |

GCG uses the letter codes for amino acid codes and nucleotide ambiguity proposed by IUPAC-IUB. These codes are compatible with the codes used by the EMBL, GenBank, and PIR databases. See IUPAC, Commission on Nomenclature of Organic Chemistry. A Guide to IUPAC Nomenclature of Organic Compounds (Recommendations 1993), Blackwell Scientific publications, 1993.

When a library is screened, clones with insert DNA encoding the LRPI of interest, or a fragment thereof, will hybridize to one or more members of the corresponding set of degenerate oligonucleotide probes (or their complement). Hybridization of such oligonucleotide probes to genomic libraries may be carried out using methods known in the art. For example, hybridization with one of the above-mentioned degenerate sets of oligonucleotide probes, or their complement (or with any member of such a set, or its complement) can be performed under highly stringent or moderately stringent conditions as defined above, or can be carried out in 2× SSC, 1.0% SDS at 50° C. and washed using the washing conditions described supra for highly stringent or moderately stringent hybridization.

In yet another aspect of the invention, clones containing nucleotide sequences encoding the entire LRPI, a fragment of an LRPI, an LRPI-related polypeptide, or a fragment of an LRPI-related polypeptide any of the foregoing may also be obtained by screening expression libraries. For example, DNA from the relevant source is isolated and random fragments are prepared and ligated into an expression vector (e.g., a bacteriophage, plasmid, phagemid or cosmid) such that the inserted sequence in the vector is capable of being expressed by the host cell into which the vector is then introduced. Various screening assays can then be used to select for the expressed LRPI or LRPI-related polypeptides. In one embodiment, the various anti-LRPI antibodies of the invention can be used to identify the desired clones using methods known in the art. See, for example, Harlow and Lane, 1988, Antibodies: A Laboratory Manual, Cold Spring Harbor Laboratory Press, Cold Spring Harbor, N.Y., Appendix IV. Colonies or plaques from the library are brought into contact with the antibodies to identify those clones that bind antibody.

In an embodiment, colonies or plaques containing DNA that encodes an LRPI, a fragment of an LRPI, an LRPI-related polypeptide, or a fragment of an LRPI-related polypeptide can be detected using DYNA Beads according to Olsvick et al., 29th ICAAC, Houston, Tex. 1989, incorporated herein by reference. Anti-LRPI antibodies are crosslinked to tosylated DYNA Beads M280, and these antibody-containing beads are then contacted with colonies or plaques expressing recombinant polypeptides. Colonies or plaques expressing an LRPI or LRPI-related polypeptide are identified as any of those that bind the beads.

Alternatively, the anti-LRPI antibodies can be nonspecifically immobilized to a suitable support, such as silica or Celite® resin. This material is then used to adsorb to bacterial colonies expressing the LRPI protein or LRPI-related polypeptide as described herein.

In another aspect, PCR amplification may be used to isolate from genomic DNA a substantially pure DNA (i.e., a DNA substantially free of contaminating nucleic acids) encoding the entire LRPI or a part thereof. Preferably such a DNA is at least 95% pure, more preferably at least 99% pure. Oligonucleotide sequences, degenerate or otherwise, that correspond to peptide sequences of LRPIs disclosed herein can be used as primers.

PCR can be carried out, e.g., by use of a Perkin-Elmer Cetus thermal cycler and Taq polymerase (Gene Amp® or AmpliTaq DNA polymerase). One can choose to synthesize several different degenerate primers, for use in the PCR reactions. It is also possible to vary the stringency of hybridization conditions used in priming the PCR reactions, to allow for greater or lesser degrees of nucleotide sequence similarity between the degenerate primers and the corresponding sequences in the DNA. After successful amplification of a segment of the sequence encoding an LRPI, that segment may be molecularly cloned and sequenced, and utilized as a probe to isolate a complete genomic clone. This, in turn, will permit the determination of the gene's complete nucleotide sequence, the analysis of its expression, and the production of its protein product for functional analysis, as described infra.

The gene encoding an LRPI can also be identified by mRNA selection by nucleic acid hybridization followed by in vitro translation. In this procedure, fragments are used to isolate complementary mRNAs by hybridization. Such DNA fragments may represent available, purified DNA encoding an LRPI of another species (e.g., mouse, human). Immunoprecipitation analysis or functional assays (e.g., aggregation ability in vitro; binding to receptor) of the in vitro translation products of the isolated products of the isolated mRNAs identifies the mRNA and, therefore, the complementary DNA fragments that contain the desired sequences. In addition, specific mRNAs may be selected by adsorption of polysomes isolated from cells to immobilized antibodies that specifically recognize an LRPI. A radiolabelled cDNA encoding an LRPI can be synthesized using the selected mRNA (from the adsorbed polysomes) as a template. The radiolabelled mRNA or cDNA may then be used as a probe to identify the DNA fragments encoding an LRPI from among other genomic DNA fragments.

Alternatives to isolating genomic DNA encoding an LRPI include, but are not limited to, chemically synthesizing the gene sequence itself from a known sequence or making cDNA to the mRNA which encodes the LRPI. For example, RNA for cDNA cloning of the gene encoding an LRPI can be isolated from cells which express the LRPI. Those skilled in the art will understand from the present description that other methods may be used and are within the scope of the invention.

Any suitable eukaryotic cell can serve as the nucleic acid source for the molecular cloning of the gene encoding an LRPI. The nucleic acid sequences encoding the LRPI can be isolated from vertebrate, mammalian, primate, human, porcine, bovine, feline, avian, equine, canine or murine sources. The DNA may be obtained by standard procedures known in the art from cloned DNA (e.g., a DNA “library”), by chemical synthesis, by cDNA cloning, or by the cloning of genomic DNA, or fragments thereof, purified from the desired cell. (See, e.g., Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. I, II.) Clones derived from genomic DNA may contain regulatory and intron DNA regions in addition to coding regions; clones derived from cDNA will contain only exon sequences.

The identified and isolated gene or cDNA can then be inserted into any suitable cloning vector. A large number of vector-host systems known in the art may be used. As those skilled in the art will appreciate, the vector system chosen should be compatible with the host cell used. Such vectors include, but are not limited to, bacteriophages such as lambda derivatives, plasmids such as PBR322 or pUC plasmid derivatives or the Bluescript vector (Stratagene) or modified viruses such as adenoviruses, adeno-associated viruses or retroviruses. The insertion into a cloning vector can be accomplished, for example, by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and the gene encoding an LRPI may be modified by homopolymeric tailing. Recombinant molecules can be introduced into host cells via transformation, transfection, infection, electroporation, etc., so that many copies of the gene sequence are generated.

In specific embodiments, transformation of host cells with recombinant DNA molecules that incorporate the isolated gene encoding the LRPI, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

The nucleotide sequences of the present invention include nucleotide sequences encoding amino acid sequences with substantially the same amino acid sequences as native LRPIs, nucleotide sequences encoding amino acid sequences with functionally equivalent amino acids, nucleotide sequences encoding LRPIs, a fragments of LRPIs, LRPI-related polypeptides, or fragments of LRPI-related polypeptides.

In a specific embodiment, an isolated nucleic acid molecule encoding an LRPI-related polypeptide can be created by introducing one or more nucleotide substitutions, additions or deletions into the nucleotide sequence of an LRPI such that one or more amino acid substitutions, additions or deletions are introduced into the encoded protein. Standard techniques known to those of skill in the art can be used to introduce mutations, including, for example, site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

Expression of DNA Encoding LRPIs

The nucleotide sequence coding for an LRPI, an LRPI analog, an LRPI-related peptide, or a fragment or other derivative of any of the foregoing, can be inserted into an appropriate expression vector, i.e., a vector which contains the necessary elements for the transcription and translation of the inserted protein-coding sequence. The necessary transcriptional and translational signals can also be supplied by the native gene encoding the LRPI or its flanking regions, or the native gene encoding the LRPI-related polypeptide or its flanking regions. A variety of host-vector systems may be utilized in the present invention to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. In specific embodiments, a nucleotide sequence encoding a human gene (or a nucleotide sequence encoding a functionally active portion of a human LRPI) is expressed. In yet another embodiment, a fragment of an LRPI comprising a domain of the LRPI is expressed.

Any of the methods previously described for the insertion of DNA fragments into a vector may be used to construct expression vectors containing a chimeric gene consisting of appropriate transcriptional and translational control signals and the protein coding sequences. These methods may include in vitro recombinant DNA and synthetic techniques and in vivo recombinants (genetic recombination). Expression of nucleic acid sequence encoding an LRPI or fragment thereof may be regulated by a second nucleic acid sequence so that the LRPI or fragment is expressed in a host transformed with the recombinant DNA molecule. For example, expression of an LRPI may be controlled by any promoter or enhancer element known in the art. Promoters which may be used to control the expression of the gene encoding an LRPI or an LRPI-related polypeptide include, but are not limited to, the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), the tetracycline (Tet) promoter (Gossen et al., 1995, Proc. Nat. Acad. Sci. USA 89:5547-5551); prokaryotic expression vectors such as the β-lactamase promoter (Villa-Kumaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25; see also “Useful proteins from recombinant bacteria” in Scientific American, 1980, 242:74-94); plant expression vectors comprising the nopaline synthetase promoter region (Herrera-Estrella et al., Nature 303:209-213) or the cauliflower mosaic virus 35S RNA promoter (Gardner, et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94).

In a specific embodiment, a vector is used that comprises a promoter operably linked to an LRPI-encoding nucleic acid, one or more origins of replication, and, optionally, one or more selectable markers (e.g., an antibiotic resistance gene).

In a specific embodiment, an expression construct is made by subcloning an LRPI or an LRPI-related polypeptide coding sequence into the EcoRI restriction site of each of the three pGEX vectors (Glutathione S-Transferase expression vectors; Smith and Johnson, 1988, Gene 7:31-40). This allows for the expression of the LRPI product or LRPI-related polypeptide from the subclone in the correct reading frame.

In mammalian host cells, a number of viral-based expression systems may be utilized. In cases where an adenovirus is used as an expression vector, the LRPI coding sequence or LRPI-related polypeptide coding sequence may be ligated to an adenovirus transcription/translation control complex, e.g., the late promoter and tripartite leader sequence. This chimeric gene may then be inserted in the adenovirus genome by in vitro or if vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

Expression vectors containing inserts of a gene encoding an LRPI or an LRPI-related polypeptide can be identified, for example, by three general approaches: (a) nucleic acid hybridization, (b) presence or absence of “marker” gene functions, and (c) expression of inserted sequences. In the first approach, the presence of a gene encoding an LRPI inserted in an expression vector can be detected by nucleic acid hybridization using probes comprising sequences that are homologous to an inserted gene encoding an LRPI. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a gene encoding an LRPI in the vector. For example, if the gene encoding the LRPI is inserted within the marker gene sequence of the vector, recombinants containing the gene encoding the LRPI insert can be identified by the absence of the marker gene function. In the third approach, recombinant expression vectors can be identified by assaying the gene product (i.e., LRPI) expressed by the recombinant. Such assays can be based, for example, on the physical or functional properties of the LRPI in in vitro assay systems, e.g., binding with anti-LRPI antibody.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered LRPI or LRPI-related polypeptide may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system will produce an unglycosylated product and expression in yeast will produce a glycosylated product. Eukaryotic host cells which possess the cellular machinery for proper processing of the primary transcript, glycosylation, and phosphorylation of the gene product may be used. Such mammalian host cells include but are not limited to CHO, VERO, BHK, Hela, COS, MDCK, HEK293, 3T3, WI38, and in particular, hepatocyte cell lines such as, for example, HepG2, C3A, CRL-1439, NCTC and WRL-68. Furthermore, different vector/host expression systems may effect processing reactions to different extents.

For long-term, high-yield production of recombinant proteins, stable expression is preferred. For example, cell lines, which stably express the differentially expressed or pathway gene protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the differentially expressed or pathway gene protein. Such engineered cell lines may be particularly useful in screening and evaluation of compounds that affect the endogenous activity of the differentially expressed or pathway gene protein.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk-, hgprt- or aprt- cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes.

In other specific embodiments, the LRPI, fragment, analog, or derivative may be expressed as a fusion, or chimeric protein product (comprising the protein, fragment, analog, or derivative joined via a peptide bond to a heterologous protein sequence). For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification, increase half-life in vivo, and enhance the delivery of an antigen across an epithelial barrier to the immune system. An increase in the half-life in vivo and facilitated purification has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fe fragments (see, e.g., PCT publications WO 96/22024 and WO 99/04813).

Nucleic acids encoding an LRPI, a fragment of an LRPI, an LRPI-related polypeptide, or a fragment of an LRPI-related polypeptide can fused to an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897).

An LRPI fusion protein can be made by ligating the appropriate nucleic acid sequences encoding the desired amino acid sequences to each other by methods known in the art, in the proper coding frame, and expressing the chimeric product by methods commonly known in the art. Alternatively, an LRPI fusion protein may be made by protein synthetic techniques, e.g., by use of a peptide synthesizer.

Both cDNA and genomic sequences encoding these proteins can be cloned and expressed.

Domain Structure of LRPIs

Domains of some of the LRPIs provided by the present invention, are known in the art and have been described in the scientific literature. Moreover, domains of an LRPI can be identified using techniques known to those of skill in the art. For example, one or more domains of an LRPI can be identified by using one or more of the following programs: ProDom, TMpred, and SAPS. ProDom compares the amino acid sequence of a polypeptide to a database of compiled domains (see, e.g., http://www.toulouse.inra.fr/prodom.html; Corpet F., Gouzy J. & Kahn D., 1999, Nucleic Acids Res., 27:263-267). TMpred predicts membrane-spanning regions of a polypeptide and their orientation. This program uses an algorithm that is based on the statistical analysis of TMbase, a database of naturally occuring transmembrane proteins (see, e.g., http://www.ch.embnet.org/software/TMPRED™form.html; Hofmann & Stoffel. (1993) “TMbase—A database of membrane spanning proteins segments.” Biol. Chem. Hoppe-Seyler 347,166). The SAPS program analyzes polypeptides for statistically significant features like charge-clusters, repeats, hydrophobic regions, compositional domains (see, e.g., Brendel et al., 1992, Proc. Natl. Acad. Sci. USA 89: 2002-2006). Thus, based on the present description, those skilled in the art can identify domains of an LRPI having enzymatic or binding activity, and further can identify nucleotide sequences encoding such domains. These nucleotide sequences can then be used for recombinant expression of an LRPI fragment that retains the enzymatic or binding activity of the LRPI.

Based on the present description, those skilled in the art can identify domains of an LRPI having enzymatic or binding activity, and further can identify nucleotide sequences encoding such domains. These nucleotide sequences can then be used for recombinant expression of LRPI fragments that retain the enzymatic or binding activity of the LRPI.

In one embodiment, an LRPI has an amino acid sequence sufficiently similar to an identified domain of a known polypeptide. As used herein, the term “sufficiently similar” refers to a first amino acid or nucleotide sequence which contains a sufficient number of identical or equivalent (e.g., with a similar side chain) amino acid residues or nucleotides to a second amino acid or nucleotide sequence such that the first and second amino acid or nucleotide sequences have or encode a common structural domain or common functional activity or both.

An LRPI domain can be assessed for its function using techniques well known to those of skill in the art. For example, a domain can be assessed for its kinase activity or for its ability to bind to DNA using techniques known to the skilled artisan. Kinase activity can be assessed, for example, by measuring the ability of a polypeptide to phosphorylate a substrate. DNA binding activity can be assessed, for example, by measuring the ability of a polypeptide to bind to a DNA binding element in a electromobility shift assay.

Production of Antibodies to LRPIs

According to the invention an LRPI, LRPI analog, LRPI-related protein or a fragment or derivative of any of the foregoing may be used as an immunogen to generate antibodies which immunospecifically bind such an immunogen. Such immunogens can be isolated by any convenient means, including the methods described above. Antibodies of the invention include, but are not limited to polyclonal, monoclonal, bispecific, humanized or chimeric antibodies, single chain antibodies, Fab fragments and F(ab′) fragments, fragments produced by a Fab expression library, anti-idiotypic (anti-Id) antibodies, and epitope-binding fragments of any of the above. The term “antibody” as used herein refers to immunoglobulin molecules and immunologically active portions of immunoglobulin molecules, i.e., molecules that contain an antigen binding site that specifically binds an antigen. The immunoglobulin molecules of the invention can be of any class (e.g., IgG, IgE, IgM, IgD and IgA ) or subclass of immunoglobulin molecule.

In one embodiment, antibodies that recognize gene products of genes encoding LRPIs are publicly available. For example, antibodies that recognize these LRPIs and/or their isoforms include antibodies recognizing LRPI-1, LRPI-3, LRPI-4, LRPI-6, LRPI-7, LRPI-10, LRPI-15, LRPI-16, LRPI-22, LRPI-28, LRPI-30, LRPI-33, LRPI-34, LRPI-37, LRPI-38, LRPI-39, LRPI-40, LRPI-42, LRPI-43, LRPI-44, LRPI-45, LRPI-46, LRPI-47, LRPI-50, LRPI-52, LRPI-53, LRPI-55, LRPI-58, LRPI-60, LRPI-62, LRPI-64, LRPI-66, LRPI-67, LRPI-69, LRPI-72, LRPI-74, LRPI-75, LRPI-76, LRPI-77, LRPI-78, LRPI-79, LRPI-80, LRPI-81, LRPI-82, LRPI-84, LRPI-90, LRPI-92, LRPI-93, LRPI-95, LRPI-97, LRPI-98, LRPI-101, LRPI-102, LRPI-103, LRPI-104, LRPI-105, LRPI-110, LRPI-113, LRPI-115, LRPI-118, LRPI-119, LRPI-123, LRPI-124, LRPI-126, LRPI-130, LRPI-131, LRPI-132, LRPI-133, LRPI-134, LRPI-136, LRPI-137, LRPI-138, LRPI-140, LRPI-142, LRPI-143, LRPI-145, LRPI-149, LRPI-150, LRPI-161, LRPI-165, LRPI-167, LRPI-168, LRPI-169, LRPI-170, LRPI-171, LRPI-172, LRPI-173, LRPI-174, LRPI-175, LRPI-176, LRPI-178, LRPI-179, LRPI-181, LRPI-182, LRPI-186, LRPI-187, LRPI-188, LRPI-189 or LRPI-191. In another embodiment, methods known to those skilled in the art are used to produce antibodies that recognize an LRPI, an LRPI analog, an LRPI-related polypeptide, or a derivative or fragment of any of the foregoing.

In one embodiment of the invention, antibodies to a specific domain of an LRPI are produced. In a specific embodiment, hydrophilic fragments of an LRPI are used as immunogens for antibody production.

In the production of antibodies, screening for the desired antibody can be accomplished by techniques known in the art, e.g. ELISA (enzyme-linked immunosorbent assay). For example, to select antibodies which recognize a specific domain of an LRPI, one may assay generated hybridomas for a product which binds to an LRPI fragment containing such domain. For selection of an antibody that specifically binds a first LRPI homolog but which does not specifically bind to (or binds less avidly to) a second LRPI homolog, one can select on the basis of positive binding to the first LRPI homolog and a lack of binding to (or reduced binding to) the second LRPI homolog. Similarly, for selection of an antibody that specifically binds an LRPI but which does not specifically bind to (or binds less avidly to) a different isoform of the same protein (such as a different glycoform having the same core peptide as the LRPI), one can select on the basis of positive binding to the LRPI and a lack of binding to (or reduced binding to) the different isoform (e.g., a different glycoform). Thus, the present invention provides an antibody (preferably a monoclonal antibody) that binds with greater affinity (preferably at least 2-fold, more preferably at least 5-fold still more preferably at least 10-fold greater affinity) to an LRPI than to a different isoform or isoforms (e.g., glycoforms) of the LRPI.

Polyclonal antibodies which may be used in the methods of the invention are heterogeneous populations of antibody molecules derived from the sera of immunized animals. Unfractionated immune serum/plasma can also be used. Various procedures known in the art may be used for the production of polyclonal antibodies to an LRPI, a fragment of an LRPI, an LRPI-related polypeptide, or a fragment of an LRPI-related polypeptide. In a particular embodiment, rabbit polyclonal antibodies to an epitope of an LRPI or an LRPI-related polypeptide can be obtained. For example, for the production of polyclonal or monoclonal antibodies, various host animals can be immunized by injection with the native or a synthetic (e.g., recombinant) version of an LRPI, a fragment of an LRPI, an LRPI-related polypeptide, or a fragment of an LRPI-related polypeptide, including but not limited to rabbits, mice, rats, etc. The Preferred Technology described herein provides isolated LRPIs suitable for such immunization. If the LRPI is purified by gel electrophoresis, the LRPI can be used for immunization with or without prior extraction from the polyacrylamide gel. Various adjuvants may be used to enhance the immunological response, depending on the host species, including, but not limited to, complete or incomplete Freund's adjuvant, a mineral gel such as aluminum hydroxide, surface active substance such as lysolecithin, pluronic polyol, a polyanion, a peptide, an oil emulsion, keyhole limpet hemocyanin, dinitrophenol, and an adjuvant such as BCG (bacille Calmette-Guerin) or Corynebacterium parvum. Additional adjuvants are also well known in the art.

For preparation of monoclonal antibodies (mAbs) directed toward an LRPI, a fragment of an LRPI, an LRPI-related polypeptide, or a fragment of an LRPI-related polypeptide, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). Such antibodies may be of any immunoglobulin class including IgG, IgM, IgE, IgA, IgD and any subclass thereof. The hybridoma producing the mAbs of the invention may be cultivated in vitro or in vivo. In an additional embodiment of the invention, monoclonal antibodies can be produced in germ-free animals utilizing known technology (PCT/US90/02545, incorporated herein by reference).

The monoclonal antibodies include but are not limited to human monoclonal antibodies and chimeric monoclonal antibodies (e.g., human-mouse chimeras). Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.)

Chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, BioTechniques 4:214; U.S. Patent 5,225,539; Jones et al., 1986, Nature 321:552-525; Verhoeyan et al. (1988) Science 239:1534; and Beidler et al., 1988, J. Immunol. 141:4053-4060.

Completely human antibodies (antibodies derived solely from human antigenic material) are particularly desirable for therapeutic treatment of human subjects. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of an LRPI of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Fremont, Calif.) and Genpharm (San Jose, Calif.) can be engaged to provide human antibodies directed against selected antigens using technologies similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/Technology 12:899-903).

The antibodies of the present invention can also be generated using various phage display methods known in the art. In phage display methods, functional antibody domains are displayed on the surface of phage particles which carry the polynucleotide sequences encoding them. In a particular, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT Application No. PCT/GB91/01134; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of suitable techniques which can be used to produce single-chain Fvs and antibodies against LRPIs of the present invention, include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

The invention further provides for the use of bispecific antibodies, which can be made by methods known in the art. Traditional production of full length bispecific antibodies is based on the coexpression of two immunoglobulin heavy chain-light chain pairs, where the two chains have different specificities (Milstein et al., 1983, Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, published May 13, 1993, and in Traunecker et al., 1991, EMBO J. 10:3655-3659.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994. For further details for generating bispecific antibodies see, for example, Suresh et al., Methods in Enzymology,1986, 121:210.

The invention also provides functionally active fragments, derivatives or analogs of the anti-LRPI immunoglobulin molecules. Functionally active means that the fragment, derivative or analog is able to elicit anti-anti-idiotype antibodies (i.e., tertiary antibodies) that recognize the same antigen that is recognized by the antibody from which the fragment, derivative or analog is derived. Specifically, in a preferred embodiment the antigenicity of the idiotype of the immunoglobulin molecule may be enhanced by deletion of framework and CDR sequences that are C-terminal to the CDR sequence that specifically recognizes the antigen. To determine which CDR sequences bind the antigen, synthetic peptides containing the CDR sequences can be used with the antigen in any suitable binding assays known in the art.

The present invention provides antibody fragments such as, but not limited to, F(ab′)2 fragments and Fab fragments. Antibody fragments which recognize specific epitopes may be generated by known techniques. F(ab′)2 fragments consist of the variable region, the light chain constant region and the CH1 domain of the heavy chain and are generated by pepsin digestion of the antibody molecule. Fab fragments are generated by reducing the disulfide bridges of the F(ab′)2 fragments. The invention also provides heavy chain and light chain dimers of the antibodies of the invention, or any minimal fragment thereof such as Fvs or single chain antibodies (SCAs) (e.g., as described in U.S. Pat. No. 4,946,778; Bird, 1988, Science 242:423-42; Huston et al., 1988, Proc. Natl. Acad. Sci. USA 85:5879-5883; and Ward et al., 1989, Nature 334:544-54), or any other molecule with the same specificity as the antibody of the invention. Single chain antibodies are formed by linking the heavy and light chain fragments of the Fv region via an amino acid bridge, resulting in a single chain polypeptide. Techniques for the assembly of functional Fv fragments in E. coli may be used (Skerra et al., 1988, Science 242:1038-1041).

In other embodiments, the invention provides fusion proteins of the immunoglobulins of the invention (or functionally active fragments thereof), for example in which the immunoglobulin is fused via a covalent bond (e.g., a peptide bond), at either the N-terminus or the C-terminus to an amino acid sequence of another protein (or portion thereof, preferably at least 10, 20 or 50 amino acid portion of the protein) that is not the immunoglobulin. Preferably the immunoglobulin, or fragment thereof, is covalently linked to the other protein at the N-terminus of the constant domain. As stated above, such fusion proteins may facilitate purification, increase half-life in vivo, and enhance the delivery of an antigen across an epithelial barrier to the immune system.

The immunoglobulins of the invention include analogs and derivatives that are either modified, i.e, by the covalent attachment of any type of molecule as long as such covalent attachment that does not impair immunospecific binding. For example, but not by way of limitation, the derivatives and analogs of the immunoglobulins include those that have been further modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the analog or derivative may contain one or more non-classical or unnatural amino acids.

As those skilled in the art will appreciate based on the present description, the foregoing antibodies can be used in methods known in the art relating to the localization and activity of the LRPIs of the invention, e.g., for imaging these proteins, measuring levels thereof in appropriate physiological samples, in diagnostic methods, etc.

Expression Of Antibodies

The antibodies of the invention can be produced by any suitable method known in the art for the synthesis of antibodies, in particular, by chemical synthesis or by recombinant expression, and are preferably produced by recombinant expression technique.

Recombinant expression of antibodies, or fragments, derivatives or analogs thereof, requires construction of a nucleic acid that encodes the antibody. If the nucleotide sequence of the antibody is known, a nucleic acid encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding antibody, annealing and ligation of those oligonucleotides, and then amplification of the ligated oligonucleotides by PCR.

Alternatively, the nucleic acid encoding the antibody may be obtained by cloning the antibody. If a clone containing the nucleic acid encoding the particular antibody is not available, but the sequence of the antibody molecule is known, a nucleic acid encoding the antibody may be obtained from a suitable source (e.g., an antibody cDNA library, or cDNA library generated from any tissue or cells expressing the antibody) by PCR amplification using synthetic primers hybridizable to the 3′ and 5′ ends of the sequence or by cloning using an oligonucleotide probe specific for the particular gene sequence.

If an antibody molecule that specifically recognizes a particular antigen is not available (or a source for a cDNA library for cloning a nucleic acid encoding such an antibody), antibodies specific for a particular antigen may be generated by any method known in the art, for example, by immunizing an animal, such as a rabbit, to generate polyclonal antibodies or, more preferably, by generating monoclonal antibodies. Alternatively, a clone encoding at least the Fab portion of the antibody may be obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

Once a nucleic acid encoding at least the variable domain of the antibody molecule is obtained, it may be introduced into a vector containing the nucleotide sequence encoding the constant region of the antibody molecule (see, e.g., PCT Publication WO 86/05807; PCT Publication WO 89/01036; and U.S. Pat. No. 5,122,464). Vectors containing the complete light or heavy chain for co-expression with the nucleic acid to allow the expression of a complete antibody molecule are also available. Then, the nucleic acid encoding the antibody can be used to introduce the nucleotide substitution(s) or deletion(s) necessary to substitute (or delete) the one or more variable region cysteine residues participating in an intrachain disulfide bond with an amino acid residue that does not contain a sulfhydryl group. Such modifications can be carried out by any method known in the art for the introduction of specific mutations or deletions in a nucleotide sequence, for example, but not limited to, chemical mutagenesis, in vitro site directed mutagenesis (Hutchinson et al., 1978, J. Biol. Chem. 253:6551), PCT based methods, etc.

In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity can be used. As described and defined supra, a chimeric antibody is a molecule in which different portions are derived from different animal species, such as those having a variable region derived from a murine mAb and a human antibody constant region, e.g., humanized antibodies.

Once a nucleic acid encoding an antibody molecule of the invention has been obtained, the vector for the production of the antibody molecule may be produced by recombinant DNA technology using techniques well known in the art. Thus, methods for preparing the protein of the invention by expressing nucleic acid containing the antibody molecule sequences are described herein. Methods which are well known to those skilled in the art can be used to construct expression vectors containing an antibody molecule coding sequences and appropriate transcriptional and translational control signals. These methods include, for example, in vitro recombinant DNA techniques, synthetic techniques, and in vivo genetic recombination. See, for example, the techniques described in Sambrook et al. (1990, Molecular Cloning, A Laboratory Manual, 2d Ed., Cold Spring Harbor Laboratory, Cold Spring Harbor, N.Y.) and Ausubel et al. (eds., 1998, Current Protocols in Molecular Biology, John Wiley & Sons, NY).

The expression vector is transferred to a host cell by conventional techniques and the transfected cells are then cultured by conventional techniques to produce an antibody of the invention.

The host cells used to express a recombinant antibody of the invention may be either bacterial cells such as Escherichia coli, or, preferably, eukaryotic cells, especially for the expression of whole recombinant antibody molecule. In particular, mammalian cells such as Chinese hamster ovary cells (CHO), in conjunction with a vector such as the major intermediate early gene promoter element from human cytomegalovirus is an effective expression system for antibodies (Foecking et al., 198, Gene 45:101; Cockett et al., 1990, Bio/Technology 8:2).

A variety of host-expression vector systems may be utilized to express an antibody molecule of the invention. Such host-expression systems represent vehicles by which the coding sequences of interest may be produced and subsequently purified, but also represent cells which may, when transformed or transfected with the appropriate nucleotide coding sequences, express the antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing the antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the antibody molecule being expressed. For example, when a large quantity of such a protein is to be produced, for the generation of pharmaceutical compositions comprising an antibody molecule, vectors which direct the expression of high levels of fusion protein products that are readily purified may be desirable. Such vectors include, but are not limited, to the E. coli expression vector pUR278 (Ruther et al., 1983, EMBO J. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter). In mammalian host cells, a number of viral-based expression systems (e.g., an adenovirus expression system) may be utilized.

As discussed above, a host cell strain may be chosen based on the present description, which modulates the expression of the inserted sequences, or modifies and processes the gene product in the specific fashion desired. Such modifications (e.g., glycosylation) and processing (e.g., cleavage) of protein products may be important for the function of the protein.

For long-term, high-yield production of recombinant antibodies, stable expression is preferred. For example, cells lines that stably express an antibody of interest can be produced by transfecting the cells with an expression vector comprising the nucleotide sequence of the antibody and the nucleotide sequence of a selectable (e.g., neomycin or hygromycin), and selecting for expression of the selectable marker. Such engineered cell lines may be particularly useful in screening and evaluation of compounds that interact directly or indirectly with the antibody molecule.

The expression levels of the antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once the antibody molecule of the invention has been recombinantly expressed, it may be purified by any method known in the art for purification of an antibody molecule, for example, by chromatography (e.g., ion exchange chromatography, affinity chromatography such as with protein A or specific antigen, and sizing column chromatography), centrifugation, differential solubility, or by any other standard technique for the purification of proteins.

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with recombinant vaccinia virus are loaded onto Ni2+ nitriloacetic acid-agarose columns and histidine-tagged proteins are selectively eluted with imidazole-containing buffers.

Conjugated Antibodies

In a preferred embodiment, anti-LRPI antibodies or fragments thereof are conjugated to a diagnostic or a therapeutic molecule. The antibodies can be used for diagnosis or to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics according to the present invention. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include 1251, 1311, 111In and 99Tc.

An anti-LRPI antibodies or fragments thereof can be conjugated to a therapeutic agent or pharmaceutical product to modify a given biological response. The therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, a thrombotic agent or an anti-angiogenic agent, e.g., angiostatin or endostatin; or, a biological response modifier such as a lymphokine, interleukin-1 (IL-1), interleukin-2 (IL-2), interleukin-6 (IL-6), granulocyte macrophage colony stimulating factor (GM-CSF), granulocyte colony stimulating factor (G-CSF), nerve growth factor (NGF) or other growth factor.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described by Segal in U.S. Pat. No. 4,676,980. All of the foregoing citations are incorporated herein in their entireties.

An antibody with or without a therapeutic moiety conjugated to it can be used as a therapeutic that is administered alone or in combination with cytotoxic factor(s) and/or cytokine(s).

Diagnosis of Liver Response

In accordance with the present invention, suitable test samples, e.g. of blood, liver tissue, or urine obtained from an individual suspected of having or known to have liver response, as well as corresponding samples taken from normal individuals, can be used for diagnosis or monitoring. In one embodiment, a decreased abundance of one or more LRFs or LRPIs (or any suitable combination) in a test sample relative to a control sample (from a subject or subjects free from liver response) or a previously determined reference range indicates the presence of liver response; LRFs and LRPIs suitable for this purpose are identified in Tables I, III, V, VII, IX, XI, XIII, XV, XVII, XIX, XXI, and XXIII respectively, as described in detail above. In another embodiment of the invention, an increased abundance of one or more LRFs or LRPIs (or any suitable combination) in a test sample compared to a control sample or a previously determined reference range indicates the presence of liver response; LRFs and LRPIs suitable for this purpose are identified in Tables II, IV, VI, VIII, X, XII, XIV, XVI, XVIII, XX, XXII and XXIV, respectively, as described in detail above. In another embodiment, the relative abundance of one or more LRFs or LRPIs (or any suitable combination) in a test sample compared to a control sample or a previously determined reference range indicates a subtype of liver response (e.g.,). In yet another embodiment, the relative abundance of one or more LRFs or LRPIs (or any suitable combination) in a test sample relative to a control sample or a previously determined reference range indicates the degree or severity of liver response. In any of the aforesaid methods, detection of one or more LRPIs described herein may optionally be combined with detection of one or more additional biomarkers for liver response including, but not limited to 5′-nucleotidase, sorbitol dehydrogenase (SDH), alkaline phosphatase, ALT, AST, bilirubin, bile acids, albumin, total protein, gamma GT.

Any suitable method in the art can be employed to measure the level of LRFs and LRPIs, including but not limited to the Preferred Technology described herein, kinase assays, immunoassays to detect and/or visualize the LRPI (e.g., Western blot, immunoprecipitation followed by sodium dodecyl sulfate polyacrylamide gel electrophoresis, immunocytochemistry, etc.). In cases where an LRPI has a known function, an assay for that function may be used to measure LRPI expression. In a further embodiment, a decreased abundance of mRNA including one or more LRPIs identified in Table XIII, XV, XVII, XIX, XXI and XXIII, (or any suitable combination) in a test sample relative to a control sample or a previously determined reference range indicates the presence of liver response. In yet a further embodiment, an increased abundance of mRNA encoding one or more LRPIs identified in Table XIV, XVI, XVIII, XX, XXII and XXIV (or any suitable combination) in a test sample relative to a control sample or previously determined reference range indicates the presence of liver response. Any suitable hybridization assay can be used to detect LRPI expression by detecting and/or visualizing mRNA encoding the LRPI (e.g., Northern assays, dot blots, in situ hybridization, etc.).

In another embodiment of the invention, labeled antibodies, derivatives and analogs thereof, which specifically bind to an LRPI can be used for diagnostic purposes, e.g. to detect, diagnose, or monitor liver response. Preferably, liver response is detected in an animal, more preferably in a mammal and most preferably in a human.

Screening Assays

The invention provides methods for identifying active agents (e.g., chemical compounds, proteins or peptides) from a pool of candidate agents that bind to an LRPI or have a stimulatory or inhibitory effect on the expression or activity of an LRPI. The invention also provides methods of identifying active agents that bind to an LRPI-related polypeptide or have an on the expression or activity of an LRPI-related polypeptide. Examples of types of agents include, but are not limited to, nucleic acids (e.g., DNA and RNA), carbohydrates, lipids, proteins, peptides, peptidomimetics, small molecules and other drugs. Agents can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145; U.S. Pat. No.5,738,996; and U.S. Pat. No. 5,807,683, each of which is incorporated herein in its entirety by reference).

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Eng]. 33:2061; and Gallop et al., 1994, J. Med. Chem. 37:1233, each of which is incorporated herein in its entirety by reference.

Libraries of compounds may be presented, e.g., in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310), each of which is incorporated herein in its entirety by reference.

In one embodiment, active agents that interact with (i.e., bind to) an LRPI, an LRPI fragment (e.g. a functionally active fragment), an LRPI-related polypeptide or a fragment of an LRPI-related polypeptide are identified in a cell-based assay system. In accordance with this embodiment, cells expressing an LRPI, a fragment of an LRPI, an LRPI-related polypeptide, or a fragment of an LRPI-related polypeptide are contacted with a candidate agent or a control agent and the ability of the candidate agent to interact with the LRPI is determined. If desired, this assay may be used to screen a plurality (e.g. a library) of candidate agents. The cell, for example, can be of prokaryotic origin (e.g., E. coli) or eukaryotic origin (e.g., yeast or mammalian). Further, the cells can express the LRPI, fragment of the LRPI, LRPI-related polypeptide, or fragment of the LRPI-related polypeptide endogenously or be genetically engineered to express the LRPI, fragment of the LRPI, LRPI-related polypeptide, or fragment of the LRPI-related polypeptide. In some embodiments, the LRPI, fragment of the LRPI, LRPI-related polypeptide, or fragment of the LRPI-related polypeptide, or the test agent is labeled, for example with a radioactive label (such as 32P, 35S or 125I) or a fluorescent label (such as fluorescein isothiocyanate, rhodamine, phycoerythrin, phycocyanin, allophycocyanin, o-phthaldehyde or fluorescamine) to enable detection of an interaction between an LRPI and a candidate agent. The ability of the candidate agent to interact directly or indirectly with an LRPI, a fragment of an LRPI, an LRPI-related polypeptide, or fragment of an LRPI-related polypeptide can be determined by methods known to those of skill in the art. For example, the interaction between a candidate agent and an LRPI, a fragment of an LRPI, an LRPI-related polypeptide, or fragment of an LRPI-related polypeptide can be determined by flow cytometry, a scintillation assay, immunoprecipitation or western blot analysis.

In another embodiment, active agents that interact with (i.e., bind to) an LRPI, an LRPI fragment (e.g., a functionally active fragment) an LRPI-related polypeptide or fragment of an LRPI-related polypeptide are identified in a cell-free assay system. In accordance with this embodiment, a native or recombinant LRPI or fragment thereof, or a native or recombinant LRPI-related polypeptide or fragment thereof is contacted with a candidate agent or a control agent and the ability of the candidate agent to interact with the LRPI or LRPI-related polypeptide is determined. If desired, this assay may be used to screen a plurality (e.g. a library) of candidate agents. Preferably, the LRPI, LRPI fragment, LRPI-related polypeptide or fragment of an LRPI-related polypeptide is first immobilized, by, for example, contacting the LRPI, LRPI fragment, LRPI-related polypeptide or fragment of an LRPI-related polypeptide with an immobilized antibody which specifically recognizes and binds it, or by contacting a purified preparation of the LRPI, LRPI fragment, LRPI-related polypeptide or fragment of an LRPI-related polypeptide with a surface designed to bind proteins. The LRPI, LRPI fragment, LRPI-related polypeptide or fragment of an LRPI-related polypeptide may be partially or completely purified (e.g., partially or completely free of other polypeptides) or part of a cell lysate. Further, the LRPI, LRPI fragment, LRPI-related polypeptide or fragment of an LRPI-related polypeptide may be a fusion protein comprising the LRPI or a biologically active portion thereof, or LRPI-related polypeptide and a domain such as glutathionine-S-transferase. Alternatively, the LRPI, LRPI fragment, LRPI-related polypeptide or fragment of an LRPI-related polypeptide can be biotinylated using techniques well known to those of skill in the art (e.g., biotinylation kit, Pierce Chemicals, Rockford, Ill.). The ability of the candidate agent to interact with an LRPI, LRPI fragment, LRPI-related polypeptide or fragment of an LRPI-related polypeptide can be can be determined by methods known to those of skill in the art.

In another embodiment, a cell-based assay system is used to identify active agents that bind to or modulate the activity of a protein, such as an enzyme, or a biologically active portion thereof, which is responsible for the production or degradation of an LRPI or is responsible for the post-translational modification of an LRPI. In a primary screen, a plurality (e.g., a library) of candidate agents are contacted with cells that naturally or recombinantly express: (i) an LRPI, an isoform of an LRPI, an LRPI homolog an LRPI-related polypeptide, or a biologically active fragment of any of the foregoing; and (ii) a protein that is responsible for processing of the LRPI, LRPI isoform, LRPI homolog, LRPI-related polypeptide or fragment in order to identify agents that modulate the production, degradation, or post-translational modification of the LRPI, LRPI isoform, LRPI homolog, LRPI-related polypeptide or fragment. If desired, agents identified in the primary screen can then be assayed in a secondary screen against cells naturally or recombinantly expressing the specific LRPI of interest. The ability of the candidate agent to modulate the production, degradation or post-translational modification of an LRPI, isoform, homolog or LRPI-related polypeptide can be determined by methods known to those of skill in the art, including without limitation, flow cytometry, a scintillation assay, immunoprecipitation and western blot analysis.

In another embodiment, active agents that competitively interact with (i.e., bind to) an LRPI, LRPI fragment, LRPI-related polypeptide or fragment of an LRPI-related polypeptide are identified in a competitive binding assay. In accordance with this embodiment, cells expressing an LRPI, LRPI fragment, LRPI-related polypeptide or fragment of an LRPI-related polypeptide are contacted with a candidate agent and an agent known to interact with the LRPI, LRPI fragment, LRPI-related polypeptide or fragment of an LRPI-related polypeptide; the ability of the candidate agent to competitively interact with the LRPI, LRPI fragment, LRPI-related polypeptide or fragment of an LRPI-related polypeptide is then determined. Alternatively, active agents that competitively interact with (i.e., bind to) an LRPI, LRPI fragment, LRPI-related polypeptide or fragment of an LRPI-related polypeptide are identified in a cell-free assay system by contacting an LRPI, LRPI fragment, LRPI-related polypeptide or fragment of an LRPI-related polypeptide with a candidate agent and an agent known to interact with the LRPI or LRPI-related polypeptide. As stated above, the ability of the candidate agent to interact with an LRPI, LRPI fragment, LRPI-related polypeptide or fragment of an LRPI-related polypeptide can be determined by methods known to those of skill in the art. These assays, whether cell-based or cell-free, can be used to screen a plurality (e.g., a library) of candidate agents.

In another embodiment, active agents that modulate (i.e., upregulate or downregulate) the expression of an LRPI or an LRPI-related polypeptide are identified by contacting cells (e.g., cells of prokaryotic origin or eukaryotic origin) expressing the LRPI or LRPI-related polypeptide with a candidate agent or a control agent (e.g., phosphate buffered saline (PBS)) and determining the expression of the LRPI, LRPI-related polypeptide, mRNA encoding the LRPI, or mRNA encoding the LRPI-related polypeptide. The level of expression of a selected LRPI, LRPI-related polypeptide, mRNA encoding the LRPI, or mRNA encoding the LRPI-related polypeptide in the presence of the candidate agent is compared to the level of expression of the LRPI, LRPI-related polypeptide, mRNA encoding the LRPI, or mRNA encoding the LRPI-related polypeptide in the absence of the candidate agent (e.g., in the presence of a control agent). The candidate agent can then be identified as a modulator of the expression of the LRPI or LRPI-related polypeptide based on this comparison. For example, when expression of the LRPI or mRNA is significantly greater in the presence of the candidate agent than in its absence, the candidate agent is identified as a stimulator of expression of the LRPI or mRNA. Alternatively, when expression of the LRPI or mRNA is significantly less in the presence of the candidate agent than in its absence, the candidate agent is identified as an inhibitor of the expression of the LRPI or mRNA. The level of expression of an LRPI or the mRNA that encodes it can be determined by methods known to those of skill in the art based on the present description. For example, mRNA expression can be assessed by Northern blot analysis or RT-PCR, and protein levels can be assessed by western blot analysis.

In another embodiment, active agents that modulate the activity of an LRPI or an LRPI-related polypeptide are identified by contacting a preparation containing the LRPI or LRPI-related polypeptide, or cells (e.g., prokaryotic or eukaryotic cells) expressing the LRPI or LRPI-related polypeptide with a candidate agent and determining the ability of the candidate agent to modulate the activity of the LRPI or LRPI-related polypeptide. The activity of an LRPI or an LRPI-related polypeptide can be assessed by detecting a downstream effector of the LRPI or LRPI-related polypeptide (e.g., intracellular Ca²⁺, diacylglycerol, IP3, etc.), detecting catalytic or enzymatic activity of the target on a suitable substrate, detecting the induction of a reporter gene (e.g., a regulatory element that is responsive to an LRPI or an LRPI-related polypeptide and is operably linked to a nucleic acid encoding a detectable marker, e.g., luciferase), or detecting a cellular response, for example, cellular differentiation, or cell proliferation, as the case may be. Based on the present description, techniques known to those of skill in the art can be used for measuring these activities (see, e.g., U.S. Pat. No. 5,401,639, which is incorporated herein by reference in its entirety). The candidate agent can then be identified as a modulator of the activity of an LRPI or LRPI-related polypeptide by comparing the effects of the candidate agent to a control agent. Suitable controls include phosphate buffered saline (PBS) and normal saline (NS).

In another embodiment, active agents that modulate the expression, activity or both the expression and activity of an LRPI or LRPI-related polypeptide are identified in an animal model. Examples of suitable animals include, but are not limited to, mice, rats, rabbits, pigs, monkeys and guinea pigs. Preferably, the animal used represents a model of liver response. For instance, animals may be treated with various drugs to induce liver response as described in the Examples herein. In accordance with this embodiment, the candidate agent or a control agent is administered in any suitable way or manner (e.g., orally, rectally or parenterally such as intraperitoneally or intravenously) to a suitable animal and the effect on the expression, activity or both expression and activity of the LRPI or LRPI-related polypeptide is determined. Changes in the expression of an LRPI or LRPI-related polypeptide can be assessed by any suitable method, such as the methods described above based on the present description.

In yet another embodiment, an LRPI or LRPI-related polypeptide is used as a “bait protein” in a two-hybrid assay or three hybrid assay to identify other proteins that bind to or interact with an LRPI or LRPI-related polypeptide (see, e.g., U.S. Pat. No. 5,283,317; Zervos et al. (1993) Cell 72:223-232; Madura et al. (1993) J. Biol. Chem. 268:12046-12054; Bartel et al. (1993) BioTechniques 14:920-924; Twabuchi et al. (1993) Oncogene 8:1693-1696; and PCT Publication No. WO 94/10300). As those skilled in the art will appreciate, such binding proteins are also likely to be involved in the propagation of signals by the LRPIs of the inventions as, for example, upstream or downstream elements of a signaling pathway involving the LRPIs of the invention.

As those skilled in the art will appreciate, Table XXVII enumerates scientific publications describing suitable assays for detecting or quantifying enzymatic or binding activity of an LRPI, an LRPI analog, an LRPI-related polypeptide, or a fragment of any of the foregoing. Each such reference is hereby incorporated in its entirety. In a preferred embodiment, an assay referenced in Table XXVI is used in the screens and assays described herein, for example, to screen for or to identify an agent that modulates the activity of (or that modulates both the expression and activity of) an LRPI, LRPI analog, or LRPI-related polypeptide, or fragment of any of the foregoing.

TABLE XXVII
Detection and/or quantification assays for LRPI binding activity

| LRF# | LRPI# | Description | Scientific References For Assays |
|---|---|---|---|
| LRF-164, LRF- | LRPI-164.1, | COMPLEMENT | “Time-resolved immunofluorometric |
| 165, LRF-166, | LRPI-165.1, | C3 PRECURSOR | assay of complement C3: application |
| LRF-217, LRF- | LRPI-166.1, | [CONTAINS: | to cerebrospinal fluid.” Clin Chem |
| 221, LRF-222, | LRPI-217.3, | C3A | 1993 Feb 39:2 309-12 |
| LRF-266, LRF- | LRPI-221.1, | ANAPHYLATOXIN] | “A fluorimetric assay for native C3. |
| 363, LRF-364, | LRPI-222.1, | | The hemolytically active form of the |
| | LRPI-266.1, | | third component of human |
| | LRPI-363.1, | | complement.” |
| | LRPI-364.1 | | J Immunol Methods Aug 24, 1987 |
| | | | 41825.2506944444 |
| LRF-167, LRF- | LRPI-167.1, | COMPLEMENT | “Development of a rapid kinetic assay |
| 224, LRF-296, | LRPI-224.1, | C4 PRECURSOR | for the function of the classical |
| LRF-223 | LRPI-296.1, | [CONTAINS: | pathway of the complement system |
| | LRPI-223.1 | C4A | and for C2 and C4.” |
| | | ANAPHYLATOXIN] | J Clin Lab Immunol 1986 Dec 21:4 |
| | | | 201-7 |
| LRF-21, LRF- | LRPI-21.1, LRPI- | GLYCERALDEHYDE | “Alternation of glyceraldehyde-3- |
| 120 | 120.1 | 3- | phosphate dehydrogenase activity and |
| | | PHOSPHATE | messenger mRNA content by |
| | | DEHYDROGENASE, | androgen in human prostate |
| | | LIVER (EC | carcinoma cells”, (1995), Cancer Res. |
| | | 1.2.1.12) | 55(19, pp4234-6 |
| | | | “Increased membrane activity of |
| | | | glyceraledhyde 3-phosphate |
| | | | dehydrogenase in erythrocytes of |
| | | | patients with homozygous sickle cell |
| | | | anaemia”, (1992), Clin Chim Acta |
| | | | 209(3), pp189-95 |
| | | | “Heterogeneity of glyceraldehyde-3- |
| | | | phosphate dehydrogenase from human |
| | | | brain” (1988), 954(3), pp309-24 |
| LRF-177 | LRPI-177.1 | PLASMA | “Assay of human transthyretin-bound |
| | | RETINOL- | holo-retinol-binding protein with |
| | | BINDING | reversed-phase high-performance |
| | | PROTEIN | liquid chromatography.” J Chromatogr |
| | | PRECURSOR | Jul 5, 1991 567:2 369-80 |
| | | (PRBP) (RBP) | “Liquid-chromatographic assay for |
| | | | free and transthyretin-bound retinol- |
| | | | binding protein in serum from normal |
| | | | humans.” Clin Chem 1989 Apr 35:4 |
| | | | 582-6 |
| LRF-236, LRF- | LRPI-236.1, | TRANSTHYRETIN | “Rational design of potent human |
| 373 | LRPI-373.1 | PRECURSOR | transthyretin amyloid disease |
| | | (PREALBUMIN) | inhibitors.” Nat Struct Biol. 2000 |
| | | (TBPA) (TTR) | Apr;7(4):312-21. |
| | | (ATTR) | “Structure-Based Design of N-Phenyl |
| | | | Phenoxazine Transthyretin Amyloid |
| | | | Fibril Inhibitors” J. Am. Chem. Soc. |
| | | | 122, 2178-2192, 2000 |

This invention further provides agents, including novel agents, identified by the above-described screening assays and uses thereof for treatments as described herein.

Therapeutic Uses Of LRPIs

The invention provides for treatment or prevention of various diseases and disorders by administration of an active agent. Such agents include but are not limited to: LRPIs, LRPI analogs, LRPI-related polypeptides and derivatives (including fragments) thereof; antibodies to the foregoing; nucleic acids encoding LRPIs, LRPI analogs, LRPI-related polypeptides and fragments thereof; antisense nucleic acids to a gene encoding an LRPI or LRPI-related polypeptide; and modulators (e.g., agonists and antagonists) of a gene encoding an LRPI or LRPI-related polypeptide. An important feature of the present invention is the identification of genes encoding LRPIs involved in liver response. Liver response can be treated (e.g. to ameliorate symptoms or to retard onset or progression) by administration of an active agent that promotes function or expression of one or more LRPIs that are decreased in the blood or liver of liver response subjects with liver response, or by administration of an active agent that reduces function or expression of one or more LRPIs that are increased in the blood or liver of subjects with liver response. In one embodiment of the invention, liver response is treated or prevented by administration of a compound that agonises (stimulates) the level(s) and/or function(s) of one or more LRPIs which are decreased in the blood or liver of subjects having liver response as compared with blood or liver of subjects free from liver response. In another embodiment of the invention, liver response is treated or prevented by administration of a compound that antagonizes (inhibits) the level(s) and/or function(s) of one or more LRPIs which are elevated in the blood or liver of subjects having liver response as compared with blood or liver of subjects free from liver response.

In a specific embodiment, LRPI expression is inhibited by use of LRPI antisense nucleic acids. The present invention provides the therapeutic or prophylactic use of nucleic acids comprising at least six nucleotides that are antisense to a gene or cDNA encoding an LRPI or a portion thereof. As used herein, an LRPI “antisense” nucleic acid refers to a nucleic acid capable of hybridizing by virtue of some sequence complementarity to a portion of an RNA (preferably mRNA) encoding an LRPI.

In a further embodiment, one or more antibodies each specifically binding to an LRPI are administered alone or in combination with one or more additional active agents or treatments. In one embodiment the active agent is administered alone, in a further embodiment, one or more active agents which modulate an LRPI are administered in combination with one or more additional active agents or treatments. In a further embodiment, nucleic acids comprising a sequence encoding an LRPI, an LRPI fragment, LRPI-related polypeptide or fragment of an LRPI-related polypeptide, are administered to promote LRPI function by way of gene therapy. Gene therapy refers to administration to a subject of an expressed or expressible nucleic acid. In this embodiment, the nucleic acid produces its encoded polypeptide that mediates a therapeutic effect by promoting LRPI function.

Any of the methods for gene therapy available in the art can be used according to the present invention. In another embodiment, symptoms of liver response may be ameliorated by decreasing the level of an LRPI or LRPI activity by using gene sequences encoding the LRPI in conjunction with well-known gene “knock-out,” ribozyme or triple helix methods to decrease gene expression of an LRPI. In this approach ribozyme or triple helix molecules are used to modulate the activity, expression or synthesis of the gene encoding the LRPI, and thus to ameliorate the symptoms of liver response.

Preferably, a biological product such as an antibody is allogeneic to the subject to which it is administered. In a preferred embodiment, a human LRPI or a human LRPI-related polypeptide, a nucleotide sequence encoding a human LRPI or a human LRPI-related polypeptide, or an antibody to a human LRPI or a human LRPI-related polypeptide, is administered to a human subject for treatment (e.g. to ameliorate symptoms or to retard onset or progression).

Therapeutic and Prophylactic Compositions and Their Use

The invention provides methods of treatment (and prophylaxis) comprising administering to a subject an effective amount of an active agent of the invention. In a preferred aspect, the agent is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human. In a specific embodiment, a non-human mammal is the subject.

Formulations and methods of administration that can be employed are described below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In another embodiment, the compound can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp.353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). Other controlled release systems are discussed in the review by Langer (1990, Science 249:1527-1533).

In a specific embodiment where the compound of the invention is a nucleic acid encoding a protein, the nucleic acid can be administered ini vivo to promote expression of its encoded protein, by constructing it as part of an appropriate nucleic acid expression vector and administering it so that it becomes intracellular, e.g., by use of a retroviral vector (see U.S. Pat. No.4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier, vehicle or diluent. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment of liver response can be determined by standard clinical techniques. In addition, in vitro assays may optionally be employed to help identify optimal dosage ranges. The precise dose to be employed in the formulation will also depend on the route of administration, and the severity of the liver response, and should be decided according to the judgment of the practitioner and each subject's circumstances.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### EXAMPLE

Identification of Proteins Differentially Expressed in the Blood or Liver in Liver Response

Samples from animals treated with various drugs at various timepoints and various dose levels can be collected, prepared, and analysed as described herein. In the studies provided herein, animals were treated with acetaminophen, alpha-naphthylisothiocyanate (ANIT), phenobarbital, Wy-14,643, and valproic acid. Using the following procedure, proteins in liver tissue and serum/plasma from 5 control animals and 5 animals having liver response induced through drug treatment were separated by isoelectric focusing followed by SDS-PAGE and analyzed. Samples were taken and analyzed at three different time points designated “early”, “full manifestation” and “late”. Parts 6.1.1 to 6.1.9 (inclusive) of the procedure set forth below are hereby designated as the “Reference Protocol”.

Materials and Methods

Blood Sample Preparation

Approximately 200 μl venous blood was collected at the time of necropsy in serum seperation tubes and serum obtained by centrifugation for 3 minutes at 11,000 rpm using a Biofuge A centrifuge. 100 μl of the resulting serum was added to 15 μl protease inhibitor, vortexed and frozen at −80° C.

A protein assay (Pierce (Rockford, Ill.) BCA Cat #23225) was performed on each serum/plasma sample as received. Prior to protein separation, each sample was processed for selective depletion of certain proteins, in order to enhance and simplify protein separation and facilitate analysis by removing proteins that may interfere with or limit analysis of proteins of interest. See International Patent Application No. PCT/GB99/01742, filed Jun. 1, 1999, which is incorporated by reference in its entirety, with particular reference to pages 3 and 6.

Removal of albumin, haptoglobin, transferrin and immunoglobin G (IgG) from serum/plasma (“serum/plasma depletion”) was achieved by an affinity chromatography purification step in which the sample was passed through a series of ‘Hi-Trap’ columns containing immobilized antibodies for selective removal of albumin, haptoglobin and transferrin, and protein G for selective removal of immunoglobin G. Two affinity columns in a tandem assembly were prepared by coupling antibodies to protein G-sepharose contained in Hi-Trap columns (Protein G-Sepharose Hi-Trap columns (1 ml) Pharmacia Cat. No. 17-0404-01). This was done by circulating the following solutions sequentially through the columns: (1) Dulbecco's Phosphate Buffered Saline (Gibco BRL Cat. No. 14190-094); (2) concentrated antibody solution; (3) 200 mM sodium carbonate buffer, pH 8.35; (4) cross-linking solution (200 mM sodium carbonate buffer, pH 8.35, 20 mM dimethylpimelimidate); and (5) 500 mM ethanolamine, 500 mM NaCl. A third (un-derivatised) protein G Hi-Trap column was then attached to the lower end of the tandem column assembly.

The chromatographic procedure was automated using an Akta Fast Protein Liquid Chromatography (FPLC) System such that a series of up to seven runs could be performed sequentially. The samples were passed through the series of 3 Hi-Trap columns in which the affinity chromatography media selectively bind the above proteins thereby removing them from the sample. Fractions (typically 3 ml per tube) were collected of unbound material (“Flowthrough fractions”) that eluted through the column during column loading and washing stages and of bound proteins (“Bound/Eluted fractions”) that were eluted by step elution with Immunopure Gentle Ag/Ab Elution Buffer (Pierce Cat. No. 21013). The eluate containing unbound material was collected in fractions which were pooled, desalted/concentrated by centrifugal ultrafiltration and stored to await further analysis by 2D PAGE.

A volume of depleted serum/plasma containing approximately 300 μg of total protein was aliquoted and an equal volume of 10% (w/v) SDS (Fluka 71729), 2.3% (w/v) dithiothreitol (BDH 443852A) was added. The sample was heated at 95° C. for 5 mins, and then allowed to cool to 20° C . 125 μl of the following buffer was then added to the sample:

8M urea (BDH 452043w)

4% CHAPS (Sigma C3023)

65 mM dithiotheitol (DTT)

2% (v/v) Resolytes 3.5-10 (BDH 44338 2×)

This mixture was vortexed, and centrifuged at 13000 rpm for 5 mins at 15° C., and the supernatant was analyzed by isoelectric focusing.

Liver Sample Preparation

At the time of necropsy, approximately 2 g section of the liver's median lobe was removed to a conical tube and quick-frozen in liquid nitrogen. The frozen liver section was then pulverized for 1 minute using a freezer mill as follows:

Freezer Mill Specifications:

SPEX CertiPrep 203 Norcross Ave., Metuchen, NJ 08840
1-800-LAB-SPEX (1-800-522-7739)

| | |
|---|---|
| 6750 Freezer Mill | Cat. #: 6750 |
| Grinding Vial Set (Tubes) | Cat. #: 6701 |
| Extractor and Vial Opener Set | Cat. #: 6704 |

Freezer Mill Operating Procedures:

1. Fill the chamber with liquid nitrogen up to the indicated line approximately 1 hour before use to allow the chamber to cool. Refill the chamber as necessary.

2. Chill the grinding vial in liquid nitrogen.

3. Add approximately 2 g frozen liver sample to the grinding vial. The frozen liver pieces must be small enough to fit into the grinding vial and still allow the stainless steel impactor to move freely.

4. Cap the grinding vial and place into the chilled chamber containing liquid nitrogen. Close the chamber.

5. Ensure the chamber is filled with liquid nitrogen when running a sample. Do not run dry.

6. Set the timer to I minute and press start. The mill will begin pulverizing the sample.

7. When cycle is complete, open the chamber and remove the grinding vial with the extractor handle. Twist the handle to open the vial. Work quickly to keep the sample from thawing.

8. Pour the resulting powder into a labeled tube. An ice-cold spatula may be used to aid sample recovery.

9. Immediately place the labeled tube in liquid nitrogen and store at −80 ° C.

10. When all samples have been pulverized, clean the grinding vials and dry completely:

11. use warm soap and water to clean vials. Do not use organic solvents to clean the vials.

12. Leave the chamber open to allow the liquid nitrogen to evaporate.

13. Unplug the freezer mill when not in use.

Approximately 10-20 mg pulverized liver was solubilized with 10 ul protease inhibitor and 1.5 ml lysis solution and vortexed well. The sample was allowed to remain at room temperature for 10 minutes, was vortexed again, and then pelleted using an Eppendorf 5417R Centrifuge for 10 minutes at 13000g at 15 EC. The resulting supernatant was frozen at −80° C.

The sample was heated at 95° C. for 5 mins, and then allowed to cool to 20° C. 125 μl of the following buffer was then added to the sample:

8M urea (BDH 452043w)

4% CHAPS (Sigma C3023)

65 mM dithiotheitol (DTT)

2% (v/v) Resolytes 3.5-10 (BDH 44338 2×)

This mixture was vortexed, and centrifuged at 13000 rpm for 5 mins at 15° C., and the supernatant was analysed by isoelectric focusing. An assay for protein content was carried out on the final sample (Pierce BCA Cat #23225).

Isoelectric Focusing

Isoelectric focusing (IEF), was performed using the Immobiline® DryStrip Kit (Pharmacia BioTech), following the procedure described in the manufacturer's instructions, see Instructions for Immobilineg DryStrip Kit, Pharmacia, #18-1038-63, Edition AB (incorporated herein by reference in its entirety). Immobilized pH Gradient (IPG) strips (18 cm, pH 3-10 non-linear strips; Pharmacia Cat. #17-1235-01) were rehydrated overnight at 20° C. in a solution of 8M urea, 2% (w/v) CHAPS, 10 mM DTT, 2% (v/v) Resolytes 3.5-10, as described in the Immobiline DryStrip Users Manual. For IEF, 50 μl of supernatant (prepared as above) was loaded onto a strip, with the cup-loading units being placed at the basic end of the strip. The loaded gels were then covered with mineral oil (Pharmacia 17-3335-01) and a voltage was immediately applied to the strips according to the following profile, using a Pharmacia EPS3500XL power supply (Cat 19-3500-01):

Initial voltage=300V for 2 hrs

Linear Ramp from 300V to 3500V over 3 hrs

Hold at 3500V for 19 hrs

For all stages of the process, the current limit was set to 10 mA for 12 gels, and the wattage limit to 5W. The temperature was held at 20° C. throughout the run.

Gel Equilibration and SDS-PAGE

After the final 19 hr step, the strips were immediately removed and immersed for 10 mins at 20° C. in a first solution of the following composition: 6M urea; 2% (w/v) DTT; 2% (w/v) SDS; 30% (v/v) glycerol (Fluka 49767); 0.05M Tris/HCl, pH 6.8 (Sigma Cat T-1503). The strips were removed from the first solution and immersed for 10 mins at 20° C. in a second solution of the following composition: 6M urea; 2% (w/v) iodoacetamide (Sigma 1-6125); 2% (w/v) SDS; 30% (v/v) glycerol; 0.05M Tris/HCl, pH 6.8. After removal from the second solution, the strips were loaded onto supported gels for SDS-PAGE according to Hochstrasser et al., 1988, Analytical Biochemistry 173: 412-423 (incorporated herein by reference in its entirety), with modifications as specified below.

Preparation of Supported Gels

The gels were cast between two glass plates of the following dimensions: 23 cm wide×24 cm long (back plate); 23 cm wide×24 cm long with a 2 cm deep notch in the central 19 cm (front plate). To promote covalent attachment of SDS-PAGE gels, the back plate was treated with a 0.4% solution of γ-methacryl-oxypropyltrimethoxysilane in ethanol (BindSilane™; Pharmacia Cat. #17-1330-01). The front plate was treated with a 2% solution of dimethyldichlorosilane dissolved in octamethyl cyclo-octasilane (RepelSilane™; Pharmacia Cat. #17-1332-01) to reduce adhesion of the gel. Excess reagent was removed by washing with water, and the plates were allowed to dry. At this stage, both as identification for the gel, and as a marker to identify the coated face of the plate, an adhesive bar-code was attached to the back plate in a position such that it would not come into contact with the gel matrix.

The dried plates were assembled into a casting box with a capacity of 13 gel sandwiches. The top and bottom plates of each sandwich were spaced by means of 1 mm thick spacers, 2.5 cm wide. The sandwiches were interleaved with acetate sheets to facilitate separation of the sandwiches After gel polymerization Casting was then carried out according to Hochstrasser et al., op. cit.

A 9-16% linear polyacrylamide gradient was cast, extending up to a point 2 cm below the level of the notch in the front plate, using the Angelique gradient casting system (Large Scale Biology). Stock solutions were as follows. Acrylamide (40% in water) was from Serva (Cat. #10677). The cross-linking agent was PDA (BioRad 161-0202), at a concentration of 2.6% (w/w) of the total starting monomer content. The gel buffer was 0.375M Tris/HCl, pH 8.8. The polymerization catalyst was 0.05% (v/v) TEMED (BioRad 161-0801), and the initiator was 0.1% (w/v) APS (BioRad 161-0700). No SDS was included in the gel and no stacking gel was used. The cast gels were allowed to polymerize at 20° C. overnight, and then stored at 4° C. in sealed polyethylene bags with 6 ml of gel buffer, and were used within 4 weeks.

SDS-PAGE

A solution of 0.5% (w/v) agarose (Fluka Cat 05075) was prepared in running buffer (0.025 M Tris, 0.198 M glycine (Fluka 50050), 1% (w/v) SDS, supplemented by a trace of bromophenol blue). The agarose suspension was heated to 70° C. with stirring, until the agarose had dissolved. The top of the supported 2nd D gel was filled with the agarose solution, and the equilibrated strip was placed into the agarose, and tapped gently with a palette knife until the gel was intimately in contact with the 2nd D gel. The gels were placed in the 2nd D running tank, as described by Amess et al., 1995, Electrophoresis 16:1255-1267 (incorporated herein by reference in its entirety). The tank was filled with running buffer (as above) until the level of the buffer was just higher than the top of the region of the 2nd D gels which contained polyacrylamide, so as to achieve efficient cooling of the active gel area. Running buffer was added to the top buffer compartments formed by the gels, and then voltage was applied immediately to the gels using a Consort E-833 power supply. For 1 hour, the gels were run at 20 mA/gel. The wattage limit was set to 150W for a tank containing 6 gels, and the voltage limit was set to 600V. After 1 hour, the gels were then run at 40 mA/gel, with the same voltage and wattage limits as before, until the bromophenol blue line was 0.5 cm from the bottom of the gel. The temperature of the buffer was held at 16° C. throughout the run. Gels were not run in duplicate.

Staining

Upon completion of the electrophoresis run, the gels were immediately removed from the tank for fixation. The top plate of the gel cassette was carefully removed, leaving the gel bonded to the bottom plate. The bottom plate with its attached gel was then placed into a staining apparatus, which can accommodate 12 gels. The gels were completely immersed in fixative solution of 40% (v/v) ethanol (BDH 28719), 10% (v/v) acetic acid (BDH 100016X), 50% (v/v) water (MilliQ-Millipore), which was continuously circulated over the gels. After an overnight incubation, the fixative was drained from the tank, and the gels were primed by immersion in 7.5% (v/v) acetic acid, 0.05% (w/v) SDS, 92.5% (v/v) water for 30 mins. The priming solution was then drained, and the gels were stained by complete immersion for 4 hours in a staining solution of Pyridinium, 4-[2-[4-(dipentylamino)-2-trifluoromethylphenyl]ethenyl]-1-(sulfobutyl)-, inner salt, prepared by diluting a stock solution of this dye (2mg/ml in DMSO) in 7.5% (v/v) aqueous acetic acid to give a final concentration of 1.2 mg/l; the staining solution was vacuum filtered through a 0.4 μm filter (Duropore) before use.

Imaging of the Gel

A computer-readable output was produced by imaging the fluorescently stained gels with the preferred scanner described in section 5.2, supra. This scanner has a gel carrier with four integral fluorescent markers (Designated M1, M2, M3, M4) that are used to correct the image geometry and are a quality control feature to confirm that the scanning has been performed correctly.

For scanning, the gels were removed from the stain, rinsed with water and allowed to air dry briefly, and imaged on the scanner. After imaging, the gels were sealed in polyethylene bags containing a small volume of staining solution, and then stored at 4° C.

Digital Analysis of the Data

The data were processed as described in U.S. Pat. No. 6,278,794 at Sections 5.4 and 5.5 (incorporated herein by reference), as set forth more particularly below.

The output from the scanner was first processed using the MELANIE® II 2D PAGE analysis program (Release 2.2, 1997, BioRad Laboratories, Hercules, California, Cat. #170-7566) to autodetect the registration points, M1, M2, M3 and M4; to autocrop the images (i.e., to eliminate signals originating from areas of the scanned image lying outside the boundaries of the gel, e.g. the reference frame); to filter out artifacts due to dust; to detect and quantify features; and to create image files in GIF format. Features were detected using the following parameters:

For depleted serum sample analysis:

Smooths=1

Laplacian threshold=10

Partials threshold=10

Saturation=100

Peakedness increase=0

Minimum Perimeter=10

For depleted liver lysate sample analysis:

Smooths=1

Laplacian threshold=100

Partials threshold=50

Saturation=100

Peakedness increase=0

Minimum Perimeter=10

Assignment of pI and MW Values

Landmark identification was used to determine the pI and MW of features detected in the images.

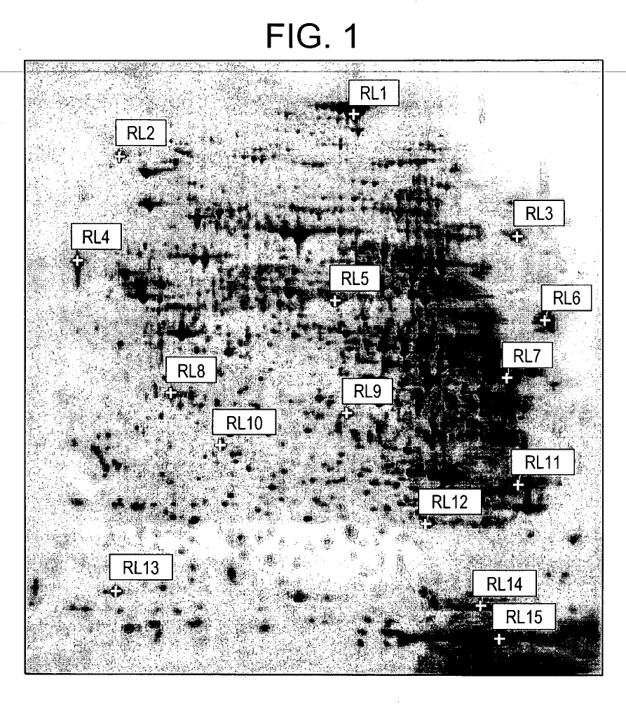

Fifteen landmark features, designated RL1 to RL15, were identified in a standard liver tissue lysate image. These landmark features are identified in FIG. 1 and were assigned the pI and/or MW values identified in Table VII.

Table XXVIII. Landmark Features Used in this Study for Liver Tissue Lysate

| Landmark | | |
|---|---|---|
| Name | pI | Mw (Da) |
| RL1 | 6.42 | 160697 |
| RL2 | 4.75 | 110676 |
| RL3 | — | 64312 |
| RL4 | 4.50 | 57645 |
| RL5 | 6.24 | — |
| RL6 | 10.17 | 47685 |
| RL7 | 9.16 | 37544 |
| RL8 | 5.11 | — |
| RL9 | — | 32515 |
| RL10 | 5.51 | 28518 |
| RL11 | — | 25116 |
| RL12 | 7.40 | 19903 |
| RL13 | — | 13746 |
| RL14 | 8.68 | — |
| RL15 | — | 11379 |

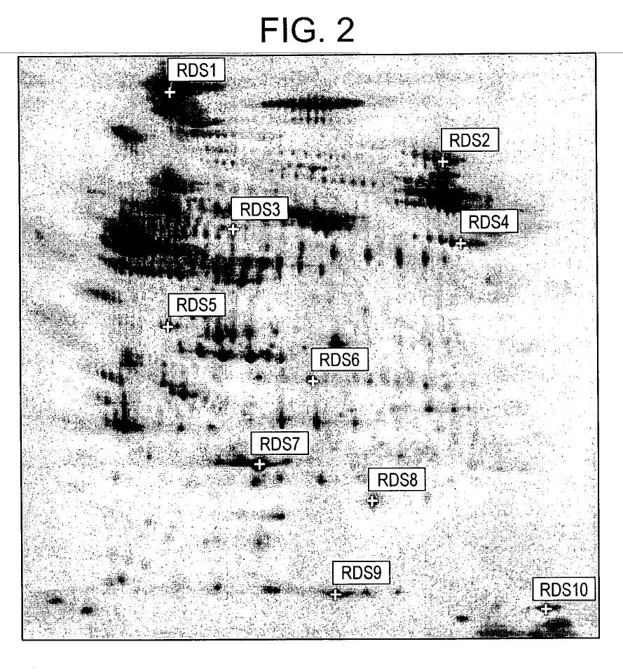

Ten landmark features, designated RDS1 to RDS10, were identified in a standard serum image. These landmark features are identified in FIG. 2 and were assigned the pI and/or MW values identified in Table VIII.

Table XXIX. Landmark Features Used in this Study for Serum

| Landmark | | |
|---|---|---|
| Name | pI | Mw (Da) |
| RDS1 | — | 150000 |
| RDS2 | — | 104343 |
| RDS3 | 5.39 | 68471 |
| RDS4 | 7.54 | — |
| RDS5 | 4.97 | 41493 |
| RDS6 | 5.92 | 32948 |
| RDS7 | 5.55 | 22781 |
| RDS8 | 6.44 | 18874 |
| RDS9 | 6.12 | 11903 |
| RDS10 | 9.10 | — |

As many of these landmarks as possible were identified in each gel image of the dataset. Each feature in the study gels was then assigned a pI value by linear interpolation or extrapolation (using the MELANIE®-II software) to the two nearest landmarks, and was assigned a MW value by linear interpolation or extrapolation (using the MELANIE®-II software) to the two nearest landmarks.

Matching With Primary Master Image

Images were edited to remove gross artifacts such as dust, to reject images which had gross abnormalities such as smearing of protein features, or were of too low a loading or overall image intensity to allow identification of more than the most intense features, or were of too poor a resolution to allow accurate detection of features. Images were then compared by pairing with one common image from the whole sample set. This common image, the “primary master image”, was selected on the basis of protein load (maximum load consistent with maximum feature detection), a well resolved myoglobin region, (myoglobin was used as an internal standard), and general image quality. Additionally, the primary master image was chosen to be an image which appeared to be generally representative of all those to be included in the analysis. (This process by which a primary master gel was judged to be representative of the study gels was rechecked by the method described below and in the event that the primary master gel was seen to be unrepresentative, it was rejected and the process repeated until a representative primary master gel was found.)

Each of the remaining study gel images was individually matched to the primary master image such that common protein features were paired between the primary master image and each individual study gel image as described below.

Cross-matching Between Samples

To facilitate statistical analysis of large numbers of samples for purposes of identifying features that are differentially expressed, the geometry of each study gel was adjusted for maximum alignment between its pattern of protein features, and that of the primary master, as follows. Each of the study gel images was individually transformed into the geometry of the primary master image using a multi-resolution warping procedure. This procedure corrects the image geometry for the distortions brought about by small changes in the physical parameters of the electrophoresis separation process from one sample to another. The observed changes are such that the distortions found are not simple geometric distortions, but rather a smooth flow, with variations at both local and global scale.

The fundamental principle in multi-resolution modeling is that smooth signals may be modeled as an evolution through ‘scale space’, in which details at successively finer scales are added to a low resolution approximation to obtain the high resolution signal. This type of model is applied to the flow field of vectors (defined at each pixel position on the reference image) and allows flows of arbitrary smoothness to be modeled with relatively few degrees of freedom. Each image is first reduced to a stack, or pyramid, of images derived from the initial image, but smoothed and reduced in resolution by a factor of 2 in each direction at every level (Gaussian pyramid) and a corresponding difference image is also computed at each level, representing the difference between the smoothed image and its progenitor (Laplacian pyramid). Thus the Laplacian images represent the details in the image at different scales.

To estimate the distortion between any 2 given images, a calculation was performed at level 7 in the pyramid (i.e., after 7 successive reductions in resolution). The Laplacian images were segmented into a grid of 16×16 pixels, with 50% overlap between adjacent grid positions in both directions, and the cross correlation between corresponding grid squares on the reference and the test images was computed. The distortion displacement was then given by the location of the maximum in the correlation matrix. After all displacements had been calculated at a particular level, they were interpolated to the next level in the pyramid, applied to the test image, and then further corrections to the displacements were calculated at the next scale.

The warping process brought about good alignment between the common features in the primary master image, and the images for the other samples. The MELANIE®II 2D PAGE analysis program was used to calculate and record approximately 500-700 matched feature pairs between the primary master and each of the other images. The accuracy of this program was significantly enhanced by the alignment of the images in the manner described above. To improve accuracy still further, all pairings were finally examined by eye in the MelView interactive editing program and residual recognizably incorrect pairings were removed. Where the number of such recognizably incorrect pairings exceeded the overall reproducibility of the Preferred Technology (as measured by repeat analysis of the same biological sample) the gel selected to be the primary master gel was judged to be insufficiently representative of the study gels to serve as a primary master gel. In that case, the gel chosen as the primary master gel was rejected, and different gel was selected as the primary master gel, and the process was repeated.

All the images were then added together to create a composite master image, and the positions and shapes of all the gel features of all the component images were super-imposed onto this composite master as described below.

Once all the initial pairs had been computed, corrected and saved, a second pass was performed whereby the original (unwarped) images were transformed a second time to the geometry of the primary master, this time using a flow field computed by smooth interpolation of the multiple tie-points defined by the centroids of the paired gel features. A composite master image was thus generated by initializing the primary master image with its feature descriptors. As each image was transformed into the primary master geometry, it was digitally summed pixel by pixel into the composite master image, and the features that had not been paired by the procedure outlined above were likewise added to the composite master image description, with their centroids adjusted to the master geometry using the flow field correction.

The final stage of processing was applied to the composite master image and its feature descriptors, which now represent all the features from all the images in the study transformed to a common geometry. The features were grouped together into linked sets or “clusters”, according to the degree of overlap between them. Each cluster was then given a unique identifying index, the molecular cluster index (MCI).

An MCI identifies a set of matched features on different images. Thus an MCI represents a protein or proteins eluting at equivalent positions in the 2D separation in different samples.

Construction of Profiles

After matching all component gels in the study to the final composite master image, the intensity of each feature was measured and stored. The end result of this analysis was the generation of a digital profile which contained, for each identified feature: 1) a unique identification code relative to corresponding feature within the composite master image (MCI), 2) the x, y coordinates of the features within the gel, 3) the isoelectric point (pI) of the LRFs, 4) the apparent molecular weight (MW) of the LRFs, 5) the signal value, 6) the standard deviation for each of the preceding measurements, and 7) a method of linking the MCI of each feature to the master gel to which this feature was matched. By virtue of a Laboratory Information Management System (LIMS), this MCI profile was traceable to the actual stored gel from which it was generated, so that proteins identified by computer analysis of gel profile databases could be retrieved. The LIMS also permitted the profile to be traced back to an original sample or patient.

Statistical Analysis of the Profiles

The Wilcoxon Rank Sum Test was used to identify LRFs from the MCIs within the mastergroup. This test was performed between the control and the liver response samples for each MCI basis at three time points designated “early”, “full manifestation”, and “late”. The MCIs which recorded a p-value less than or equal to 0.05 were selected as statistically significant LDFs with 95% selectivity.

Using the Wilcoxon rank sum test, MCIs showing significant differences between the control samples and the liver response samples, with a recorded p-value less than or equal to 0.05 (95% confidence) were classified according to the following sets as shown in the Venn diagram illustration:

| | Time points at which MCIs within the set show a |
|---|---|
| Set Number | statistically significant difference* |
| 1 | EARLY |
| 2 | EARLY & FULL MANIFESTATION |
| 3 | EARLY, FULL MANIFESTATION & LATE |
| 4 | LATE |
| 5 | FULL MANIFESTATION |
| 6 | FULL MANIFESTATION & LATE |
| 7 | EARLY & LATE |

*MCIs are identified as having statistically significant differences between control samples and liver response samples where the MCIs have a p value of less than or equal to 0.05 (95% confidence).

Recovery and Analysis of Selected Proteins

Proteins in LRFs were robotically excised and processed to generate tryptic digest peptides. Tryptic peptides were analyzed by mass spectrometry using a PerSeptive Biosystems Voyager-DETM STR Matrix-Assisted Laser Desorption Ionization Time-of-Flight (MALDI-TOF) mass spectrometer, and selected tryptic peptides were analyzed by tandem mass spectrometry (MS/MS) using a Micromass Quadrupole Time-of-Flight (Q-TOF) mass spectrometer (Micromass, Altrincham, U.K.) equipped with a nanoflow™ electrospray Z-spray source. For partial amino acid sequencing and identification of APIs uninterpreted tandem mass spectra of tryptic peptides were searched using the SEQUEST search program (Eng et al., 1994, J. Am. Soc. Mass Spectrom. 5:976-989), version v.C.1. Criteria for database identification included: the cleavage specificity of trypsin; the detection of a suite of a, b and y ions in peptides returned from the database, and a mass increment for all Cys residues to account for carbamidomethylation. The database searched was database constructed of protein entries in the non-redundant database held by the National Centre for Biotechnology Information (NCBI) which is accessible at http://www.ncbi.nlm.nih.gov/. Following identification of proteins through spectral-spectral correlation using the SEQUEST program, masses detected in MALDI-TOF mass spectra were assigned to tryptic digest peptides within the proteins identified. In cases where no proteins could be identified through searching with uninterpreted MS/MS spectra of tryptic digest peptides using the SEQUEST program, tandem mass spectra of the peptides were interpreted manually, using methods known in the art. (In the case of interpretation of low-energy fragmentation mass spectra of peptide ions see Gaskell et al., 1992, Rapid Commun. Mass Spectrom. 6:658-662).

Results

These initial experiments identified (a) 8 features that were decreased and 59 features that were increased in the liver tissue of subjects having early response as compared with the tissue of subjects free from liver response; (b) 10 features that were decreased and 67 features that were increased in the liver tissue of subjects having full manifestation response as compared with the tissue of subjects free from liver response; (c) 4 features that were decreased and 54 features that were increased in the liver tissue of subjects having late response as compared with the tissue of subjects free from liver response; (d) 82 features that were decreased and 42 features that were increased in the serum/plasma of subjects having early response as compared with the serum/plasma of subjects free from liver response; (e) 74 features that were decreased and 48 features that were increased in the serum/plasma of subjects having full manifestation liver response as compared with the serum/plasma of subjects free from liver response; (f) 47 features that were decreased and 37 features that were increased in the serum/plasma of subjects having late liver response as compared with the serum/plasma of subjects free from liver response. Overall, 17 features were identified as decreased and 155 features were identified as increased in the liver tissue of subjects with a liver response as compared with the tissue of subjects free from liver response, and 203 features were identified as decreased and 127 features were identified as increased in the blood of subjects with liver response as compared with subjects free from liver response. Details of these LRFs are provided in Tables I-XII. Each LRF was differentially present in the blood or liver of subjects having liver response as compared with the blood or liver of subjects free from liver response. For some preferred LRFs difference was highly significant (p<0.01) (LRF-1, LRF-2, LRF-3, LRF-4, LRF-5, LRF-386, LRF-387, LRF-388, LRF-20, LRF-24, LRF-36, LRF-40, LRF-41, LRF-44, LRF-51, LRF-390, LRF-396, LRF-53, LRF-54, LRF-56, LRF-57, LRF-400, LRF-64, LRF-96, LRF-97, LRF-99, LRF-100, LRF-101, LRF-390, LRF-396, LRF-404, LRF-107, LRF-108, LRF-112, LRF-115, LRF-134, LRF-139, LRF-142, LRF-143, LRF-144, LRF-145, LRF-404, LRF-160, LRF-161, LRF-165, LRF-169, LRF-178, LRF-181, LRF-185, LRF-189, LRF-192, LRF-193, LRF-194, LRF-195, LRF-196, LRF-197, LRF-198, LRF-199, LRF-200, LRF-201, LRF-202, LRF-203, LRF-204, LRF-235, LRF-433 and LRF-441 as markers of early liver response), LRF-260, LRF-263, LRF-268, LRF-269, LRF-273, LRF-274, LRF-275, LRF-276, LRF-277, LRF-288, LRF-289, LRF-290, LRF-297, LRF-298, LRF-300, LRF-301, LRF-302, LRF-305, LRF-308, LRF-309, LRF-310, LRF-311, LRF-312, LRF-313, LRF-319, LRF-320, LRF-431, LRF-434, LRF-441, LRF-447 and LRF-467 (as markers of full manifestation liver response), and LRF-472, LRF-473, LRF-474, LRF-328, LRF-329, LRF-340, LRF-341, LRF-342, LRF-353, LRF-425, LRF-426 , LRF-427, LRF-428, LRF-429, LRF-431, LRF-435, LRF-447,LRF-366, LRF-367, LRF-369, LRF-374, LRF-377, LRF-378, LRF-379, LRF-380 and LRF-381(as markers of late liver response.)) the, For certain highly preferred LRFs (LRF-434, LRF-425, and LRF-431), the difference was still more significant (p<0.001) .

Partial amino acid sequences were determined for the differentially present LRPIs in these LRFs. Details of these LRPIs are provided in Tables XIII to XXIV.

### EXAMPLES

Monitoring of Liver Damage

Identification of Hepatocellular Necrosis Markers Induced by Acetaminophen

Introduction:

Acetaminophen, used clinically as an analgesic drug, has been associated with hepatocellular necrosis in several species when administered at high doses. In this example, acetaminophen was evaluated to assess the potential toxicity observed in rats receiving a single oral gavage dose of 1000 mg/kg for one day. Animals were then necropsied at one of three time points corresponding to expectations of either early or full manifestation liver injury or recovery from acute liver injury/late liver response. The dose level and duration of exposure were based on a previous study reported in the literature in which a similar large dose of acetaminophen produced maximal hepatic necrosis accompanied by altered clinical chemistry parameters 12-18 hours after drug administration (Buttar, H. S. et al (1976) Toxicology 6(1): 9-20). Serum/plasma samples and portions of frozen liver were processed at room temperature, the resulting sera or liver supernatants were frozen at −80° C. awaiting 2-dimensional electrophoretic separation.

Methods:

Male Sprague-Dawley rats [Crl:CD (SD)BR] obtained from Charles River laboratories were distributed into 3 treatment groups and 3 vehicle control groups (5/sex/group) of similar weight distribution using a computer-based randomization procedure. Treated groups received a single dose of acetaminophen by oral intubation (20 ml/kg). Control animals received an equivalent dose volume of 0.5% methylcellulose (in deionized water) vehicle on all dosing days.

Compound was prepared on the day of dosing by weighing out the appropriate amount of acetaminophen and then transferring it into a mortar where it was mixed by pestle with vehicle until it could be transferred to beaker and diluted to the final desired volume. Vehicle consisted of methylcellulose prepared in deionized water at a final concentration of 0.5% (w/v). The drug preparation was stirred with a magnetic stir bar during the final dilution step and also during the dosing procedure. The final dosing volume was 20 ml/kg body weight.

Clinical observations were recorded once daily prior to treatment initiation and then at least twice daily during treatment. Body weights were obtained twice pre-study, daily during treatment, and then again prior to necropsy. Clinical pathology serum/plasma samples were collected via the vena cava at the time of necropsy for the measurement of clinical pathology parameters (hematology and serum/plasma chemistry). All animals were sedated prior to serum/plasma collection using an anesthetic mixture of 70% C0₂: 30% 0₂ gas.

Animals were necropsied, 5 treated and 5 controls each, at either 8 hours, 18 hours, or 72 hours post-dose. After gross examination, standard disposition procedures for the preservation of the liver, testes, lung, and brain from all animals for histopathological examination. After preserving specimens for histopathological examination and electron microscopy, a portion of the medial liver lobe, approximately 2 grams per animal, was snap-frozen and later processed for proteomic analysis.

Results:

(a) Clinical Observations:

All rats receiving acetaminophen survived the one-day dosing period. Acetaminophen was generally well tolerated with no adverse clinical signs observed in any of the control or treated groups.

(b) Body Weight:

Significant treatment related decreases in group mean body weights were observed at the second interim and final necropsies.

(c) Clinical Laboratory Measurements:

Statistically significant changes in the group means for several clinical chemistry markers were noted compared to concurrent vehicle controls. These are summarized as follows. Alanine aminotransferase increased 5.5× at 18 hr; 5′nucleotidase increased by 1.2× at 8 hrs; albumin increased by 1.1× at 18 hr; cholesterol increased by 1.1× at 72 hr; and glucose increased by 1.3× at 8 hr. Similarly, statistically significant changes in the group means for some hematology parameters were noted in drug-treated animals compared to vehicle controls as follows. Hematocrit, hemoglobin, and lymphocytes were increased 1.1×, 1.1×, and 1.8×, resp., at 8 hr; lymphocytes increased by 1.8× at 8 hr; mean cell hemoglobin increased by 1.1× at 18 hr; monocytes increased by 2.3× and 2.4×, resp., at 8 and 18 hr; neutrophils increased by 2.7× at 18 hr; % reticulocytes decreased by 2× at 72 hr; and white cell counts decreased by 1.7× at 8 hr.

(d) Gross Pathology Observations:

There were no treatment-related gross findings in rats given acetaminophen.

(e) Organ Weights:

There were no direct treatment-related changes in liver weights. At the second interim necropsy (18 hours post dose), mean relative liver weight of treated rats was statistically significantly increased (about 1.08× control mean) but this slight difference was attributed to decreased terminal body weight. Mean absolute liver weights were not affected by treatment at any time point.

(f) Microscopic Observations:

The principal treatment-related histopathologic finding was centrilobular necrosis evident in 5 of 5 rats at 18 hours post dose. In addition to coagulative necrosis, centrilobular necrosis also included mixed inflammatory cell infiltration, congestion, hemorrhage, and/or single cell necrosis. Slightly increased numbers of hepatocytes with mitotic figures were evident in controls, as compared to treated rats. The relevance of this finding is unknown since occasional hepatocyte mitoses are not uncommon in laboratory rodents. Many control livers also had slight focal or multifocal infiltration of mononuclear cells. Mononuclear cell infiltration of the liver is a common spontaneous background change in laboratory rodents (Greaves, 1990).

(g) Proteome Observations:

Using the reference protocol, proteins in liver tissue from animals having liver response (acetaminophen-treated animals) and control subjects were separated by isoelectric focusing followed by 2-D gel electrophoresis and analyzed by mass spectrometry as described in Section 6.1.14.

Table XXX. LRFs Identified in Liver Tissue of Subjects Having Acetaminophen-Treated Liver Response

TABLE XXX

| | Fold | | Mol Wt | P Value | |
|---|---|---|---|---|---|
| LRF# | Change | pI | (Da) | Rank Sum | Time |
| LRF-9 | 1.40 | 4.79 | 37664 | 0.0196 | Early |
| LRF-18 | 5.25 | 5.46 | 40000 | 0.0254 | Early |
| LRF-19 | 1.48 | 5.83 | 22631 | 0.0119 | Early |
| LRF-32 | 1.47 | 5.63 | 77203 | 0.0119 | Early |
| LRF-36 | 1.33 | 5.73 | 74823 | 0.0097 | Early |
| LRF-76 | 1.61 | 5.24 | 52175 | 0.0153 | Middle |
| LRF-88 | 1.50 | 6.21 | 23277 | 0.0122 | Middle |
| LRF-89 | 1.43 | 6.32 | 40144 | 0.0157 | Middle |
| LRF-107 | −1.25 | 4.66 | 13804 | 0.0097 | Late |
| LRF-110 | 2.33 | 6.19 | 52175 | 0.0216 | Late |
| LRF-114 | 1.45 | 6.10 | 183101 | 0.0119 | Late |
| LRF-130 | 1.36 | 6.21 | 57505 | 0.0278 | Late |
| LRF-400 | −17.27 | 8.84 | 11701 | 0.0075 | Middle, Late |
| LRF-405 | 1.40 | 6.76 | 30546 | 0.0278 | Middle, Late |

Table XXXI. LRFs Identified in Serum/Plasma of Subjects Having Acetaminophen-Treated Liver Response

TABLE XXXI

| | Fold | | Mol Wt | P Value | |
|---|---|---|---|---|---|
| LRF# | Change | pI | (Da) | Rank Sum | Time |
| LRF-159 | −1.51 | 6.09 | 138959 | 0.0122 | Early |
| LRF-166 | −3.22 | 5.65 | 38289 | 0.0122 | Early |
| LRF-170 | −1.90 | 6.03 | 103852 | 0.0122 | Early |
| LRF-174 | −1.78 | 6.37 | 73744 | 0.0122 | Early |
| LRF-176 | −1.25 | 4.62 | 51617 | 0.0160 | Early |
| LRF-177 | −1.53 | 7.08 | 19005 | 0.0160 | Early |
| LRF-178 | −1.38 | 7.54 | 104678 | 0.0097 | Early |
| LRF-219 | 1.42 | 4.95 | 34894 | 0.0122 | Early |
| LRF-221 | 2.07 | 5.29 | 38416 | 0.0122 | Early |
| LRF-222 | 1.45 | 4.8 | 37589 | 0.0122 | Early |
| LRF-232 | 1.56 | 4.72 | 65573 | 0.0122 | Early |
| LRF-234 | 1.49 | 5.11 | 30589 | 0.0122 | Early |
| LRF-237 | 2.08 | 5.45 | 49032 | 0.0122 | Early |
| LRF-258 | −2.15 | 5.61 | 35868 | 0.0122 | Full |
| | | | | | manifestation |
| LRF-261 | −1.63 | 5.03 | 41400 | 0.0122 | Full |
| | | | | | manifestation |
| LRF-264 | −1.59 | 6.78 | 53959 | 0.0122 | Full |
| | | | | | manifestation |
| LRF-265 | −4.20 | 6.83 | 28900 | 0.0073 | Full |
| | | | | | manifestation |
| LRF-266 | −1.98 | 5.38 | 38357 | 0.0122 | Full |
| | | | | | manifestation |
| LRF-270 | −1.82 | 5.57 | 15056 | 0.0122 | Full |
| | | | | | manifestation |
| LRF-271 | −1.33 | 4.67 | 51056 | 0.0122 | Full |
| | | | | | manifestation |
| LRF-297 | 5.28 | 6.8 | 39504 | 0.0075 | Full |
| | | | | | manifestation |
| LRF-303 | 1.87 | 4.9 | 75356 | 0.0122 | Full |
| | | | | | manifestation |
| LRF-306 | 1.35 | 6.67 | 28449 | 0.0117 | Full |
| | | | | | manifestation |
| LRF-356 | 5.22 | 6.13 | 29252 | 0.0108 | Late |
| LRF-370 | 4.13 | 5.32 | 25447 | 0.0108 | Late |
| LRF-372 | 3.78 | 6.39 | 85568 | 0.0108 | Late |

References:

Bachmann, E., Weber, E., Post, M., & Zibinden, G., Pharmacology 17: 39-49, 1978.

Buttar, H. S., Nera, E. A., & Downie, R. H., Toxicology 6: 9-20, 1976.

Greaves, P. (1990) Histopathology of Preclinical Toxicity Studies, p 414, Elsevier, Amsterdam

Laskin, D. L. & Pilaro, A. M., Toxicology & Applied Pharmacology 86:204-215, 1986.

Identification of Bile Duct Response Markers Induced by ANIT

Introduction:

Alpha-naphthylisothiocyanate (ANIT), a hepatotoxic agent associated with bile duct response. In this example, ANIT was evaluated to assess and compare overt liver toxicity with altered protein expression in the liver and serum/plasma of rats receiving a single oral gavage dose of 100 mg/kg/day and then necropsied at one of three time points. These times corresponded to expectations of either early or full manifestation liver injury or recovery from acute liver injury. The dose level and duration of exposure were based on previous studies reported in the literature in which hepatic or hepatobiliary effects were noted (Chisolm, J. W. and Dolphin, P. J. (1996) J. Lipid Res.37(5):1086-98). Serum/plasma samples and portions of frozen liver were processed at room temperature, the resulting sera or liver supernatants were frozen at −80° C. awaiting 2-dimensional electrophoretic separation.

Methods:

Male Sprague-Dawley rats [Crl:CD (SD)BR] obtained from Charles River Laboratories were distributed into 3 necropsy groups and three concurrent vehicle control groups (5/group) of similar weight distribution using a computer-based randomization procedure. Treated groups received ANIT as one single dose by oral gavage (10 ml/kg) at 100 mg/kg/day. Control animals received an equivalent dose volume of corn oil vehicle on the day of dosing.

Compound was prepared daily by weighing out the appropriate amount of ANIT and then transferring it to a pre-calibrated beaker. After a small amount of corn oil vehicle was added to wet the compound, the mixture was stirred to form a paste. Then the remainder of the total final volume of vehicle, 25 ml, was added to the beaker. The suspension was sonicated with a probe sonicator to disperse any remaining particles, using care not to overheat the preparation. The resulting clear golden solution was stirred prior to and during dosing with a magnetic stir bar and stir plate to enhance homogeneity.

Clinical observations were recorded once daily prior to treatment initiation and then at least twice daily on the day of dosing. Body weights were obtained twice pre-study and then prior to necropsy. Clinical pathology serum/plasma samples were collected via the vena cava at the time of necropsy for the measurement of clinical pathology parameters (hematology and serum/plasma chemistry). All animals were sedated prior to serum/plasma collection using an anesthetic mixture of 70% C0₂: 30% 0₂ gas.

Animals were necropsied, 5 treated and 5 controls each time, at either 12 hours, 48 hours, or 120 hours post-dose. After gross examination, standard disposition procedures for the preservation of the liver, testes, lung, and brain from all animals for definite histopathological examination (liver) or possible examination (all others) were followed. After gross examination, standard disposition procedures for the preservation of the liver, testes, lung, and brain from all animals for histopathological examination. After preserving specimens for histopathological examination and electron microscopy, a portion of the medial liver lobe, approximately 2 grams per animal, was snap-frozen and later processed for proteomic analysis.

Results

(a) Clinical Observations:

All rats receiving ANIT survived the 12, 48, or 120 hour post-dosing period. ANIT was generally well tolerated at all dose levels.

(b) Body Weight:

Group mean absolute and/or relative liver weights were significantly increased in treated rats compared to controls at the second interim and final necropsies. Group mean body weights were significantly decreased in treated rats compared to control animals at the first interim and final necropsies.

(c) Clinical Laboratory Measurements:

Numerous serum/plasma markers and hematology parameters were significantly altered by drug treatment, especially at 48 hours post-dose. These changes are summarized as follows. Alanine aminotransferase: increased by 2×, increased by 32× (12, 48 hr); aspartate aminotransferase: increased by 29× (48 hr); alkaline phosphatase decreased by 1.3×, decreased by 3.7×, decreased by 1.5× (12, 48, 120 hr); 5′ nucleotidase increased by 1.2×, increased by 1.3×, increased by 1.8× (12, 48, 120 hr); total bilirubin: increased by 104×, increased by 6× (48, 120 hr); bile acids increased by 54× (48 hr); total protein increased by 1.1× (12 hr); albumin decreased by 1.1× (48 hr); globulin increased by 1.2×, increased by 1.3×, increased by 1.2× (12,48, 120 hr); cholesterol increased by 5.5× (48 hr); glucose increased by 1.7× (12 hr); creatinine increased by 1.7× (48 hr); potassium increased by 1.2× (12,48, and 120 hr); calcium increased by 1.1× (12 hr); white serum/plasma cell count increased by 1.7×, increased by 1.6× (48, 120 hr); neutrophils increased by 4.4×, increased by 2.6× (48, 120 hr); lymphocytes decreased by 1.4×, increased by 1.4×, increased by 1.6× (12, 48, 120 hr); basophils decreased by 1.3× (12 hr); red serum/plasma cell count increased by 1.1× (12 hr); hemoglobin increased by 1.1× (12 hr); hematocrit increased by 1.1×, decreased by 1.1× (12, 120 hr); mean cell volume decreased by 1.1× (48 hr); platelets increased by 1.2×, increased by 1.3× (48, 120 hr), and fibrinogen increased by 1.8× (48 hr).

(d) Gross pathology Observations:

There were no treatment-related gross findings in rats given ANIT.

(e) Organ Weights:

Group mean absolute and/or relative liver weights were statistically significantly increased in treated rats compared to controls at the second interim and final necropsies. The increased weights were greatest at the second interim necropsy (about 1.1to 1.2× control mean at 48 hours post dose); at the final necropsy (120 hours post dose) absolute weight was similar to control mean, but relative liver weight remained increased due to decreased terminal body weights.

(f) Microscopic Observations:

Twelve hours post dose, one treated rat had slight multifocal single cell hepatocyte necrosis and another treated rat had pericholangial inflammation (two portal areas within the sections contained scattered inflammatory cells and were edematous). At 48 hours post dose, 4 of 5 treated rats had single cell hepatocyte necrosis, and all five treated rats had pericholangial inflammation. At this time point, the inflammatory change was qualitatively similar to that seen at the earlier time point (acute inflammation), but the severity was increased (increased inflammatory cells with edema, and greater number of pericholangial areas affected). At 120 hours post dose, single cell hepatocyte necrosis was absent but pericholangial inflammation persisted (5 of 5 affected). At this time point, pericholangial inflammation appeared more subacute (mixed inflammatory cells with more mononuclear cells) but the inflammation had diminished in severity (less edematous, fewer areas affected).

Almost all control rats had slight multifocal infiltration of mononuclear cells in the liver. Mononuclear cell infiltration of the liver is a common spontaneous background change in laboratory rodents (Greaves, 1990). The incidence and severity among treated rats was essentially similar, except at 120 hours post-dose when only 1 of 5 treated rats had mononuclear cell infiltration. However, at the 48-hour necropsy, mononuclear cell infiltration also included increased Kupffer cells, suggesting activation of the mononuclear cell phagocyte system by ANIT.

(g) Proteome Observations:

Using the reference protocol, proteins in liver tissue from subjects having liver response (ANIT induced) and control subjects were separated by isoelectric focusing followed by 2-D gel electrophoresis and analyzed by mass spectrometry as described in Section 6.1.14.

Table XXXII. LRFs Identified in Liver Tissue of Subjects Having ANIT induced Liver Response

TABLE XXXII
LRFs Identified in Liver Tissue of Subjects Having
ANIT induced Liver Response

| | Fold | | Mol Wt | P Value | |
|---|---|---|---|---|---|
| LRF# | Change | pI | (Da) | Rank Sum | Time |
| LRF-6 | 1.77 | 8.36 | 31027 | 0.0122 | Early |
| LRF-11 | 1.53 | 6.25 | 79681 | 0.0119 | Early |
| LRF-16 | 1.45 | 5.81 | 30167 | 0.0122 | Early |
| LRF-25 | 1.43 | 4.80 | 26633 | 0.0122 | Early |
| LRF-397 | 1.89 | 5.74 | 68828 | 0.0122 | Early, Middle |
| LRF-399 | 1.82 | 5.83 | 67405 | 0.0367 | Early, Middle |
| LRF-64 | 5.37 | 5.87 | 58635 | 0.0075 | Middle |
| LRF-67 | 3.32 | 4.90 | 33525 | 0.0119 | Middle |
| LRF-72 | 1.90 | 4.69 | 54509 | 0.0117 | Middle |
| LRF-81 | 1.57 | 6.61 | 41576 | 0.0119 | Middle |
| LRF-84 | 1.57 | 9.26 | 52497 | 0.0119 | Middle |
| LRF-409 | 1.29 | 5.59 | 27762 | 0.0119 | Middle, Late |
| LRF-410 | 1.31 | 10.23 | 38026 | 0.0345 | Middle, Late |
| LRF-111 | 1.41 | 5.57 | 23697 | 0.0200 | Late |
| LRF-123 | 1.73 | 4.63 | 11809 | 0.0119 | Late |
| LRF-124 | 1.71 | 6.27 | 27026 | 0.0122 | Late |
| LRF-127 | 1.89 | 5.02 | 49336 | 0.0122 | Late |
| LRF-133 | 1.60 | 6.30 | 27762 | 0.0122 | Late |

Table XXXIII. LRFs Identified in Serum/Plasma of Subjects Having ANIT induced Liver Response

TABLE XXXIII
LRFs Identified in Serum/Plasma of Subjects Having
ANIT induced Liver Response

| | | Fold | | Mol Wt | P Value | |
|---|---|---|---|---|---|---|
| LRF# | | Change | pI | (Da) | Rank Sum | Time |
| | LRF-157 | −1.28 | 6.17 | 28245 | 0.0122 | Early |
| | LRF-160 | −2.62 | 5.09 | 42125 | 0.0097 | Early |
| | LRF-161 | −4.38 | 4.94 | 36692 | 0.0075 | Early |
| | LRF-167 | −1.76 | 4.89 | 49621 | 0.0122 | Early |
| | LRF-169 | −8.33 | 5.62 | 45452 | 0.0075 | Early |
| | LRF-173 | −1.83 | 6.38 | 68285 | 0.0119 | Early |
| | LRF-181 | −6.05 | 6.06 | 43714 | 0.0075 | Early |
| | LRF-184 | −1.96 | 7.55 | 77168 | 0.0122 | Early |
| | LRF-218 | 1.35 | 5.42 | 23154 | 0.0122 | Early |
| | LRF-227 | 1.42 | 5.19 | 82775 | 0.0119 | Early |
| | LRF-230 | 1.78 | 5.13 | 121204 | 0.0119 | Early |
| | LRF-231 | 1.79 | 5.2 | 120973 | 0.0122 | Early |
| | LRF-298 | 6.40 | 7.08 | 39510 | 0.0075 | Middle |
| | LRF-305 | 6.03 | 6.2 | 33596 | 0.0073 | Middle |
| | LRF-367 | 6.89 | 4.78 | 49587 | 0.0073 | Late |

References:

Chisholm, J. W. & Dolphin, P. J., Journal of Lipid Research 37: 1086-1098, 1996.

Greaves, P. 1990, Histopathology of Preclinical Toxicity Studies, p 414, Elsevier, Amsterdam.

Quayle, J. A. et al., Clinical Science 75: 13-20, 1988.

Identification of Hepatomegaly Markers Induced by Phenobarbital

Introduction:

Phenobarbital, an anticonvulsant and sedative, has been associated with hepatomegaly in several species when administered at high doses. In this example, phenobarbital was evaluated to assess the potential liver effects observed in rats receiving oral administration by intubation of 100 mg/kg (s.i.d.) for up to four days. Animals were then necropsied at one of three time points corresponding to expectations of either early or full manifestation liver effects or recovery from these liver effects after cessation of dosing. The dose level and duration of exposure were based on previous studies reported in the literature in which a similar dosing regimen with phenobarbital produced hepatocellular hypertrophy and proliferation of smooth endoplasmic reticulum leading to liver enlargement (Crampton, R. F. et al (1997) Toxicology 7(3): 289-306). Serum/plasma samples and portions of frozen liver were processed at room temperature, the resulting sera or liver supernatants were frozen at −80° C. awaiting 2-dimensional electrophoretic separation.

Methods:

Male Sprague-Dawley rats [Crl:CD (SD)BR] obtained from Charles River Laboratories were distributed into 3 treatment groups and 3 vehicle control groups (5/sex/group) of similar weight distribution using a computer-based randomization procedure. Treated groups received up to three daily doses of phenobarbital, 100 mg/kg/day, by oral gavage (10 ml/kg). Control animals received an equivalent dose volume of deionized water vehicle on all dosing days.

Compound was prepared on the day of dosing by weighing out the appropriate amount of phenobarbital. This was transferred to a glass vessel and the total final volume of deionized water vehicle was added. Following agitation on a vortex mixture, a clear colorless solution was obtained. The final dosing volume was 10 ml/kg body weight.

Clinical observations were recorded once daily prior to treatment initiation and then at least twice daily during treatment. Body weights were obtained twice pre-study, daily during treatment, and then again prior to necropsy. Clinical pathology serum/plasma samples were collected via the vena cava at the time of necropsy for the measurement of clinical pathology parameters (hematology and serum/plasma chemistry). All animals were sedated prior to serum/plasma collection using an anesthetic mixture of 70% C0₂: 30% 0₂ gas.

Animals were necropsied, 5 treated and 5 controls each time, at 2, 5, or 12 days after the initiation of dosing. After gross examination, standard disposition procedures for the preservation of the liver, testes, lung, and brain from all animals for histopathological examination. After preserving specimens for histopathological examination and electron microscopy, a portion of the medial liver lobe, approximately 2 grams per animal, was snap-frozen and later processed for proteomic analysis.

Results

(a) Clinical Observation:

All rats receiving phenobarbital survived the one-day dosing period. Phenobarbital was generally well tolerated with no adverse clinical signs observed in any of the control or treated groups other than decreased activity.

(b) Body Weight:

No significant treatment-related changes in group mean body weights were observed at either interim necropsy when comparisons were made to concurrent control animals; however, at the final necropsy, a significant decrease in mean body weight was noted in the treated animals.

(c) Clinical Laboratory Measurements:

Statistically significant changes in the group means were noted for several clinical chemistry markers when compared to concurrent vehicle controls. These are summarized as follows. Total protein decreased by 1.1× on day 2; cholesterol increased by 1.5× on day 5; glucose decreased by 1.6× on day 5; serum/plasma urea nitrogen decreased by 1.8× on day 2 and increased by 1.2× on day 12; and creatinine decreased by 1.7× on day 2. Except for small but significant decreases in % reticulocytes on days 5 and 12, the group means for the various hematology parameters were not altered in drug-treated animals compared to vehicle control animals.

(d) Gross Pathology Observations:

There were no treatment-related gross findings in rats given phenobarbital.

(e) Organ Weights:

Group mean absolute and/or relative liver weights were statistically significantly increased in treated rats compared to controls at all time points. The increased weights were greatest at the second interim necropsy, and were approaching baseline at the final necropsy. Two days post dose, liver weights were increased about 1.1× control mean, whereas five days post dose, the increase was about 1.5× control mean. At the final necropsy (twelve days post dose), absolute liver weights were about the same as control, but relative liver weight was still increased about 1.06× control mean.

(f) Microscopic Observations:

Increased liver weights were attributable to centrilobular hypertrophy, which was evident in 5 of 5 treated rats two days post dose. Increased numbers of hepatocytes with either apoptotic bodies or mitotic figures accompanied centrilobular hypertrophy in 3 of 5 treated rats at the same time point. These histopathologic findings were not evident in livers obtained at either two or twelve days post dose.

(g) Proteome Observations:

Using the reference protocol, proteins in liver tissue from subjects having liver response (phenobarbital induced) and control subjects were separated by isoelectric focusing followed by 2-D gel electrophoresis and analyzed by mass spectrometry as described in Section 6.1.14.

Table XXXIV. LRFs Identified in Liver Tissue of Subjects Having Phenobarbital Induced Liver Response

TABLE XXXIV
LRFs Identified in Liver Tissue of Subjects Having
Phenobarbital Induced Liver Response

| | Fold | | Mol Wt | P Value | |
|---|---|---|---|---|---|
| LRF# | Change | pI | (Da) | Rank Sum | Time |
| LRF-17 | 1.61 | 5.81 | 30167 | 0.0212 | Early |
| LRF-24 | 3.34 | 10.76 | 23549 | 0.0075 | Early |
| LRF-26 | 1.33 | 5.28 | 58041 | 0.0216 | Early |
| LRF-37 | 3.98 | 7.59 | 33242 | 0.0254 | Early |
| LRF-60 | 1.67 | 6.80 | 37664 | 0.0216 | Middle |
| LRF-66 | 1.93 | 5.44 | 24451 | 0.0345 | Middle |
| LRF-74 | 1.42 | 6.70 | 92196 | 0.0361 | Middle |
| LRF-80 | 1.40 | 5.12 | 127612 | 0.0216 | Middle |
| LRF-83 | 1.40 | 5.00 | 15334 | 0.0367 | Middle |
| LRF-85 | 1.72 | 6.29 | 128480 | 0.0122 | Middle |
| LRF-90 | 1.47 | 5.14 | 106705 | 0.0216 | Middle |
| LRF-112 | 5.38 | 4.99 | 49643 | 0.0075 | Late |
| LRF-115 | 3.16 | 5.85 | 162581 | 0.0075 | Late |

Table XXXV. LRFs Identified in Serum/Plasma of Subjects Having Phenobarbital Induced Liver Response

TABLE XXXV
LRFs Identified in Serum/Plasma of Subjects Having
Phenobarbital Induced Liver Response

| | Fold | | Mol Wt | P Value | |
|---|---|---|---|---|---|
| LRF# | Change | pI | (Da) | Rank Sum | Time |
| LRF-158 | −1.33 | 5.89 | 128039 | 0.0119 | Early |
| LRF-179 | −1.37 | 6.84 | 108902 | 0.0212 | Early |
| LRF-182 | −2.81 | 5.76 | 68203 | 0.0178 | Early |
| LRF-217 | 2.16 | 5.06 | 44232 | 0.0122 | Early |
| LRF-223 | 1.34 | 7.43 | 28246 | 0.0117 | Early |
| LRF-224 | 1.30 | 7.14 | 28269 | 0.0216 | Early |
| LRF-235 | 8.26 | 6.9 | 88853 | 0.0075 | Early |
| LRF-257 | −1.42 | 5.69 | 35827 | 0.0122 | Middle |
| LRF-259 | −1.34 | 5.32 | 23373 | 0.0212 | Middle |
| LRF-294 | 1.57 | 4.95 | 32192 | 0.0216 | Middle |
| LRF-369 | 2.53 | 5.25 | 19817 | 0.0097 | Late |
| LRF-371 | 3.02 | 4.85 | 112218 | 0.0109 | Late |

References:

Crampton, R. F et al.,Toxicology 7:289-306, 1977.

Johnson, S. et al., Human & Experimental Toxicology 12: 153-158, 1993.

Leeson, G. A. et al., Fundamental & Applied Toxicology 4: 261-269, 1984.

Identification of Hepatomegaly Markers Induced by Wy-14,643

Introduction:

Wy-14,643, a peroxisome proliferating agent in rodents, has been associated with hepatomegaly in several species when administered at high doses. In this example, Wy-14,643 was evaluated to assess the potential toxicity observed in rats receiving subcutaneous administration of 45 mg/kg (s.i.d.) for up to three days. Animals were then necropsied at one of three time points corresponding to expectations of either early or full manifestation liver effects or recovery from these liver effects. The dose level and duration of exposure were based on a previous study reported in the literature in which a similar dosing regimen with Wy-14,643 produced hepatic cell proliferation leading to liver enlargement (Marsman, D. S. et al (1988) Cancer Res.48(23): 6739-44; Wada, N. et al (1992) Fund. Applied Toxicology 18(1):149-54) Serum/plasma samples and portions of frozen liver were processed at room temperature, the resulting sera or liver supernatants were frozen at −80° C. awaiting 2-dimensional electrophoretic separation.

Methods:

Male Sprague-Dawley rats [Crl: CD (SD)BR] obtained from Charles River Laboratories were distributed into 3 treatment groups and 3 vehicle control groups (5/sex/group) of similar weight distribution using a computer-based randomization procedure. Treated groups received up to three daily doses of Wy-14,643, 45mg/kg/day, by subcutaneous injection (0.2 ml/kg). Control animals received an equivalent dose volume of corn oil vehicle on all dosing days.

Compound was prepared on the day of dosing by weighing out the appropriate amount of Wy-14,643 into a glass vessel and adding the total volume of corn oil. The mixture was stirred using a magnetic stir bar and sonicated to break up visible particles producing a slightly cloudy, yellow suspension. The drug preparation was stirred with a magnetic stir bar during the dosing procedure to enhance homogeneity. The final dosing volume was 0.2 ml/kg body weight.

Clinical observations were recorded once daily prior to treatment initiation and then at least twice daily during treatment. Body weights were obtained twice pre-study, daily during treatment, and then again prior to necropsy. Clinical pathology serum/plasma samples were collected via the vena cava at the time of necropsy for the measurement of clinical pathology parameters (hematology and serum/plasma chemistry). All animals were sedated prior to serum/plasma collection using an anesthetic mixture of 70% C0₂: 30% 0₂ gas.

Animals were necropsied, 5 treated and 5 controls each time, at 2, 4, or 15 days after the initiation of dosing. After gross examination, standard disposition procedures for the preservation of the liver, testes, lung, and brain from all animals for histopathological examination. After preserving specimens for histopathological examination and electron microscopy, a portion of the medial liver lobe, approximately 2 grams per animal, was snap-frozen and later processed for proteomic analysis.

Results

(a) Clinical Observations:

Rats receiving Wy-14,643 survived the one-day dosing period. Wy-14,643 was generally well tolerated with no adverse clinical signs observed in any of the control or treated groups.

(b) Body Weight:

Significant treatment-related changes in group mean body weights were observed at interim or final necropsies when comparisons were made to concurrent control animals.

(c) Clinical Laboratory Measurements:

Statistically significant changes in the group means were noted for several clinical chemistry markers when compared to concurrent vehicle controls. These are summarized as follows. Alanine aminotransferase increased by 1.2× at day 15; alkaline phosphatase increased by 1.2× at day 2; 5′nucleotidase decreased by 1.3×, 1.3×, and 1.2× on days 2, 4, and 15, resp.; total protein decreased by 1.1× on day 15; globulin decreased by 1.1× and 1.2× on days 2 and 4, resp., and increased by 1.1× on day 15; cholesterol decreased by 1.6× on day 2; and glucose decreased by 1.3× on day 2 and increased by 1.4× on day 15. Similarly, statistically significant changes in the group means for some hematology parameters were noted in drug-treated animals compared to vehicle controls as follows: White cell counts decreased by 1.3× on day 4; lymphocytes decreased by 1.3× on day 4; and % reticulocytes decreased by 1.2× on day 4.

(d) Gross Pathology Observations:

There were no treatment-related gross findings in rats given Wy-14,643.

(e) Organ Weights:

Group mean absolute and/or relative liver weights were increased in treated rats compared to controls at all time points, but not all increases were statistically significant. The increased weights were greatest at the second interim necropsy, and were approaching baseline at the final necropsy. Two days post dose, liver weights were increased about 1.1-1.2× control mean, whereas four days post dose, the increase was about 1.4× control mean. At the final necropsy (fourteen days post dose), absolute liver weights were about the same as control, but relative liver weight was still increased about 1.1× control mean.

(f) Microscopic Observations:

Two days post dose, the principal histopathologic change was increased number of hepatocytes with mitotic figures (4 of 5). Four days post dose, histopathologic changes were limited to centrilobular hypertrophy and karyomegaly in 5 of 5 treated rats. Increased liver weights were attributed to these changes. Hypertrophy and karyomegaly persisted at the fourteen-day necropsy; however, there was a reduction in severity of hypertrophy and in the incidence of karyomegaly. A few rats in both control and treated groups had slight multifocal infiltration of mononuclear cells in the liver. The incidence was essentially similar between control and treated groups. Mononuclear cell infiltration of the liver is a common spontaneous background change in laboratory rodents (Greaves, 1990).

(g) Proteome Observations:

Using the reference protocol, proteins in liver tissue from subjects having liver response (Wy-14,643 induced) and control subjects were separated by isoelectric focusing followed by 2-D gel electrophoresis and analyzed by mass spectrometry as described in Section 6.1.14.

Table XXXVI. LRFs Identified in Liver Tissue of Subjects Having Wy-14,643 Induced Liver Response

TABLE XXXVI
LRFs Identified in Liver Tissue of Subjects Having
Wy-14,463 Induced Liver Response

| | Fold | | Mol Wt | P Value | |
|---|---|---|---|---|---|
| LRF# | Change | pI | (Da) | Rank Sum | Time |
| LRF-1 | −4.87 | 8.95 | 16081 | 0.0075 | Early |
| LRF-2 | −1.42 | 4.60 | 62363 | 0.0088 | Early |
| LRF-3 | −5.87 | 6.59 | 12894 | 0.0075 | Early |
| LRF-4 | −4.37 | 4.93 | 26181 | 0.0073 | Early |
| LRF-10 | 2.13 | 7.73 | 123358 | 0.0122 | Early |
| LRF-15 | 2.47 | 9.38 | 80495 | 0.0122 | Early |
| LRF-21 | 1.50 | 8.25 | 35532 | 0.0122 | Early |
| LRF-23 | 1.90 | 8.06 | 31812 | 0.0122 | Early |
| LRF-29 | 1.57 | 6.19 | 77203 | 0.0122 | Early |
| LRF-31 | 2.07 | 8.97 | 27694 | 0.0119 | Early |
| LRF-53 | −5.83 | 6.14 | 72138 | 0.0075 | Middle |
| LRF-54 | −2.33 | 4.81 | 51921 | 0.0097 | Middle |
| LRF-61 | 3.09 | 8.08 | 14573 | 0.0119 | Middle |
| LRF-70 | 1.50 | 9.50 | 11592 | 0.0160 | Middle |
| LRF-78 | 1.95 | 6.36 | 32515 | 0.0122 | Middle |
| LRF-79 | 1.34 | 7.52 | 24017 | 0.0200 | Middle |
| LRF-91 | 1.18 | 6.21 | 39258 | 0.0117 | Middle |
| LRF-113 | 1.31 | 5.84 | 45749 | 0.0119 | Late |
| LRF-121 | 1.24 | 5.51 | 54245 | 0.0367 | Late |
| LRF-386 | −4.23 | 5.01 | 27830 | 0.0075 | Early, Middle |
| LRF-387 | −6.63 | 5.44 | 24451 | 0.0075 | Early, Middle |
| LRF-390 | 5.03 | 6.36 | 46232 | 0.0073 | Early, Middle |
| LRF-391 | 1.66 | 6.97 | 28172 | 0.0119 | Early, Middle |
| LRF-392 | 1.29 | 7.98 | 29700 | 0.0122 | Early, Middle |
| LRF-393 | 1.51 | 8.88 | 28876 | 0.0122 | Early, Middle |
| LRF-394 | 1.17 | 6.50 | 25990 | 0.0459 | Early, Middle |
| LRF-395 | 1.97 | 9.74 | 25549 | 0.0119 | Early, Middle |
| LRF-407 | 1.62 | 6.04 | 25363 | 0.0119 | Middle, Late |

Table XXXVII. LRFs Identified in Serum/Plasma of Subjects Having Wy-14,643 Induced Liver Response

TABLE XXXVII
LRFs Identified in Serum/Plasma of Subjects Having
Wy-14,643 Induced Liver Response

| | Fold | | Mol Wt | P Value | |
|---|---|---|---|---|---|
| LRF# | Change | pI | (Da) | Rank Sum | Time |
| LRF-162 | −2.1 | 4.95 | 35503 | 0.0122 | Early |
| LRF-163 | −2.29 | 5.13 | 31133 | 0.0122 | Early |
| LRF-165 | −4.04 | 5.88 | 27988 | 0.0075 | Early |
| LRF-228 | 1.36 | 6.3 | 73388 | 0.0449 | Early |
| LRF-256 | −1.39 | 5.06 | 38779 | 0.0119 | Middle |
| LRF-260 | −5.26 | 4.97 | 46213 | 0.0075 | Middle |
| LRF-263 | −1.61 | 5.39 | 62144 | 0.0119 | Middle |
| LRF-295 | 1.48 | 7.31 | 56943 | 0.0119 | Middle |
| LRF-300 | 1.97 | 5.25 | 91060 | 0.0097 | Middle |
| LRF-301 | 12.62 | 5.67 | 81660 | 0.0075 | Middle |
| LRF-329 | −3.14 | 5.94 | 33501 | 0.0075 | Late |
| LRF-332 | −2.69 | 5.85 | 61591 | 0.0122 | Late |
| LRF-358 | 1.22 | 5.33 | 36864 | 0.0122 | Late |
| LRF-360 | 2.14 | 4.86 | 36619 | 0.0122 | Late |
| LRF-361 | 1.67 | 5.07 | 31110 | 0.0119 | Late |
| LRF-373 | 1.74 | 5.63 | 12297 | 0.0122 | Late |

References:

Greaves, P. 1990, Histopathology of Preclinical Toxicity Studies, p 414, Elsevier, Amsterdam.

Marsman, D. S. et al., Cancer Research 48:6739-6744, 1988.

Wada, N. et al., Fundamental & Applied Toxicology 18: 149-154, 1992.

Xinfang, M. A. et al., Molecular Pharmacology 51: 69-78, 1997.

Identification of Steatosis Markers Induced by Valproic Acid

Introduction

Valproic acid is an anticonvulsant agent associated with fatty liver (steatosis) when administered at high doses. This drug was evaluated to compare liver response with altered protein expression in liver and serum/plasma in rats receiving a subcutaneous injection of 700 mg/kg/day s.i.d. for up 2 days and then necropsied at one of three time points. These times corresponded to expectations of either early or full manifestation liver injury or recovery from subacute liver injury. The dose level and duration of exposure were based on previous studies reported in the literature in which a similar dose of valproic acid consistently produced significant microvesicular steatosis in adult male rat liver after 48 hours (Kesterson, J. W. et al (1984) Hepatology 4(6): 1143-52; Lewis, J. H. et al (1982) Hepatology 2(6):870-3). Serum/plasma samples and portions of frozen liver were processed at room temperature, the resulting sera or liver supernatants were frozen at −80° C. awaiting 2-dimensional electrophoretic separation.

Methods

Male Sprague-Dawley rats [Crl:CD (SD)BR] obtained from Charles River Laboratories were distributed into 3 necropsy groups and three concurrent vehicle control groups (5/group) of similar weight distribution using a computer-based randomization procedure. Treated groups received valproic acid, 700 mg/kg/day, by subcutaneous injection in the mid-dorsal region (2 ml/kg) s.i.d. for up to two days. Control animals received an equivalent dose volume of sterile saline vehicle on the day of dosing.

Compound was prepared daily by placing the required amount of valproic acid in a graduated cylinder and slowly bringing to the desired final volume by adding sterile saline while mixing on a vortex mixer. The resulting clear colorless solution was again mixed on a vortex mixer prior to each dose withdrawal.

Clinical observations were recorded once daily prior to treatment initiation and then at least twice daily on the day of dosing. Body weights were obtained twice pre-study and then prior to necropsy. Clinical pathology blood samples were collected via the vena cava at the time of necropsy for the measurement of clinical pathology parameters (hematology and serum/plasma chemistry). All animals were sedated prior to blood collection using an anesthetic mixture of 70% C0₂: 30% 0₂ gas.

Animals were necropsied, 5 treated and 5 controls each time, at 2, 4, or 15 days after the initiation of dosing. After gross examination, standard disposition procedures for the preservation of the liver, testes, lung, and brain from all animals for histopathological examination. After preserving specimens for histopathological examination and electron microscopy, a portion of the medial liver lobe, approximately 2 grams per animal, was snap-frozen and later processed for proteomic analysis.

Results

(a) Clinical Observation

All rats receiving valproic acid survived the 2 hr, 3 day, or 8-day post-dosing period, respectively. Valproic acid was generally well tolerated at all dose levels. Clinical signs in treated groups were limited to decreased activity and some ataxia in front and hind limbs within an hour after treatment.

(b) Body Weight

Neither the group mean absolute or relative liver weights were significantly changed in treated rats compared to concurrent controls at any of the three necropsies. The group mean terminal body weight was significantly decreased in treated animals compared to controls at the final necropsy.

(c) Clinical Laboratory Measurements

Statistically significant changes in the group means for several clinical chemistry markers were noted compared to concurrent vehicle controls. These are summarized as follows. Alkaline phosphatase decreased by 1.3× at 3 days; 5′-nucleotidase decreased by 1.3× at 3 days; total protein decreased by 1.1× at 3 days; globulin decreased by 1.1× at 3 days; and glucose decreased by 2.3× at 2 hr but increased by 1.3× at 8 days. Similarly, statistically significant changes in the group means for some hematology parameters were noted in drug-treated animals compared to vehicle controls as follows. White blood cell counts decreased by 1.8× at 2 hr, lymphocytes decreased by 1.7× at 2 hr; and basophils decreased by 5.1× at 2 hr.

(d) Gross Pathology Observations

There were no treatment-related gross findings in rats given valproic acid.

(e) Organ Weights

There were no liver weight changes associated with treatment.

(f) Microscopic Observations

Histopathologic evidence of treatment-related hepatic steatosis was equivocal. Minimal hepatocyte vacuolation was observed in 3 of 15 treated rats (two at the 2-hour post dose necropsy, and one at the 3-day post dose necropsy). However, oil red-O staining for lipids failed to reveal positive stain retention in these livers greater than controls. Some rats in both control and treated groups had slight multifocal infiltration of mononuclear cells in the liver. The incidence was essentially similar between control and treated animals across all time points. Mononuclear cell infiltration of the liver is a common spontaneous background change in laboratory rodents (Greaves, 1990).

(g) Proteome Observations:

Using the reference protocol, proteins in liver tissue from subjects having liver response (valproic acid induced) and control subjects were separated by isoelectric focusing followed by 2-D gel electrophoresis and analyzed by mass spectrometry as described in Section 6.1.14.

Table XXXVIII. LRFs Identified in Liver Tissue of Subjects Having Valproic Acid Induced Liver Response

TABLE XXXVIII
LRFs Identified in Liver Tissue of Subjects Having
Valproic Acid Induced Liver Response

| | Fold | | Mol Wt | P Value | |
|---|---|---|---|---|---|
| LRF# | Change | pI | (Da) | Rank Sum | Time |
| LRF-8 | 1.25 | 5.57 | 23697 | 0.0122 | Early |
| LRF-14 | 2.01 | 4.99 | 56166 | 0.0200 | Early |
| LRF-22 | 1.41 | 9.80 | 11450 | 0.0122 | Early |
| LRF-27 | 1.22 | 7.31 | 124197 | 0.0114 | Early |
| LRF-28 | 1.53 | 5.65 | 49698 | 0.0119 | Early |
| LRF-30 | 2.08 | 4.73 | 57645 | 0.0112 | Early |
| LRF-38 | 2.09 | 5.02 | 56260 | 0.0216 | Early |
| LRF-56 | −3.20 | 6.05 | 45895 | 0.0073 | Middle |
| LRF-69 | 1.32 | 7.67 | 40789 | 0.0122 | Middle |
| LRF-71 | 1.35 | 7.80 | 27225 | 0.0122 | Middle |
| LRF-73 | 1.15 | 7.82 | 55445 | 0.0117 | Middle |
| LRF-75 | 1.36 | 7.58 | 30261 | 0.0200 | Middle |
| LRF-92 | 1.47 | 6.51 | 59855 | 0.0117 | Middle |
| LRF-109 | 1.33 | 8.13 | 64092 | 0.0216 | Late |
| LRF-119 | 1.68 | 6.86 | 46042 | 0.0212 | Late |
| LRF-120 | 1.33 | 8.25 | 35532 | 0.0216 | Late |
| LRF-131 | 1.10 | 5.16 | 12722 | 0.0200 | Late |
| LRF-132 | 1.25 | 6.30 | 66705 | 0.0216 | Late |
| LRF-389 | 1.15 | 9.76 | 18844 | 0.0465 | Early, Middle |
| LRF-401 | 1.30 | 7.13 | 39509 | 0.0119 | Middle, Late |
| LRF-402 | 1.19 | 7.37 | 51294 | 0.0122 | Middle, Late |
| LRF-403 | 1.55 | 7.83 | 51921 | 0.0367 | Middle, Late |

Table XXXIX. LRFs Identified in Serum/Plasma of Subjects Having Valproic Acid Induced Liver Response

| | Fold | | Mol Wt | P Value | |
|---|---|---|---|---|---|
| LRF# | Change | pI | (Da) | Rank Sum | Time |
| LRF-156 | −1.38 | 4.93 | 54483 | 0.0216 | Early |
| LRF-164 | −1.56 | 5.31 | 40165 | 0.0122 | Early |
| LRF-168 | −2.49 | 5.1 | 73017 | 0.0122 | Early |
| LRF-172 | −1.67 | 5.47 | 69633 | 0.0119 | Early |
| LRF-175 | −2.33 | 5.4 | 86193 | 0.0122 | Early |
| LRF-183 | −1.63 | 5.39 | 103579 | 0.0160 | Early |
| LRF-220 | 1.32 | 6.99 | 78836 | 0.0367 | Early |
| LRF-225 | 1.52 | 6.25 | 90963 | 0.0119 | Early |
| LRF-226 | 1.68 | 6.02 | 100636 | 0.0119 | Early |
| LRF-236 | 1.47 | 6.09 | 39155 | 0.0216 | Early |
| LRF-262 | −6.83 | 4.93 | 31680 | 0.0075 | Middle |
| LRF-296 | 1.50 | 7.21 | 28524 | 0.0117 | Middle |
| LRF-328 | −6.78 | 6.54 | 139228 | 0.0073 | Late |
| LRF-331 | −2.50 | 5.97 | 140431 | 0.0122 | Late |
| LRF-334 | −1.63 | 5.85 | 101552 | 0.0122 | Late |
| LRF-335 | −1.89 | 6.56 | 71935 | 0.0112 | Late |
| LRP-359 | 1.73 | 5.08 | 33850 | 0.0122 | Late |
| LRF-363 | 1.62 | 5.13 | 43976 | 0.0122 | Late |
| LRF-364 | 4.15 | 5.48 | 38709 | 0.0075 | Late |
| LRF-365 | 1.62 | 5.71 | 72892 | 0.0119 | Late |
| LRF-366 | 2.77 | 4.8 | 48783 | 0.0097 | Late |
| LRF-368 | 2.22 | 4.93 | 69794 | 0.0122 | Late |

Conclusions

Under this experimental regimen, valproic acid was expected to produce hepatic steatosis or fatty liver. Significant decreases in serum/plasma alkaline phosphatase, 5′ nucleotidase, total protein, and globulins were all noted at the midpoint. These changes were similar to the clinical chemistry effects previously reported in humans. Histopathologic evidence of treatment-related hepatic steatosis was equivocal. Minimal hepatocyte vacuolation was observed in 3 of 15 treated rats (two at the 2-hour post dose necropsy, and one at the 3-day post dose necropsy). However, oil red-O staining for lipids failed to reveal positive stain retention in these livers greater than controls.

References

Greaves, P. 1990, Histopathology of Preclinical Toxicity Studies, p 414, Elsevier, Amsterdam.

Kesterson, J. W. et al., Hepatology 4: 1143-1152, 1984.

Lewis, J. H. et al., Hepatology 2: 870-873, 1982.

Olson, M. J. et al., Molecular Pharmacology 30: 520-525, 1986.

The present invention is not to be limited in terms of the particular embodiments described in this application, which are intended as single illustrations of individual aspects of the invention. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The contents of each reference, patent and patent application cited in this application is hereby incorporated by reference in its entirety.

Other LRF's identified by the methods described herein include:

Table XL. LRFs Decreased in Liver Tissue of Subjects Having Early Liver Response

TABLE XL

| | Relative Abundance | | | |
|---|---|---|---|---|
| | (foreground over | | | P value (Rank Sum |
| LRF# | background) | pI | MW (Da) | Test) |
| LRF-5 | −3.50 | 5.64 | 17916 | 0.0075 |
| LRF-388 | −3.30 | 5.00 | 17359 | 0.0075 |

Table XLI. LRFs Increased in Liver Tissue of Subjects Having Early Liver Response

TABLE XLI

| | Relative Abundance | | | |
|---|---|---|---|---|
| | (foreground over | | | P value (Rank Sum |
| LRF# | background) | pI | MW (Da) | Test) |
| LRF-39 | 1.62 | 6.25 | 25425 | 0.0122 |
| LRF-40 | 2.94 | 5.03 | 63314 | 0.0073 |
| LRF-41 | 3.74 | 5.06 | 63314 | 0.0075 |
| LRF-42 | 1.41 | 6.71 | 17836 | 0.0112 |
| LRF-43 | 1.42 | 10.61 | 21622 | 0.0200 |
| LRF-44 | 4.64 | 10.28 | 43875 | 0.0075 |
| LRF-45 | 1.36 | 6.53 | 26858 | 0.0119 |
| LRF-46 | 1.88 | 5.69 | 37544 | 0.0119 |
| LRF-47 | 1.67 | 4.80 | 26633 | 0.0160 |
| LRF-48 | 2.63 | 5.03 | 59855 | 0.0232 |
| LRF-49 | 1.65 | 7.40 | 102341 | 0.0254 |
| LRF-50 | 2.35 | 6.80 | 96062 | 0.0254 |
| LRF-51 | 3.72 | 10.44 | 134677 | 0.0097 |
| LRF-52 | 1.48 | 6.97 | 16674 | 0.0119 |
| LRF-396 | 13.36 | 5.62 | 12272 | 0.0075 |
| LRF-398 | 3.99 | 5.53 | 41466 | 0.0109 |

Table XLII. LRFs Decreased in Liver Tissue of Subjects Having Full Manifestation Liver Response

TABLE XLII

| | Relative Abundance | | | |
|---|---|---|---|---|
| | (foreground over | | | P value (Rank Sum |
| LRF# | background) | pI | MW (Da) | Test) |
| LRF-57 | −7.32 | 8.89 | 12387 | 0.0073 |
| LRF-388 | −3.30 | 5.00 | 17359 | 0.0075 |

Table XLIII. LRFs Increased in Liver Tissue of Subjects Having Full Manifestation Liver Response

TABLE XLIII

| | Relative Abundance | | | |
|---|---|---|---|---|
| | (foreground over | | | P value (Rank Sum |
| LRF# | background) | pI | MW (Da) | Test) |
| LRF-60 | 1.67 | 6.80 | 37664 | 0.0216 |
| LRF-93 | 1.52 | 10.47 | 86339 | 0.0119 |
| LRF-94 | 1.99 | 4.86 | 46931 | 0.0119 |
| LRF-95 | 1.23 | 4.95 | 32011 | 0.0196 |
| LRF-96 | 5.95 | 6.31 | 29353 | 0.0075 |
| LRF-97 | 4.27 | 5.31 | 23069 | 0.0075 |
| LRF-98 | 1.63 | 6.14 | 45458 | 0.0129 |
| LRF-99 | 3.06 | 5.25 | 90962 | 0.0073 |
| LRF-100 | 5.20 | 4.82 | 67138 | 0.0075 |
| LRF-101 | 1.84 | 9.29 | 50674 | 0.0095 |
| LRF-102 | 1.32 | 5.30 | 92678 | 0.0119 |
| LRF-103 | 2.33 | 5.30 | 30633 | 0.0114 |
| LRF-104 | 1.72 | 6.17 | 26828 | 0.0119 |
| LRF-396 | 13.36 | 5.62 | 12272 | 0.0075 |
| LRF-398 | 3.99 | 5.53 | 41466 | 0.0109 |
| LRF-404 | 2.88 | 6.86 | 85252 | 0.0097 |
| LRF-406 | 2.73 | 8.50 | 61937 | 0.0112 |
| LRF-408 | 2.79 | 5.63 | 36671 | 0.0248 |
| LRF-411 | 1.14 | 9.50 | 32111 | 0.0465 |

Table XLIV. LRFs Decreased in Liver Tissue of Subjects Having Late Liver Response

TABLE XLIV

| | Relative Abundance | | | |
|---|---|---|---|---|
| | (foreground over | | | P value (Rank Sum |
| LRF# | background) | pI | MW (Da) | Test) |
| LRF-108 | −3.95 | 5.55 | 49336 | 0.0075 |

Table XLV. LRFs Increased in Liver Tissue of Subjects Having Late Liver Response

TABLE XLV

| | Relative Abundance | | | |
|---|---|---|---|---|
| | (foreground over | | | P value (Rank Sum |
| LRF# | background) | pI | MW (Da) | Test) |
| LRF-125 | 1.36 | 6.91 | 88425 | 0.0119 |
| LRF-135 | 1.53 | 5.45 | 27830 | 0.0119 |
| LRF-136 | 1.27 | 5.19 | 17179 | 0.0119 |
| LRF-137 | 1.27 | 7.76 | 20342 | 0.0122 |
| LRF-138 | 1.54 | 5.45 | 59149 | 0.0160 |
| LRF-139 | 5.90 | 5.72 | 14317 | 0.0075 |
| LRF-140 | 1.58 | 5.45 | 24942 | 0.0117 |
| LRF-141 | 1.49 | 5.69 | 13244 | 0.0117 |
| LRF-142 | 4.24 | 5.88 | 41260 | 0.0073 |
| LRF-143 | 2.68 | 5.82 | 19164 | 0.0097 |
| LRF-144 | 2.04 | 5.15 | 93651 | 0.0097 |
| LRF-145 | 2.67 | 5.78 | 27239 | 0.0071 |
| LRF-146 | 3.44 | 5.01 | 63802 | 0.0254 |
| LRF-147 | 1.39 | 5.25 | 92678 | 0.0367 |
| LRF-398 | 3.99 | 5.53 | 41466 | 0.0109 |
| LRF-404 | 2.88 | 6.86 | 85252 | 0.0097 |
| LRF-406 | 2.73 | 8.50 | 61937 | 0.0112 |
| LRF-408 | 2.79 | 5.63 | 36671 | 0.0248 |
| LRF-411 | 1.14 | 9.50 | 32111 | 0.0465 |

Table XLVI. LRFs Decreased in Blood of Subjects Having Early Liver Response

TABLE XLVI

| | Relative Abundance | | | |
|---|---|---|---|---|
| | (foreground over | | | P value (Rank Sum |
| LRF# | background) | pI | MW (Da) | Test) |
| LRF-180 | −1.39 | 5.71 | 108153 | 0.0278 |
| LRF-185 | −7.52 | 5.10 | 49435 | 0.0075 |
| LRF-186 | −1.24 | 5.15 | 35066 | 0.0178 |
| LRF-187 | −1.27 | 5.70 | 107249 | 0.0345 |
| LRF-188 | −1.48 | 5.29 | 33563 | 0.0367 |
| LRF-189 | −1.93 | 7.37 | 12207 | 0.0095 |
| LRF-190 | −1.36 | 5.53 | 49258 | 0.0117 |
| LRF-191 | −1.20 | 5.11 | 46325 | 0.0157 |
| LRF-192 | −5.85 | 5.95 | 50039 | 0.0073 |
| LRF-193 | −4.93 | 5.49 | 45283 | 0.0073 |
| LRF-194 | −7.08 | 7.31 | 33644 | 0.0073 |
| LRF-195 | −6.18 | 8.68 | 12312 | 0.0075 |
| LRF-196 | −4.10 | 7.28 | 50744 | 0.0075 |
| LRF-197 | −5.93 | 6.13 | 29157 | 0.0075 |
| LRF-198 | −6.60 | 5.89 | 43867 | 0.0075 |
| LRF-199 | −5.25 | 6.77 | 14743 | 0.0075 |
| LRF-200 | −5.05 | 7.00 | 34107 | 0.0075 |
| LRF-201 | −4.65 | 5.38 | 46989 | 0.0075 |
| LRF-202 | −3.83 | 6.25 | 57862 | 0.0075 |
| LRF-203 | −1.92 | 5.76 | 45950 | 0.0095 |
| LRF-204 | −4.72 | 6.00 | 42366 | 0.0097 |
| LRF-205 | −1.35 | 6.53 | 28420 | 0.0119 |
| LRF-206 | −1.96 | 6.06 | 26022 | 0.0119 |
| LRF-207 | −1.47 | 5.64 | 26912 | 0.0119 |
| LRF-208 | −1.45 | 5.55 | 49559 | 0.0122 |
| LRF-209 | −1.63 | 5.70 | 17546 | 0.0122 |
| LRF-210 | −1.80 | 5.68 | 111201 | 0.0119 |
| LRF-211 | −1.47 | 6.72 | 84306 | 0.0122 |
| LRF-212 | −1.65 | 6.70 | 109449 | 0.0216 |
| LRF-213 | −1.65 | 6.35 | 112327 | 0.0216 |
| LRF-214 | −1.28 | 5.97 | 62865 | 0.0212 |
| LRF-215 | −1.57 | 4.89 | 47633 | 0.0278 |
| LRF-216 | −3.41 | 5.31 | 34645 | 0.0449 |
| LRF-412 | −1.58 | 5.68 | 20733 | 0.0119 |
| LRF-413 | −2.02 | 7.62 | 29160 | 0.0119 |
| LRF-414 | −1.69 | 7.74 | 60576 | 0.0122 |
| LRF-415 | −1.47 | 7.53 | 60629 | 0.0122 |
| LRF-416 | −1.38 | 7.41 | 60589 | 0.0212 |
| LRF-417 | −1.87 | 7.47 | 104985 | 0.0212 |
| LRF-418 | −1.48 | 7.09 | 53919 | 0.0216 |
| LRF-419 | −1.35 | 6.16 | 53926 | 0.0153 |
| LRF-420 | −1.78 | 5.49 | 38574 | 0.0216 |
| LRF-421 | −1.23 | 6.44 | 33582 | 0.0356 |
| LRF-422 | −1.22 | 6.81 | 102828 | 0.0160 |
| LRF-423 | −1.57 | 5.74 | 23145 | 0.0122 |
| LRF-424 | −1.55 | 5.12 | 87641 | 0.0122 |
| LRF-425 | −1.54 | 6.15 | 101323 | 0.0119 |
| LRF-426 | −1.34 | 5.67 | 40610 | 0.0119 |
| LRF-427 | −2.26 | 6.12 | 38377 | 0.0122 |
| LRF-428 | −1.26 | 5.71 | 36256 | 0.0122 |
| LRF-429 | −1.28 | 6.43 | 32853 | 0.0216 |
| LRF-430 | −1.15 | 5.26 | 29492 | 0.0361 |
| LRF-431 | −3.59 | 5.20 | 15441 | 0.0122 |
| LRF-432 | −1.87 | 6.38 | 11973 | 0.0367 |

Table XLVII. LRFs Increased in Blood of Subjects Having Early Liver Response

TABLE XLVII

| | Relative Abundance | | | |
|---|---|---|---|---|
| | (foreground over | | | P value (Rank Sum |
| LRF# | background) | pI | MW (Da) | Test) |
| LRF-239 | 1.36 | 6.34 | 89862 | 0.0119 |
| LRF-240 | 1.36 | 6.40 | 32699 | 0.0367 |
| LRF-241 | 1.33 | 4.81 | 55544 | 0.0119 |
| LRF-242 | 1.76 | 8.75 | 11032 | 0.0122 |
| LRF-243 | 1.56 | 6.28 | 40990 | 0.0117 |
| LRF-244 | 1.38 | 4.66 | 17877 | 0.0119 |
| LRF-245 | 1.84 | 6.59 | 93897 | 0.0122 |
| LRF-246 | 1.66 | 6.14 | 56439 | 0.0109 |
| LRF-247 | 1.86 | 5.73 | 49301 | 0.0117 |
| LRF-248 | 1.56 | 5.28 | 33858 | 0.0361 |
| LRF-249 | 1.58 | 5.10 | 48641 | 0.0449 |
| LRF-433 | 7.28 | 6.10 | 33731 | 0.0075 |
| LRF-434 | 1.57 | 5.00 | 49123 | 0.0356 |
| LRF-435 | 1.47 | 5.92 | 139434 | 0.0367 |
| LRF-436 | 1.23 | 7.22 | 22990 | 0.0095 |
| LRF-437 | 1.64 | 5.22 | 39051 | 0.0122 |
| LRF-438 | 1.67 | 4.96 | 64411 | 0.0122 |
| LRF-439 | 1.28 | 7.51 | 10587 | 0.0361 |
| LRF-440 | 1.32 | 4.64 | 29999 | 0.0465 |
| LRF-441 | 14.60 | 5.22 | 35024 | 0.0075 |
| LRF-442 | 3.38 | 5.62 | 36992 | 0.0122 |

Table XLVIII. LRFs Decreased in Blood of Subjects Having Full Manifestation Liver Response

| | Relative Abundance | | | |
|---|---|---|---|---|
| | (foreground over | | | P value (Rank Sum |
| LRF# | background) | pI | MW (Da) | Test) |
| LRF-268 | −7.90 | 7.32 | 32623 | 0.0075 |
| LRF-269 | −3.85 | 4.99 | 21496 | 0.0075 |
| LRF-273 | −5.28 | 5.03 | 24476 | 0.0073 |
| LRF-274 | −4.83 | 6.10 | 29306 | 0.0073 |
| LRF-275 | −4.05 | 5.76 | 45171 | 0.0075 |
| LRF-276 | −7.83 | 7.00 | 32795 | 0.0075 |
| LRF-277 | −1.62 | 6.14 | 33891 | 0.0097 |
| LRF-278 | −3.10 | 7.72 | 46632 | 0.0112 |
| LRF-279 | −1.55 | 5.50 | 44208 | 0.0119 |
| LRF-280 | −1.37 | 4.70 | 50480 | 0.0119 |
| LRF-281 | −1.62 | 5.88 | 130261 | 0.0119 |
| LRF-282 | −1.44 | 5.47 | 68795 | 0.0122 |
| LRF-283 | −1.42 | 6.41 | 32810 | 0.0122 |
| LRF-284 | −2.04 | 6.03 | 32803 | 0.0122 |
| LRF-285 | −2.09 | 5.99 | 53879 | 0.0122 |
| LRF-286 | −2.08 | 4.96 | 115926 | 0.0178 |
| LRF-287 | −1.37 | 6.16 | 107251 | 0.0212 |
| LRF-288 | −3.32 | 5.05 | 28366 | 0.0073 |
| LRF-289 | −1.93 | 5.19 | 43558 | 0.0097 |
| LRF-290 | −1.60 | 7.13 | 101347 | 0.0097 |
| LRF-291 | −1.39 | 7.60 | 68219 | 0.0119 |
| LRF-292 | −1.60 | 6.97 | 102180 | 0.0119 |
| LRF-412 | −1.82 | 5.68 | 20733 | 0.0122 |
| LRF-413 | −1.76 | 7.62 | 29160 | 0.0122 |
| LRF-414 | −1.72 | 7.74 | 60576 | 0.0122 |
| LRF-415 | −1.41 | 7.53 | 60629 | 0.0122 |
| LRF-416 | −1.29 | 7.41 | 60589 | 0.0122 |
| LRF-417 | −1.59 | 7.47 | 104985 | 0.0465 |
| LRF-418 | −1.86 | 7.09 | 53919 | 0.0122 |
| LRF-419 | −1.74 | 6.16 | 53926 | 0.0117 |
| LRF-420 | −2.21 | 5.49 | 38574 | 0.0122 |
| LRF-421 | −1.39 | 6.44 | 33582 | 0.0196 |
| LRF-422 | −1.41 | 6.81 | 102828 | 0.0119 |
| LRF-423 | −1.84 | 5.74 | 23145 | 0.0122 |
| LRF-424 | −2.73 | 5.12 | 87641 | 0.0119 |
| LRF-425 | −1.77 | 6.15 | 101323 | 0.0114 |
| LRF-426 | −2.08 | 5.67 | 40610 | 0.0122 |
| LRF-427 | −2.80 | 6.12 | 38377 | 0.0122 |
| LRF-428 | −1.62 | 5.71 | 36256 | 0.0122 |
| LRF-429 | −1.32 | 6.43 | 32853 | 0.0216 |
| LRF-430 | −1.36 | 5.26 | 29492 | 0.0117 |
| LRF-431 | −9.14 | 5.20 | 15441 | 0.0073 |
| LRF-432 | −2.16 | 6.38 | 11973 | 0.0119 |
| LRF-435 | 1.47 | 5.92 | 139434 | 0.0367 |
| LRF-443 | −1.42 | 5.05 | 51630 | 0.0119 |
| LRF-444 | −1.17 | 5.49 | 27626 | 0.0160 |
| LRF-445 | −1.54 | 6.95 | 77438 | 0.0216 |
| LRF-446 | −1.44 | 5.26 | 27300 | 0.0361 |
| LRF-447 | −3.56 | 7.01 | 21885 | 0.0097 |
| LRF-448 | −1.86 | 5.69 | 21268 | 0.0119 |
| LRF-449 | −1.72 | 5.49 | 40308 | 0.0119 |
| LRF-450 | −1.82 | 6.38 | 91246 | 0.0119 |
| LRF-451 | −1.67 | 6.42 | 21447 | 0.0122 |
| LRF-452 | −1.37 | 5.50 | 36402 | 0.0122 |
| LRF-453 | −1.52 | 7.33 | 58295 | 0.0122 |
| LRF-454 | −2.26 | 7.10 | 58561 | 0.0122 |
| LRF-455 | −1.87 | 7.55 | 63296 | 0.0122 |
| LRF-456 | −2.11 | 7.22 | 106487 | 0.0122 |
| LRF-457 | −1.66 | 6.79 | 38847 | 0.0117 |
| LRF-465 | −1.92 | 7.00 | 72422 | 0.0119 |
| LRF-466 | −1.80 | 6.93 | 22909 | 0.0119 |
| LRF-467 | −4.08 | 7.59 | 78393 | 0.0075 |
| LRF-468 | −1.44 | 5.33 | 107726 | 0.0122 |

Table XLIX. LRFs Increased in Blood of Subjects Having Full Manifestation Liver Response

| | Relative Abundance | | | |
|---|---|---|---|---|
| | (foreground over | | | P value (Rank Sum |
| LRF# | background) | pI | MW (Da) | Test) |
| LRF-302 | 6.23 | 5.03 | 17949 | 0.0073 |
| LRF-308 | 4.95 | 5.81 | 58847 | 0.0075 |
| LRF-309 | 1.36 | 6.05 | 26359 | 0.0097 |
| LRF-310 | 2.57 | 7.74 | 94407 | 0.0097 |
| LRF-311 | 5.09 | 6.64 | 50367 | 0.0073 |
| LRF-312 | 5.09 | 6.94 | 38244 | 0.0073 |
| LRF-313 | 4.63 | 6.42 | 31755 | 0.0073 |
| LRF-314 | 3.15 | 4.80 | 33553 | 0.0119 |
| LRF-315 | 3.19 | 4.31 | 11695 | 0.0122 |
| LRF-316 | 1.18 | 5.02 | 25265 | 0.0178 |
| LRF-317 | 1.28 | 7.43 | 49167 | 0.0196 |
| LRF-318 | 1.98 | 4.92 | 27246 | 0.0216 |
| LRF-319 | 4.52 | 5.64 | 43678 | 0.0073 |
| LRF-320 | 5.92 | 6.36 | 46476 | 0.0075 |
| LRF-321 | 1.33 | 6.74 | 78780 | 0.0119 |
| LRF-433 | 2.06 | 6.10 | 33731 | 0.0097 |
| LRF-434 | 1.79 | 5.00 | 49123 | 0.0122 |
| LRF-436 | 1.39 | 7.22 | 22990 | 0.0119 |
| LRF-437 | 1.33 | 5.22 | 39051 | 0.0361 |
| LRF-438 | 1.36 | 4.96 | 64411 | 0.0216 |
| LRF-439 | 1.28 | 7.51 | 10587 | 0.0361 |
| LRF-440 | 1.32 | 4.64 | 29999 | 0.0465 |
| LRF-441 | 14.60 | 5.22 | 35024 | 0.0075 |
| LRF-442 | 3.38 | 5.62 | 36992 | 0.0122 |
| LRF-458 | 1.80 | 5.02 | 68091 | 0.0367 |
| LRF-459 | 4.34 | 7.27 | 33439 | 0.0254 |
| LRF-460 | 1.67 | 8.10 | 11240 | 0.0122 |
| LRF-461 | 1.63 | 4.95 | 32578 | 0.0465 |
| LRF-462 | 1.65 | 4.63 | 21342 | 0.0122 |
| LRF-463 | 4.06 | 4.94 | 32968 | 0.0122 |
| LRF-464 | 2.56 | 4.85 | 69401 | 0.0122 |
| LRF-469 | 1.28 | 7.00 | 64791 | 0.0112 |
| LRF-470 | 1.47 | 7.72 | 50141 | 0.0119 |
| LRF-471 | 1.58 | 7.45 | 31833 | 0.0119 |
| LRF-472 | 6.66 | 5.75 | 40211 | 0.0073 |
| LRF-473 | 4.54 | 6.44 | 25556 | 0.0073 |
| LRF-474 | 6.71 | 7.68 | 23581 | 0.0073 |

Table L. LRFs Decreased in Blood of Subjects Having Late Liver Response

| | Relative Abundance | | | |
|---|---|---|---|---|
| | (foreground over | | | P value (Rank Sum |
| LRF# | background) | pI | MW (Da) | Test) |
| LRF-337 | −4.41 | 5.19 | 46770 | 0.0122 |
| LRF-340 | −4.43 | 6.63 | 142885 | 0.0073 |
| LRF-341 | −3.78 | 6.17 | 31990 | 0.0075 |
| LRF-342 | −2.36 | 6.79 | 34138 | 0.0095 |
| LRF-343 | −1.49 | 5.64 | 26847 | 0.0117 |
| LRF-344 | −1.76 | 6.44 | 113112 | 0.0119 |
| LRF-345 | −1.52 | 5.98 | 107135 | 0.0119 |
| LRF-346 | −1.64 | 6.17 | 106618 | 0.0119 |
| LRF-347 | −1.54 | 6.31 | 105250 | 0.0119 |
| LRF-348 | −2.19 | 5.32 | 12352 | 0.0122 |
| LRF-349 | −2.00 | 6.63 | 112785 | 0.0122 |
| LRF-350 | −1.63 | 6.07 | 106848 | 0.0122 |
| LRF-351 | −1.51 | 6.27 | 33192 | 0.0122 |
| LRF-352 | −1.23 | 7.57 | 47111 | 0.0153 |
| LRF-353 | −3.34 | 5.88 | 49545 | 0.0075 |
| LRF-354 | −1.97 | 5.90 | 23350 | 0.0122 |
| LRF-418 | −1.73 | 7.09 | 53919 | 0.0373 |
| LRF-420 | −1.54 | 5.49 | 38574 | 0.0200 |
| LRF-425 | −1.51 | 6.15 | 101323 | 0.0122 |
| LRF-426 | −1.95 | 5.67 | 40610 | 0.0122 |
| LRF-427 | −1.33 | 6.12 | 38377 | 0.0122 |
| LRF-428 | −1.60 | 5.71 | 36256 | 0.0119 |
| LRF-429 | −1.44 | 6.43 | 32853 | 0.0122 |
| LRF-430 | −1.22 | 5.26 | 29492 | 0.0361 |
| LRF-431 | −1.83 | 5.20 | 15441 | 0.0122 |
| LRF-432 | −1.37 | 6.38 | 11973 | 0.0465 |
| LRF-435 | −2.68 | 5.92 | 139434 | 0.0122 |
| LRF-443 | −1.29 | 5.05 | 51630 | 0.0491 |
| LRF-444 | −1.19 | 5.49 | 27626 | 0.0358 |
| LRF-445 | −1.37 | 6.95 | 77438 | 0.0195 |
| LRF-446 | −1.16 | 5.26 | 27300 | 0.0373 |
| LRF-447 | −2.99 | 7.01 | 21885 | 0.0097 |
| LRF-448 | −1.53 | 5.69 | 21268 | 0.0119 |
| LRF-449 | −1.60 | 5.49 | 40308 | 0.0122 |
| LRF-450 | −1.44 | 6.38 | 91246 | 0.0212 |
| LRF-451 | −1.74 | 6.42 | 21447 | 0.0122 |
| LRF-452 | −1.40 | 5.50 | 36402 | 0.0119 |
| LRF-453 | −1.91 | 7.33 | 58295 | 0.0216 |
| LRF-454 | −1.87 | 7.10 | 58561 | 0.0278 |
| LRF-455 | −1.88 | 7.55 | 63296 | 0.0122 |
| LRF-456 | −1.68 | 7.22 | 106487 | 0.0367 |
| LRF-457 | −1.54 | 6.79 | 38847 | 0.0216 |

Table LI. LRFs Increased in Blood of Subjects Having Late Liver Response

| | Relative Abundance | | | |
|---|---|---|---|---|
| | (foreground over | | | P value (Rank Sum |
| LRF# | background) | pI | MW (Da) | Test) |
| LRF-374 | 2.23 | 5.73 | 62948 | 0.0097 |
| LRF-375 | 4.03 | 6.10 | 36098 | 0.0104 |
| LRF-376 | 4.13 | 5.57 | 34369 | 0.0104 |
| LRF-377 | 4.80 | 4.93 | 47664 | 0.0073 |
| LRF-378 | 4.60 | 5.33 | 34378 | 0.0075 |
| LRF-379 | 6.54 | 5.79 | 15434 | 0.0075 |
| LRF-380 | 9.34 | 4.63 | 55582 | 0.0075 |
| LRF-381 | 2.80 | 5.25 | 25040 | 0.0095 |
| LRF-382 | 2.12 | 5.51 | 34262 | 0.0112 |
| LRF-383 | 1.55 | 7.70 | 46543 | 0.0122 |
| LRF-384 | 2.81 | 5.06 | 12383 | 0.0192 |
| LRF-385 | 1.42 | 4.85 | 14311 | 0.0160 |
| LRF-419 | 1.32 | 6.16 | 53926 | 0.0189 |
| LRF-458 | 1.85 | 5.02 | 68091 | 0.0119 |
| LRF-459 | 6.91 | 7.27 | 33439 | 0.0108 |
| LRF-460 | 1.75 | 8.10 | 11240 | 0.0122 |
| LRF-461 | 1.62 | 4.95 | 32578 | 0.0216 |
| LRF-462 | 2.10 | 4.63 | 21342 | 0.0122 |
| LRF-463 | 1.57 | 4.94 | 32968 | 0.0122 |
| LRF-464 | 1.80 | 4.85 | 69401 | 0.0367 |
| LRF-476 | 3.84 | 6.24 | 41308 | 0.0108 |

We claim:

1. A method for: (i) screening or diagnosing a liver response in a subject, (ii) determining the stage or severity of a liver response in a subject, (iii) identifying a subject at risk of developing liver response, or (iv) monitoring the effect of therapy administered to a subject having liver response, said method comprising detecting within a test biological sample from the subject a Liver Response-Associated Protein Isoforms (LRPIs) selected from the group consisting of: LRPI-1.1, LRPI-2.1, LRPI-3.1, LRPI-4.1, LRPI-6.1, LRPI-8.1, LRPI-9.1, LRPI-10.1, LRPI-11.1, LRPI-11.2, LRPI-11.3, LRPI-12.1, LRPI-13.1, LRPI-14.1, LRPI-15.1, LRPI-16.1, LRPI-17.1, LRPI-17.2, LRPI-1 8.1, LRPI-19.1, LRPI-20.1, LRPI-21.1, LRPI-22.1, LRPI-23.1, LRPI-24.1, LRPI-25.1, LRPI-26.1, LRPI-27.1, LRPI-28.1, LRPI-29.1, LRPI-30.1, LRPI-31.1, LRPI-32.1, LRPI-36.1, LRPI-37.1, LRPI-38.1, LRPI-53.1, LRPI-54.1, LRPI-56.1, LRPI-56.2, LRPI-60.1, LRPI-61.1, LRPI-63.1, LRPI-64.1, LRPI-66.1, LRPI-67.1, LRPI-69.1, LRPI-70.1, LRPI-71.1, LRPI-72.1, LRPI-73.1, LRPI-74.1, LRPI-75.1, LRPI-75.2, LRPI-76.1, LRPI-77.1, LRPI-78.1, LRPI-79.1, LRPI-79.2, LRPI-80.1, LRPI-81.1, LRPI-83.1, LRPI-84.1, LRPI-85.1, LRPI-87.1, LRPI-88.1, LRPI-89.1,LRPI-90.1,LRPI-91.1,LRPI-92.1,LRPI-107.1, LRPI-109.1,LRPI-110.1,LRPI-111.1, LRPI-112.1, LRPI-112.2, LRPI-113.1, LRPI-114.1, LRPI-115.1, LRPI-115.2, LRPI-119.1, LRPI-119.2, LRPI-119.3, LRPI-120.1, LRPI-121.1, LRPI-122.1, LRPI-123.1, LRPI-123.2, LRPI-124.1, LRPI-126.1, LRPI-127.1, LRPI-128.1, LRPI-129.1, LRPI-130.1, LRPI-131.1, LRPI-132.1, LRPI-132.2, LRPI-133.1, LRPI-134.1, LRPI-156.1, LRPI-157.1, LRPI-158.1, LRPI-159.1, LRPI-160.1, LRPI-161.1, LRPI-162.1, LRPI-163.1, LRPI-164.1, LRPI-165.1, LRPI-166.1, LRPI-167.1, LRPI-168.1, LRPI-169.1, LRPI-170.1, LRPI-172.1, LRPI-173.1, LRPI-174.1, LRPI-175.1, LRPI-175.2, LRPI-176.1, LRPI-177.1, LRPI-178.1, LRPI-179.1, LRPI-181.1, LRPI-182.1, LRPI-183.1, LRPI-184.1, LRPI-184.2, LRPI-217.1, LRPI-217.3, LRPI-218.1, LRPI-219.1, LRPI-220.1, LRPI-221.1, LRPI-222.1, LRPI-223.1, LRPI-224.1, LRPI-225.1, LRPI-226.1, LRPI-227.1, LRPI-228.1, LRPI-230.1, LRPI-231.1, LRPI-232.1, LRPI-232.2, LRPI-234.1, LRPI-235.1, LRPI-236.1, LRPI-237.1, LRPI-256.1, LRPI-257.1, LRPI-258.1, LRPI-259.1, LRPI-260.1, LRPI-261.1, LRPI-262.1, LRPI-263.1, LRPI-264.1, LRPI-265.1, LRPI-266.1, LRPI-270.1, LRPI-271.1, LRPI-294.1, LRPI-295.1, LRPI-296.1, LRPI-297.1, LRPI-298.1, LRPI-300.1, LRPI-300.2, LRPI-301.1, LRPI-303.1, LRPI-305.1, LRPI-306.1, LRPI-328.1, LRPI-329.1, LRPI-331.1, LRPI-332.1, LRPI-334.1, LRPI-335.1, LRPI-356.1, LRPI-358.1, LRPI-359.1, LRPI-360.1, LRPI-361.1, LRPI-363.1, LRPI-364.1, LRPI-365.1, LRPI-366.1, LRPI-367.1, LRPI-368.1, LRPI-369.1, LRPI-370.1, LRPI-371.1, LRPI-372.1, LRPI-373.1, LRPI-386.1, LRPI-387.1, LRPI-400.1, LRPI-400.2, LRPI-389.1, LRPI-390.1, LRPI-391.1, LRPI-392.1, LRPI-393.1, LRPI-394.1, LRPI-394.2, LRPI-395.1, LRPI-395.2, LRPI-397.1, LRPI-399.1, LRPI-401.1, LRPI-402.1, LRPI-403.1, LRPI-405.1, LRPI-405.2, LRPI-407.1, LRPI-409.1, LRPI-410.1, which has an activity or level indicative of a liver response.

2. The method according to claim 1, wherein the biological sample is blood, serum, plasma or liver tissue.

3. A method for determining whether an agent induces a liver response comprising:
a) exposing a target cell, tissue or biological organism to the agent;
b) obtaining a test biological sample comprised of the target cell, tissue or biological organism;
c) detecting and/or quantifying the level or activity of an LRPI in the sample; and
d) comparing the level or activity of the LRPI in the test sample with the level or activity of said LRPI in a control sample, or with a previously determined reference range for said LRPI in targets free from liver response, or with the level or activity of at least one ERF in the test sample.

4. The method according to claim 3 wherein the control sample is obtained from a target which has not been exposed to the agent.

5. The method according to claim 3 wherein the agent is a drug.

6. The method according to claim 5 wherein step a) comprises administering a drug to the biological organism.

7. The method according to claim 5 wherein the target is a mammal.

8. The method of claim 7 wherein the mammal is a rodent.

9. The method of claim 8 wherein the rodent is a rat.

10. The method according to claim 5 wherein the test sample is tissue, blood, serum, plasma or urine.

11. The method according to claim 5, wherein the step of quantitatively detecting comprises testing at least one aliquot of the test biological sample, said step of testing comprising:
(a) contacting the aliquot with an antibody that is immunospecific for a LRPI;
(b) quantitatively measuring the binding of the antibody and the LRPI; and
(c) comparing the results of step (b) with a predetermined reference range.

12. The method according to claim 11, wherein the antibody is a monoclonal, chimeric, bispecific or humanised antibody.

13. The method according to claim 12, wherein the step of quantitatively detecting comprises testing a plurality of aliquots with a plurality of antibodies cognate for a plurality of preselected LRPIs.

14. The method according to any one of claims 11 wherein the antibody is immobilized on a solid or semi-solid support.

15. The method according to claim 11 wherein the antibody is provided within a spatial array of antibodies.

16. A kit comprising one or more antibodies capable of immunospecific binding to a Liver Response-Associated Protein Isoform (LRPI)s, other reagents and instructions for use.

17. A method for screening or diagnosing of liver response in a subject or for monitoring the effect of a drug or therapy administered to a subject, comprising:
(a) contacting at least one oligonucleotide probe comprising 10 or more consecutive nucleotides complementary to a nucleotide sequence encoding an LRPI with RNA obtained from a biological sample from the subject or with cDNA copied from the RNA wherein said contacting occurs under conditions that permit hybridization of the probe to the nucleotide sequence if present;
(b) detecting hybridization, if any, between the probe and the nucleotide sequence; and
(c) comparing the hybridization, if any, detected in step (b) with the hybridization detected in a control sample, or with a previously determined reference range.

18. A method of claim 17, wherein step (a) includes the step of hybridizing the nucleotide sequence to a DNA array, wherein one or more members of the array are the probes complementary to a plurality of nucleotide sequences encoding distinct LRPIs.
